SEQUENCE LISTING (10 sequences)

SEQ ID NO: 1 - protein, 353 aa, Escherichia coli
MAIDENKQKALAAALGQIEKQFGKGSIMRLGEDRSMDVETISTGSLSLDIALGAGGLPMGRIVEIYGPESSGKTTLTLQVIAAAQREGKTCAFIDAEHALDPIYARKLGVDIDNLLCSQPDTGEQALEICDALARSGAVDVIVVDSVAALTPKAEIEGEIGDSHMGLAARMMSQAMRKLAGNLKQSNTLLIFINQIRMKIGVMFGNPETTTGGNALKFYASVRLDIRRIGAVKEGENVVGSETRVKVVKNKIAAPFKQAEFQILYGEGINFYGELVDLGVKEKLIEKAGAWYSYKGEKIGQGKANATAWLKDNPETAKEIEKKVRELLLSNPNSTPDFSVDDSEGVAETNEDF

SEQ ID NO: 2 - protein, 31 aa, Escherichia coli
AIDENKQKALAAALGQIEKQFGKGSIMRLGE

SEQ ID NO: 3 - protein, 16 aa, Artificial Sequence
  Polypeptide mimetic of bacterial RecA N-terminal helical domain.
XXXXXXXXXXXXXXXX

SEQ ID NO: 4 - protein, 31 aa, Artificial Sequence
  Avian pancreatic polypeptide (aPP) folding residues grafted with bacterial RecA N-termiuns self-interaction contact residues.
GPSQPTYPGDDAPKEDLLRFALNLIQYLFVV

SEQ ID NO: 5 - protein, 31 aa, Artificial Sequence
  Artificialy constructed polypeptide homologous to alpha-helix of avian pancreatic polypeptide (aPP).
GPSQPTYPGDDAPVEDLIRFYNDLQQYLNVV

SEQ ID NO: 6 - protein, 30 aa, Escherichia coli
AIDENKQKALAAALGQIEKQFGKGSIMRLG

SEQ ID NO: 7 - protein, 7 aa, Artificial Sequence
  Artificially constructed polypeptide comprising an endoplasmic reticulum retention motif.
CFFKDEL

SEQ ID NO: 8 - protein, 26 aa, Artificial Sequence
  Synthesized peptide mimetic of bacterial RecA N-terminus helix.
DKQKALAKALEKIAKQFGKVTVMRTT

SEQ ID NO: 9 - protein, 26 aa, Artificial Sequence
  Synthesized peptide mimetic of bacterial RecA N-terminus helix.
DKQKALAKALEKIAKQFGKVTVCRTT

SEQ ID NO: 10 - protein, 27 aa, Artificial Sequence
  Synthesized peptide mimetic of bacterial RecA N-terminus helix.
DKQKALAKALEKICKQFCGKVTVCRTT

(19) United States
(12) Patent Application Publication
(10) Pub. No.: US 2006/0199768 A1
(43) Pub. Date: Sep. 7, 2006

(54) Inhibitors of RecA activities for control of antibiotic-resistant bacterial pathogens

(75) Inventor: Scott Fain Singleton, Chapel Hill, NC (US)

Correspondence Address: JENKINS, WILSON, TAYLOR & HUNT, P. A., 3100 TOWER BLVD, SUITE 1200, DURHAM, NC, 27707, US

(73) Assignee: University of North Carolina at Chapel Hill

(21) Appl. No.: 11/371,373

(22) Filed: Mar. 7, 2006

Related U.S. Application Data
(60) Provisional application No. 60/659,340, filed on Mar. 7, 2005.
(60) Provisional application No. 60/660,847, filed on Mar. 11, 2005.

(51) Int. Cl. A61K 38/10; A61K 31/7076; A61K 31/7072; A61K 31/675; A61K 31/12; C07K 7/08; C07F 9/6512; C07H 19/16

(52) U.S. Cl. 514/15; 514/45; 530/326; 536/27.3; 514/81; 514/85; 544/243; 544/244; 514/679

(57) ABSTRACT

Compounds for modulating RecA protein activity are provided. In some embodiments, the compounds modulate RecA activity by interfering with assembly of monomeric RecA protein subunits into a nucleoprotein filament. In some embodiments, the compounds modulate RecA activity by interfering with adenosine triphosphate hydrolysis by the RecA protein. Methods of screening for and methods of using the compounds are also provided.

### CROSS REFERENCE TO RELATED APPLICATIONS

This application is based on and claims priority to U.S. Provisional Patent Application Ser. No. 60/659,340, filed Mar. 7, 2005, which is incorporated herein by reference in its entirety and to U.S. Provisional Patent Application Ser. No. 60/660,847, filed Mar. 11, 2005, which is incorporated herein by reference in its entirety.

### TECHNICAL FIELD

The currently disclosed subject matter relates to methods and compounds for modulating RecA protein activity. In some embodiments, the presently disclosed methods and compositions provide for modulation of RecA activity by interfering with assembly of monomeric RecA protein subunits into a nucleoprotein filament and/or by interfering with adenosine triphosphate hydrolysis by the RecA protein.

### ABBREVIATIONS

° C.=degrees Celsius
μL=microliter
μM =micromolar
ADP=adenosine diphosphate
aPP=avian pancreatic polypeptide
ATP=adenosine triphosphate
ATP=adenosine triphosphate
BAR=boronic acid resin
BCNU=Bischloroethyinitrosourea
cm=centimeter
Gc=Gonococci
GFP=green fluorescent protein
HGT=horizontal DNA transfer
HTS=high throughput screen
ICE=integrative and conjugative elements
IRA=inhibitor of RecA activity
L-DTT=1,4-dithio-L-threitol
MB-CHO=o-methoxybenzaldehyde
MHz=megahertz
MIC=minimum inhibitory concentration
mm=millimeter
MMC=mytomycin C
mmol=micromole
NADH=Nicotinamide adenine dinucleotide
NMR=nuclear magnetic resonance
NPF=nucleoprotein filament
OD=optical density
PI=propidium iodide
Pmol=picomole
PPM=parts per million
RDR=recombinational DNA repair
RT=room temperature
SA-PMP=streptavidin-paramagnetic particles
SPOS=parallel solid-phase synthesis
ssDNA=single-stranded deoxyribonucleic acid
STD=sexually transmitted disease
WT=wild-type

### BACKGROUND

Since the introduction of penicillin to clinical medicine 60 years ago, pharmaceutical companies have produced more than 100 antibacterial agents/antibiotics to combat a wide variety of bacterial infections. By the 1980s, it was believed that industrialized nations had won the war against pathogenic microbes. However, in the past several years, the rapid emergence of bacterial resistance to antibiotics has been observed.

The extensive use (and misuse) of antibiotics has provided powerful forces for the selection of microbes that either carried mutations conferring resistance or had the enhanced ability to mutate to resistance in the face of the antibiotic. Bacteria have mutated or have acquired new genes producing novel machinery to overcome the action of many antibiotics. Indeed, bacteria that are resistant to newly introduced drugs have consistently appeared a few years after the introduction of each new drug to clinical use.

Antibiotic resistance has enormous human and economic consequences worldwide, and is estimated to cost between $5 billion and $24 billion each year in the United States alone. In 1992, 19,000 deaths in the United States were attributed to antibiotic resistance, making it the eleventh leading cause of death nationwide. Alarmingly, the rate of resistance is accelerating dramatically.

The rapid rate at which bacteria develop drug resistance is due in large part to mutations arising during stress-induced DNA repair, as well as the lateral transfer of genes between organisms. The bacterial RecA protein is essential to both of these processes and its functions are unique to bacteria. In addition to suppressing DNA repair, inhibiting RecA activity in the cell could abrogate horizontal gene transfer, SOS mutagenesis, and stationary phase mutation. In addition, RecA function is required for aspects of pathogenicity, including antibiotic-induced responses to ciprofloxacin and β-lactams, antigenic variation in Neisseriae, and the induction of shiga toxin production. All RecA functions appear to require formation of an active nucleoprotein filament (NPF) comprising multiple RecA monomers, ATP, and single-stranded DNA (ssDNA).

Thus, an antibiotic strategy that targets RecA activity could represent an approach that bypasses the evolution of antibiotic resistance. The presently disclosed subject matter addresses this and other unmet needs in the art.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In one embodiment of the presently disclosed subject matter, a compound for modulating RecA protein activity is provided, wherein the compound modulates RecA activity by interfering with assembly of monomeric RecA protein subunits into a nucleoprotein filament. In some embodiments, the compound inhibits RecA activity. In some embodiments, the compound is a mimetic of the N-terminal helical domain of the RecA protein. In particular embodiments, the N-terminal helical domain of the RecA protein comprises amino acid residues 1-31 of the E. coli RecA protein. Further, in some embodiments, the compound comprises the amino acid sequence B—X₃-Z-X₂-Z-Z-X₂-Z-X₃-Z, wherein B is lysine or arginine; Xₙ is n number of any amino acids and X can be the same or different amino acids; and Z is alanine, valine, leucine, isoleucine, phenylalanine, or methionine. In some embodiments, the compound is selected as a mimetic of the N-terminal helical domain of the RecA protein utilizing an mRNA display assay.

In another embodiment of the presently disclosed subject matter, a compound for modulating RecA protein activity is provided, wherein the compound modulates RecA activity by interfering with adenosine triphosphate hydrolysis by the RecA protein. In some embodiments, the compound inhibits RecA activity. In some embodiments, the compound is selected from the group consisting of a modified adenosine, a modified 5-propynyl-deoxyuridine, a curcumin derivative and a bismuth-dithiol complex. In some embodiments, the compound is a modified adenosine selected from the group consisting of a modified adenosine monophosphate, a modified adenosine diphosphate and a modified adenosine triphosphate. In some embodiments, the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate.

In some embodiments, the compound has the general formula (I): 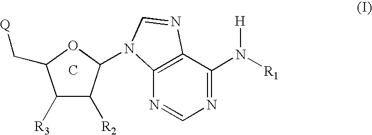
wherein:

R₁ is selected from the group consisting of: 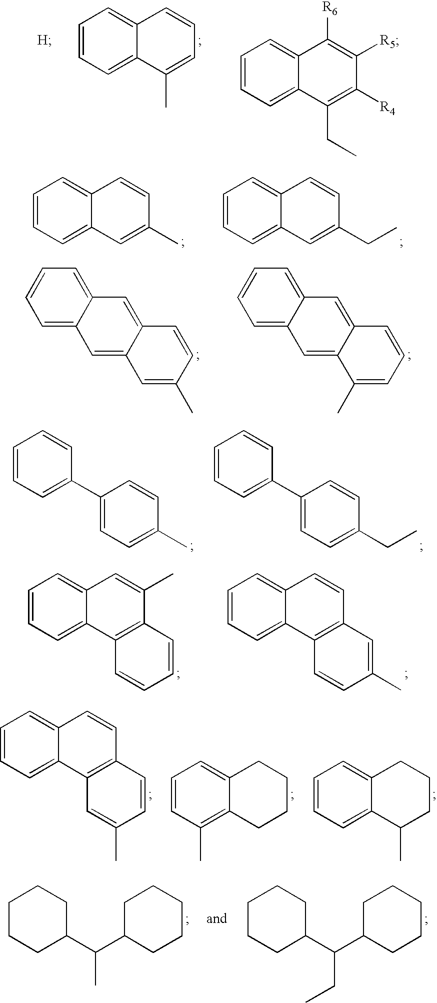
wherein R₄, R₅ and R₆ are each independently selected from the group consisting of H, alkyl, substituted alkyl, OH, alkoxyl, and substituted alkoxyl;

R₂ and R₃ are each independently selected from the group consisting of H, F, OH, NH₂ and Y-Z-R₇,

Y is selected from the group consisting of O and NR₂₂, and wherein R₂₂ is selected from the group consisting of H, alkyl, substituted alkyl, and alkoxyl;

Z is selected from the group consisting of (CH₂)ₚ, CF₂ and C═O, and wherein p is an integer from 1 to 8; and

R₇ is selected from the group consisting of H, alkyl, substituted alkyl, alkenyl, substituted alkenyl, alkynyl, substituted alkynyl, aryl and substituted aryl, or R₂ and R₃ can together with ring C form the following five-membered heterocyclic ring structure: 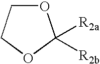
wherein R₂ₐ and R_{2b} are each independently selected from the group consisting of H, alkyl, substituted alkyl, alkenyl, substituted alkenyl, alkynyl, substituted alkynyl, aryl and substituted aryl; and

Q is selected from the group consisting of: 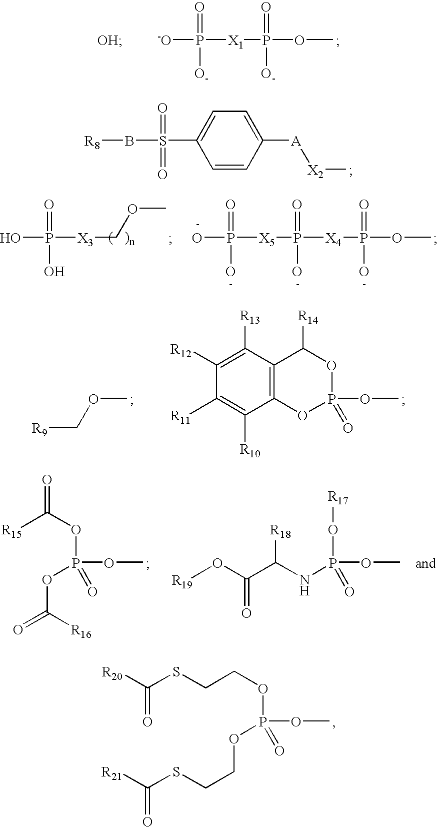
wherein X₁, X₂, X₃, X₄ and X₅ are each independently selected from the group consisting of O, NR₂₃, CH₂ and CF₂, and wherein R₂₃ is selected from the group consisting of H, alkyl, substituted alkyl, and alkoxyl;

A and B are each independently selected from the group consisting of O, NR₂₄, CH₂, CF₂ and C═O, and wherein R₂₄ is selected from the group consisting of H, alkyl, substituted alkyl, and alkoxyl;

n is an integer from 0 to 4;

R₈ is selected from the group consisting of H, alkyl, substituted alkyl, alkenyl, substituted alkenyl, alkynyl, substituted alkynyl, aryl and substituted aryl;

R₉ is selected from the group consisting of 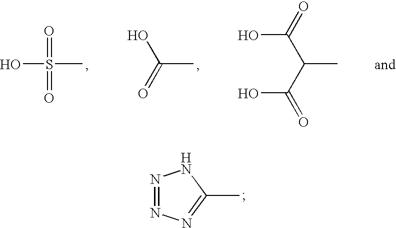
and

R₁₀, R₁₁, R₁₂, R₁₃, R₁₄, R₁₅, R₁₆, R₁₇, R₁₈, R₁₉, R₂₀ and R₂₁, are each independently selected from the group consisting of H, alkyl, substituted alkyl, alkenyl, substituted alkenyl, alkynyl, substituted alkynyl, aryl and substituted aryl; or a pharmaceutically acceptable salt thereof; subject to the proviso that the compound of formula (I) is not 2′,3′-O—(N-methyl-anthraniloyl)-adenosine-5′diphosphate, 2′,(3′)-O-(2,4,6-trinitrophenyl)-adenosine-5′-diphosphate, N⁶-(1-napthyl)-adenosine-5′-O-Diphosphate, N⁶-(1-benzyl)-adenosine-5′-O-Diphosphate, or N⁶-(2-phenethyl)-adenosine-5′-O-Diphosphate.

In some embodiments, the compound has the general formula (II): 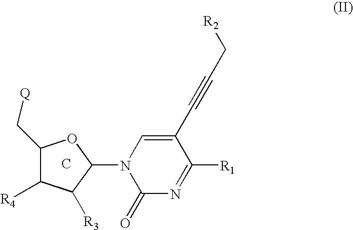
wherein:

R₁ and R₂ are each independently selected from the group consisting of H, alkyl, substituted alkyl, F, Cl, Br, OH, NR₂₂R₂₃ and Y-Z-R₂₄, wherein

R₂₂ and R₂₃ are each independently selected from the group consisting of H, alkyl, substituted alkyl, and alkoxyl;

Y is selected from the group consisting of O, S, NR₂₅ and (CH₂)ₚ, and wherein R₂₅ is selected from the group consisting of H, alkyl, substituted alkyl, and alkoxyl and p is an integer from 1 to 8;

Z can be present or absent and when present is selected from the group consisting of O, S, NR₂₆R₂₇ and C═O, and wherein R₂₆ and R₂₇ are each independently selected from the group consisting of H, alkyl, substituted alkyl, and alkoxyl; and

R₂₄ is selected from the group consisting of H, alkyl, substituted alkyl, alkenyl, substituted alkenyl, alkynyl, substituted alkynyl, aryl and substituted aryl;

R₃ and R₄ are each independently selected from the group consisting of H, F, OH, NR₂₈R₂₉ and R₅—R₆—R₇,

wherein R₂₈ and R₂₉ are each independently selected from the group consisting of H, alkyl, substituted alkyl, and alkoxyl;

R₅ is selected from the group consisting of O and NR₃₀, and wherein R₃₀ is selected from the group consisting of H, alkyl, substituted alkyl, and alkoxyl;

R₆ is selected from the group consisting of (CH2)_{q}, CF₂ and C═O, and wherein q is an integer from 1 to 8; and

R₇ can be present or absent and when present is selected from the group consisting of H, alkyl, substituted alkyl, alkenyl, substituted alkenyl, alkynyl, substituted alkynyl, aryl and substituted aryl, or

R₃ and R₄ can together with ring C form the following five-membered heterocyclic ring structure: 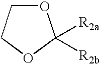
wherein R₂ₐ and R_{2b} are each independently selected from the group consisting of H, alkyl, substituted alkyl, alkenyl, substituted alkenyl, alkynyl, substituted alkynyl, aryl and substituted aryl; and

Q is selected from the group consisting of: 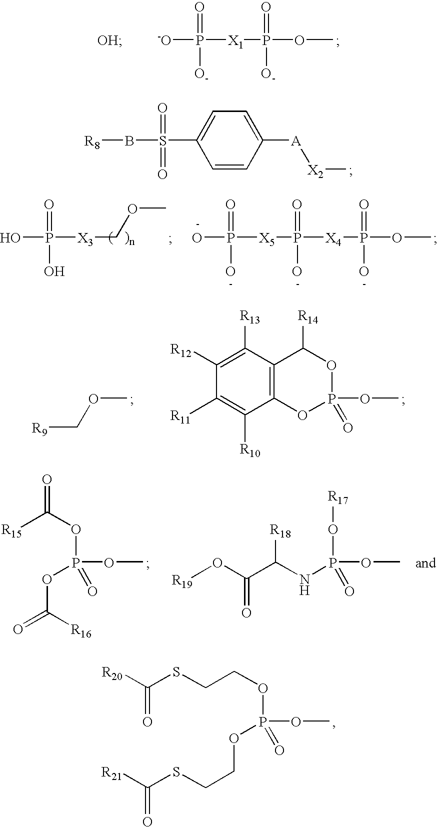
wherein X₁, X₂, X₃, X₄ and X₅ are each independently selected from the group consisting of O, NR₃₁, CH₂ and CF₂, and wherein R₃₁ is selected from the group consisting of H, alkyl, substituted alkyl, and alkoxyl;

A and B are each independently selected from the group consisting of O, NR₃₂, CH₂, CF₂ and C═O, and wherein R₃₂ is selected from the group consisting of H, alkyl, substituted alkyl, and alkoxyl;

n is an integer from 0 to 4;

R₈ is selected from the group consisting of H, alkyl, substituted alkyl, alkenyl, substituted alkenyl, alkynyl, substituted alkynyl, aryl and substituted aryl;

R₉ is selected from the group consisting of 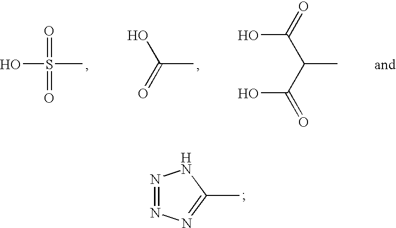
and

R₁₀, R₁₁, R₁₂, R₁₃, R₁₄, R₁₅, R₁₆, R₁₇, R₁₈, R₁₉, R₂₀ and R₂₁ are each independently selected from the group consisting of H, alkyl, substituted alkyl, alkenyl, substituted alkenyl, alkynyl, substituted alkynyl, aryl and substituted aryl; or a pharmaceutically acceptable salt thereof.

In some embodiments, the compound has the general formula (III): 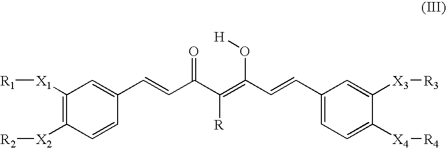
wherein:

X₁, X₂, X₃ and X₄ are each independently selected from the group consisting of H, F, (CH₂)ₙ, CF₂, C═O, O and NR₅, and wherein n is an integer from 1 to 8 and R₅is selected from the group consisting of H, alkyl, substituted alkyl, and alkoxyl; and

R, R₁, R₂, R₃ and R₄ can each independently be present or absent and if present each is independently selected from the group consisting of H, alkyl, substituted alkyl, alkenyl, substituted alkenyl, alkynyl, substituted alkynyl, aryl and substituted aryl;

or a pharmaceutically acceptable salt thereof.

In some embodiments, the compound has the general formula (IV): 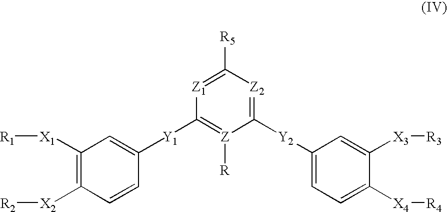
wherein:

X₁, X₂, X₃ and X₄ are each independently selected from the group consisting of H, F, (CH₂)ₙ, CF₂, C═O, O and NR₆, and wherein n is an integer from 1 to 8 and R₆ is selected from the group consisting of H, alkyl, substituted alkyl, and alkoxyl;

Y₁ and Y₂ are each independently selected from the group consisting of C≡C and HC═CH;

Z₁ is selected from the group consisting of CH and N;

Z₂ is selected from the group consisting of CH and N; and

R, R₁, R₂, R₃ R₄ and R₅ can each independently be present or absent and if present each is independently selected from the group consisting of H, alkyl, substituted alkyl, alkenyl, substituted alkenyl, alkynyl, substituted alkynyl, aryl and substituted aryl;

or a pharmaceutically acceptable salt thereof.

In some embodiments, the bismuth-dithiol complex is comprised of a bismuth(III) ion and a dithiol compound, wherein the dithiol compound has the general formula (V): 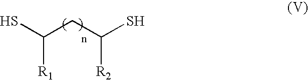
wherein:

n is an integer from 0 to 2; and

R₁ and R₂ are each independently selected from the group consisting of H, alkyl, substituted alkyl, alkenyl, substituted alkenyl, alkynyl, substituted alkynyl, aryl and substituted aryl, or

R₁ and R₂ can together form a five-, six- or seven-membered cycloalkyl, substituted cycloalkyl, heterocyclic, substituted heterocyclic, aryl, or substituted aryl ring structure;

or a pharmaceutically acceptable salt thereof; subject to the proviso that the compound of formula (V) is not 2,3-dimercapto-1-propanol.

In still another embodiment of the presently disclosed subject matter, a method for identifying compounds that modulate RecA protein activity is provided. In some embodiments, the method comprises contacting a candidate compound with a RecA protein, and determining whether the candidate compound modulates the activity of the RecA protein. In some embodiments, determining whether the candidate compound modulates the activity of the RecA protein comprises measuring the interference of assembly of monomeric RecA protein subunits into a nucleoprotein filament when the candidate compound contacts the RecA protein, which in some embodiments comprises measuring the amount of monomeric RecA protein subunits released from the nucleoprotein filament. In other embodiments, determining whether the candidate compound modulates the activity of the RecA protein comprises measuring inhibition of adenosine triphosphate (ATP) hydrolysis by the RecA protein, which in some embodiments comprises measuring the decrease in production of phosphate resulting from inhibition of the RecA protein ATP hydrolysis by the candidate compound. In some embodiments, measuring inhibition of ATP hydrolysis by the RecA protein comprises measuring the decrease in the production of ADP resulting from inhibition of the RecA protein ATP hydrolysis by the candidate compound. In some embodiments, determining whether the candidate compound modulates the activity of the RecA protein comprises measuring the interference of assembly of monomeric RecA protein subunits into a nucleoprotein filament when the candidate compound contacts the RecA protein and measuring inhibition of adenosine triphosphate (ATP) hydrolysis by the RecA protein.

In still another embodiment of the presently disclosed subject matter, a method of inhibiting RecA protein activity in a bacterium is provided, comprising contacting the RecA protein with a compound that interferes with assembly of monomeric RecA protein subunits into a nucleoprotein filament. In some embodiments, the compound is a mimetic of the N-terminal helical domain of the RecA protein, and in some embodiments, the N-terminal helical domain of the RecA protein comprises amino acid residues 1-31 of E. coli RecA protein. Further, in some embodiments, the compound comprises the amino acid sequence B—X₃-Z-X₂-Z-Z-X₂-Z-X₃-Z, wherein B is lysine or arginine; Xₙ is n number of any amino acids and X can be the same or different amino acids; and Z is alanine, valine, leucine, isoleucine, phenylalanine, or methionine. In some embodiments, the compound is selected as a mimetic of the N-terminal helical domain of the RecA protein utilizing an mRNA display assay.

In another embodiment of the presently disclosed subject matter, a method of inhibiting RecA protein activity in a bacterium is provided comprising contacting the RecA protein with a compound that interferes with adenosine triphosphate hydrolysis by the RecA protein. In some embodiments, the compound is selected from the group consisting of a modified adenosine, a modified 5-propynyl-deoxyuridine, a curcumin derivative and a bismuth-dithiol complex. In some embodiments, the compound is a modified adenosine selected from the group consisting of a modified adenosine monophosphate, a modified adenosine diphosphate and a modified adenosine triphosphate. In some embodiments, the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate. In particular embodiments, the compound has the general formula (I), (II), (III), (IV), or (V).

In still another embodiment of the presently disclosed subject matter, a method of treating a bacterial infection in a subject is provided. In some embodiments, the method comprises administering to the subject a therapeutically effective amount of a pharmaceutical composition comprising a compound that modulates bacterial RecA protein activity. In some embodiments, the compound modulates RecA activity by interfering with assembly of monomeric RecA protein subunits into a nucleoprotein filament. In some embodiments, the compound is a mimetic of the N-terminal helical domain of the RecA protein, and in some embodiments, the N-terminal helical domain of the RecA protein comprises amino acid residues 1-31 of E. coli RecA protein. Further, in some embodiments, the compound comprises the amino acid sequence B—X₃-Z-X₂-Z-Z-X₂-Z-X₃-Z, wherein B is lysine or arginine; Xₙ is n number of any amino acids and X can be the same or different amino acids; and Z is alanine, valine, leucine, isoleucine, phenylalanine, or methionine. In other embodiments, the compound modulates RecA activity by interfering with adenosine triphosphate hydrolysis by the RecA protein. In some embodiments, the compound is selected from the group consisting of a modified adenosine, a modified 5-propynyl-deoxyuridine, a curcumin derivative and a bismuth-dithiol complex. In some embodiments, the compound is a modified adenosine selected from the group consisting of a modified adenosine monophosphate, a modified adenosine diphosphate and a modified adenosine triphosphate. In some embodiments, the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate. In particular embodiments, the compound has the general formula (I), (II), (III), (IV), or (V). In some embodiments, the pharmaceutical composition further comprises an antibiotic, and in some embodiments the antibiotic is a replication inhibitor. In some embodiments, the replication inhibitor is selected from the group consisting of actinomycins, adriamycin, aflatoxins, altromycins, anthramycin, bleomycins, calicheamicins, carmustine (BCNU), daunomycin, distamycins, dynemicins, echinomycin, esperamicins, kericidin, mitomycins, neocarzinostatin, netropsins, nitric oxide, nitrogen mustards, nitrosamines, peroxides, pluramycins, pyrrolo[1,4]benzodiazepines, sibiromycin, streptozotocin, tomamycin, beta-lactams, quinolones, fluoroquinolones, DNA Gyrase inhibitors, DNA Polymerase I inhibitors, nucleoside and nucleotide analogs, ribonucleotide reductase inhibitors, antifolates, and DNA biosynthesis inhibitors.

In another embodiment of the presently disclosed subject matter, a method for impeding development of resistance to an antibiotic by a bacterium is provided. In some embodiments, the method comprises contacting the bacterium with a composition comprising the antibiotic and a compound that modulates activity of a RecA protein of the bacterium. In some embodiments, modulating the activity of the RecA protein comprises interfering with assembly of monomeric RecA protein subunits into a nucleoprotein filament. In some embodiments, the compound is a mimetic of the N-terminal helical domain of the RecA protein, and in some embodiments the N-terminal helical domain of the RecA protein comprises amino acid residues 1-31 of E. coli RecA protein. Further, in some embodiments, the compound comprises the amino acid sequence B—X₃-Z-X₂-Z-Z-X₂-Z-X₃-Z, wherein B is lysine or arginine; Xₙ is n number of any amino acids and X can be the same or different amino acids; and Z is alanine, valine, leucine, isoleucine, phenylalanine, or methionine. In other embodiments, modulating the activity of the RecA protein comprises interfering with adenosine triphosphate hydrolysis by the RecA protein. In some embodiments, the compound is selected from the group consisting of a modified adenosine, a modified 5-propynyl-deoxyuridine, a curcumin derivative and a bismuth-dithiol complex. In some embodiments, the compound is a modified adenosine selected from the group consisting of a modified adenosine monophosphate, a modified adenosine diphosphate and a modified adenosine triphosphate. In some embodiments, the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate. In particular embodiments, the compound has the general formula (I), (II), (III), (IV), or (V). In some embodiments the antibiotic is a replication inhibitor. In some embodiments, the replication inhibitor is selected from the group consisting of actinomycins, adriamycin, aflatoxins, altromycins, anthramycin, bleomycins, calicheamicins, carmustine (BCNU), daunomycin, distamycins, dynemicins, echinomycin, esperamicins, kericidin, mitomycins, neocarzinostatin, netropsins, nitric oxide, nitrogen mustards, nitrosamines, peroxides, pluramycins, pyrrolo[1,4]benzodiazepines, sibiromycin, streptozotocin, tomamycin, beta-lactams, quinolones, fluoroquinolones, DNA Gyrase inhibitors, DNA Polymerase I inhibitors, nucleoside and nucleotide analogs, ribonucleotide reductase inhibitors, antifolates, and DNA biosynthesis inhibitors.

In yet another embodiment of the presently disclosed subject matter, a method of enhancing antimicrobial activity of an antibiotic is provided. In some embodiments, the method comprises contacting a bacterium with the antibiotic and a compound that modulates activity of a RecA protein of the bacterium.). In some embodiments the antibiotic is a replication inhibitor. In some embodiments, the replication inhibitor is selected from the group consisting of actinomycins, adriamycin, aflatoxins, altromycins, anthramycin, bleomycins, calicheamicins, carmustine (BCNU), daunomycin, distamycins, dynemicins, echinomycin, esperamicins, kericidin, mitomycins, neocarzinostatin, netropsins, nitric oxide, nitrogen mustards, nitrosamines, peroxides, pluramycins, pyrrolo[1,4]benzodiazepines, sibiromycin, streptozotocin, tomamycin, beta-lactams, quinolones, fluoroquinolones, DNA Gyrase inhibitors, DNA Polymerase I inhibitors, nucleoside and nucleotide analogs, ribonucleotide reductase inhibitors, antifolates, and DNA biosynthesis inhibitors. In some embodiments, modulating the activity of the RecA protein comprises interfering with assembly of monomeric RecA protein subunits into a nucleoprotein filament. In some embodiments, the compound is a mimetic of the N-terminal helical domain of the RecA protein, and in some embodiments the N-terminal helical domain of the RecA protein comprises amino acid residues 1-31 of E. coli RecA protein. Further, in some embodiments, the compound comprises the amino acid sequence B—X₃-Z-X₂-Z-Z-X₂-Z-X₃-Z, wherein B is lysine or arginine; Xₙ is n number of any amino acids and X can be the same or different amino acids; and Z is alanine, valine, leucine, isoleucine, phenylalanine, or methionine. In other embodiments, modulating the activity of the RecA protein comprises interfering with adenosine triphosphate hydrolysis by the RecA protein. In some embodiments, the compound is selected from the group consisting of a modified adenosine, a modified 5-propynyl-deoxyuridine, a curcumin derivative and a bismuth-dithiol complex. In some embodiments, the compound is a modified adenosine selected from the group consisting of a modified adenosine monophosphate, a modified adenosine diphosphate and a modified adenosine triphosphate. In some embodiments, the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate. In particular embodiments, the compound has the general formula (I), (II), (III), (IV), or (V).

In another embodiment of the presently disclosed subject matter, a composition comprising a compound that modulates activity of a RecA protein of the bacterium and a carrier is provided. In some embodiments, modulating the activity of the RecA protein comprises interfering with assembly of monomeric RecA protein subunits into a nucleoprotein filament. In some embodiments, the compound is a mimetic of the N-terminal helical domain of the RecA protein, and in some embodiments the N-terminal helical domain of the RecA protein comprises amino acid residues 1-31 of E. coli RecA protein. Further, in some embodiments, the compound comprises the amino acid sequence B—X₃-Z-X₂-Z-Z-X₂-Z-X₃-Z, wherein B is lysine or arginine; Xₙ is n number of any amino acids and X can be the same or different amino acids; and Z is alanine, valine, leucine, isoleucine, phenylalanine, or methionine. In other embodiments, modulating the activity of the RecA protein comprises interfering with adenosine triphosphate hydrolysis by the RecA protein. In some embodiments, the compound is selected from the group consisting of a modified adenosine, a modified 5-propynyl-deoxyuridine, a curcumin derivative and a bismuth-dithiol complex. In some embodiments, the compound is a modified adenosine selected from the group consisting of a modified adenosine monophosphate, a modified adenosine diphosphate and a modified adenosine triphosphate. In some embodiments, the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate. In particular embodiments, the compound has the general formula (I), (II), (III), (IV), or (V). In some embodiments, the composition further comprises an antibiotic and in some embodiments the antibiotic is a replication inhibitor. In some embodiments, the replication inhibitor is selected from the group consisting of actinomycins, adriamycin, aflatoxins, altromycins, anthramycin, bleomycins, calicheamicins, carmustine (BCNU), daunomycin, distamycins, dynemicins, echinomycin, esperamicins, kericidin, mitomycins, neocarzinostatin, netropsins, nitric oxide, nitrogen mustards, nitrosamines, peroxides, pluramycins, pyrrolo[1,4]benzodiazepines, sibiromycin, streptozotocin, tomamycin, beta-lactams, quinolones, fluoroquinolones, DNA Gyrase inhibitors, DNA Polymerase I inhibitors, nucleoside and nucleotide analogs, ribonucleotide reductase inhibitors, antifolates, and DNA biosynthesis inhibitors. In some embodiments, the carrier is a pharmaceutically acceptable carrier and in some embodiments the carrier is pharmaceutically acceptable in humans.

Accordingly, it is an object of the presently disclosed subject matter to provide inhibitors of RecA activities for control of antibiotic-resistant bacteria. This object is achieved in whole or in part by the presently disclosed subject matter.

An object of the presently disclosed subject matter having been stated above, other objects and advantages will become apparent to those of ordinary skill in the art after a study of the following description of the presently disclosed subject matter and non-limiting examples.

### BRIEF DESCRIPTION OF THE DRAWINGS

FIG. 1, top panel, is a schematic drawing showing inhibitors of RecA activities—ATP analogs, peptides, and bismuth-dithiol complexes. FIG. 1, bottom left panel, is a graph showing mitomycin C, a known genotoxin whose effects are magnified in recA⁻ cells, with hypothetical survival curves indicating that IRAs can produce synergistic antibiotic effects. FIG. 1, bottom right panel, is a graph showing antibiotic resistance to ciprofloxacin, a known replication inhibitor, with hypothetical survival curves indicating that IRAs can produce synergistic antibiotic effects. These growth curves indicate that IRAs can retard the development of antibiotic resistance.

FIG. 2 is a model showing structural representations of RecA intermonomer contacts. The left panel shows a RecA trimer with middle monomer shown as ribbons. Note N-terminal domain (top ribbon) interacting with cleft on upper monomer. The right panel shows a close view of key hydrophobic residues at the interface between one monomer (left ribbon) and the N-terminal domain of another monomer (right ribbon).

FIGS. 3a and 3b are bar graphs showing relative stabilities of NPFs formed in the presence or in absence of nucleotide as measured by the salt titration midpoint (FIG. 3a) and relative inhibition of NPF formation in the presence of various nucleoside di- and triphosphates (FIG. 3b).

FIG. 4 is a radiograph showing binding of RecA protein synthesized in vitro to ^{B}RecA-SA-PMP. 35S-Labeled RecA protein was synthesized in vitro and the unfractionated translation reaction was applied to ^{B}RecA-SA-PMP and washed with increasing NaCl concentrations as shown. The column was treated with GdnHCl (5 M) to remove retained RecA protein. Column fractions were analyzed by SDS-PAGE.

FIG. 5 shows selection profiles for the first four rounds using ^{B}RecA-SA-PMP and mRNA-displayed random peptide library (³⁵S-labeled). Fraction 1 is the supernatant after mixing the library with immobilized RecA. Fractions 2-5 are from 0.8 M NaCl washes (supernatant) following loading. Fractions 6-8 are from 2 M NaCl washes. Fraction 9 is from mRNA-peptide fusions that remain on the beads. The maximal scale for Z axis is shown only at 15% to reveal small changes in fraction 6.

FIGS. 6A-6C are schematic designs and related peptide sequences of proteins that mimic the RecA N-terminal domain.

FIG. 7 is a series of graphs showing development of chloramphenicol resistance in recA⁻ bacteria is significantly delayed (FIG. 7, upper panel). “Rate” of resistance development (reciprocal of mean latency times) correlates with level SOS mutagenesis (FIG. 7, lower panel).

FIG. 8 is a graph showing influence of ADP analogs 1-3 on SOS induction in permeabilized E. coli.

FIG. 9 is a series of graphs showing influence of BiBAL on level of SOS induction following MMC treatment (FIG. 9, upper panel). Synergistic antibiotic effects of BiBAL and MMC are shown in FIG. 9, lower panel.

FIG. 10 is a schematic design showing the structure of a RecA dimer highlighting intermolecular contacts of the N-terminal helix and the filamentation area. The close-up (right side box) shows the expected contact area of the INPEP designs. The specific sequences of the two INPEP designs are seen at the bottom. Open box indicates α-helical region and the shaded box indicates β-sheet. Important features are noted.

FIGS. 11A and 11B show results of INPEP-1 peptide series inhibition of RecA activity. FIG. 11A is a graph showing dose-dependent inhibtion of ATPase assay of 1 μM RecA with varying concentrations of INPEP-1-SAc (far right line), rINPEP-1 (middle line), and INPEP-1-TP (far left line). IC₅₀ values calculated by fitting to right hyperbolas are 40, 33, and 5 μM, respectively. FIG. 11B shows SDS-PAGE of assay mixtures at the completion of an ATPase experiment. Covalent modification of RecA by INPEP-1 leads to an electrophoretic mobility shift for RecA (standard, center lane).

FIG. 12 is a schematic design and initial results of INPEP-2. Energy minimized structure of INPEP-2 with monomethyl arsenic(III) (baII) bound to chelating cysteines at the C-term of the helix to make the “As(III) staple”. Initial ATPase studies revealed that rINPEP-2 (black) is roughly 10-fold more efficacious than rINPEP-1 (gray), with an estimated IC₅₀ of 4 μM.

### DETAILED DESCRIPTION

The presently disclosed subject matter will now be described more fully hereinafter with reference to the accompanying Drawings and Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist.

The continued availability of effective antibiotic therapy appears to depend on the continuous development of new antibiotics. However, only one new class of antibiotic has been introduced in the past 40 years. Although several new antibiotics are under development, these only represent modifications of existing drugs (Livermore, 2004). Indeed, many pharmaceutical companies have either terminated, or greatly reduced, their in-house antibiotic discovery programs. The presently disclosed subject matter provides a new approach to solving this problem, specifically through reducing or preventing the development of mutations in bacteria that lead to antibiotic resistance.

Antibiotics exert enormous selection pressure and stress on a bacterium, leading to the induction of the SOS response and adaptive mutation. Moreover, the gene products induced during SOS, including RecA, participate in the recombinational events that allow horizontal gene transfer by transformation or conjugation. Abrogating these processes can reduce the rate of evolution of resistance. Thus, the presently disclosed subject matter provides compounds for inhibiting RecA activity and methods of using same to reduce development of antibiotic resistance in bacteria and enhance antimicrobial activity of antibiotics.

### I. GENERAL CONSIDERATIONS

I.A. Origins of antibiotic resistance and its spread between bacteria

Drug resistance not only arises quickly, it spreads very rapidly because bacteria may acquire resistance to antimicrobials to which they were previously susceptible through a variety of genetic mechanisms. The most threatening are those that involve resistance genes that are part of mobile genetic elements, such as plasmids, integrons and prophages, collectively referred to as integrative and conjugative elements (ICEs), which can spread from organism to organism, moving not only between individual bacteria, but also between species and even between bacterial clades. Transfer of resistance can be plasmid-mediated, transferring genetic material directly from plasmids to chromosomes by simple RecA-mediated homologous recombination, or facilitated by transposons. Material can also be transferred horizontally or vertically by plasmids. Natural, in situ transformation, which occurs through direct incorporation of free DNA by bacterial cells, is another RecA-mediated recombination-dependent mechanism by which bacteria acquire resistance (Kaye et al., 2000; Spratt, 1994). Once resistance determinants are carried on the chromosome, they can be transferred directly via vertical clonal dissemination.

I.B. The bacterial RecA Protein: Physiological Roles and Importance

In addition to its well established role in homologous recombination (Cox et al., 2000), a process that allows horizontal gene transfer between species (Radman et al., 2000), the RecA protein (SEQ ID NO: 1; Swissprot Accession No. POA7G6) carries out at least two essential functions. First, RecA initiates and controls the SOS response for DNA damage tolerance via the formation of an nucleoprotein fibers (NPF), which serves as an initiating signal for the derepression of many DNA repair genes (Sassanfar & Roberts, 1990; Courcelle et al., 2001; Janion, 2001; Khil & Camerini-Otero, 2002) and the activation of mutagenic translesion DNA synthesis (Goodman, 2000; Hastings & Rosenberg, 2002). In addition, RecA plays an indispensable role in restarting stalled replication forks, by directly participating in recombinational DNA repair (RDR) (Roca & Cox, 1997). In the latter process, one of the two DNA molecules must have a single-stranded region (either gap or end) to initiate assembly of the nucleoprotein filament (DasGupta et al., 1980; West et al., 1980). This underscores the importance of the nucleoprotein filament (NPF) formation (RecA·ATP·ssDNA) in directing all known RecA functions in vivo as well as in vitro.

I.B.1. The SOS Response to DNA Damage

The SOS response is a set of cellular responses induced by exposure of bacterial cells to a variety of genotoxic or metabolic stresses that damage DNA or interfere with DNA replication. The SOS response includes blocking the cell cycle, global mutagenesis via more than one mechanism, and up-regulation of DNA excision repair and recombination functions. The control elements in the E. coli SOS regulon are the products of genes recA and lexA. When bound to ATP and ssDNA, RecA can stimulate autoproteolysis of the LexA protein. LexA is a repressor that binds to at least 15 different operators dispersed throughout the bacterial genome, thereby regulating the transcription of at least 40 unlinked genes. Most SOS functions are implicated in DNA repair activity, which can be loosely grouped in two categories: elimination (e.g., nucleotide excision repair) and tolerance (e.g., translesion synthesis) of DNA lesions. These activities can restore the original genetic information of generate genetic diversity.

For example, one important SOS activity is translesion DNA synthesis, an activity that is largely responsible for SOS mutagenesis. E. coli possesses three SOS polymerases: PoIIV and POIV (encoded by the dinB gene and umuCD operon, respectively) and PoIII (encoded by the poIB gene). (As yet another example of the importance of RecA to these processes, the autoproteolytic removal of the UmuD pro-peptide is stimulated by the activated RecA nucleoprotein filament.) These enzymes are capable of bypassing the DNA lesions which block primer extension by the replicative DNA polymerases. Translesion synthesis is often performed with high fidelity, but SOS polymerases exhibit much reduced fidelity when they operate on undamaged DNA or on DNA lesions that are not their cognate substrates, thus introducing mutations at a high rate. Indeed, error-prone SOS polymerase-dependent replication is the principal pathway by which ultraviolet light and genotoxic substances stimulate mutagenesis in bacteria.

I.B.2. Adaptive Mutation in Bacteria

For 50 years, neo-Darwinists have held the view that random mutations produce genetic differences among individuals, and selection increases the frequency of advantageous alleles. In 1988, a landmark paper (Cairns et al., 1988) spurred research that has cast doubt on three neo-Darwinian principles: (i) mutations occur independently of the environment, (ii) mutations are due to replication errors, and (iii) mutation rates are constant.

The new paradigm is characterized as “adaptive mutation” in bacteria. The “adaptive” mutations occur when they are selected, in cells that appear not to be dividing, and have been found only in genes whose functions were selected. Adaptive mutation is a mutational program in non-growing cells subjected to starvation and so, like the SOS response, is a temporary mutagenic response to environmental stress. It entails global hypermutation, and the adaptive mutation of several alleles in E. coli is under control of the SOS response. It has been found that genetic recombination enzymes—including RecA—are required for some adaptive mutations. Several of the cellular mechanisms involving SOS and recombination help explain the increased genetic diversity seen under stationary-phase conditions. This new mutation mechanism in non-dividing cells may be important for the development of mutations that cause resistance to antibiotic drugs or lead to pathogenicity of microbes.

I.B.3. Recombinational DNA Repair (RDR)

Another example of an SOS-induced activity is the increase in recombination efficiency effected mostly by overproducing the RecA, RecN, and RuvAB proteins. Such high RDR capacity allows the efficient and error-free repair of double-strand breaks and daughter-strand gaps, but it can also result in chromosomal rearrangements through recombination between partially homologous sequences (Dimpfl & Echols, 1989). Furthermore, the elevated recombination capacity also increases the efficiency of conjugational and transductional recombination, thereby facilitating acquisition of foreign DNA via horizontal DNA transfer (HGT) (Matic et al., 1995). HGT can also be stimulated by the protection of the incoming foreign DNA by the SOS-dependent alleviation of restriction and inhibition of dsDNA exonuclease (ExoV) activity (Hiom & Sedgwick, 1992; Rinken & Wackernagel, 1992).

I.B.4. Other Roles of RecA

In addition to its roles in the response to DNA damage, including SOS induction and RDR, RecA plays other physiological roles that allow the mixing of different gene combinations and the acquisition of new genes. As disclosed hereinabove, natural, in situ transformation, which occurs through direct incorporation of free DNA by bacterial cells, is a RecA-mediated recombination-dependent mechanism by which bacteria acquire resistance. Transformation is especially important for naturally competent organisms such as Neisseria gonorrhoeae, Neisseria meningitidis, Haemophilus inlfuenzae, and Bacillus subtilis.

Some SOS phenomena that are not involved in DNA repair can also increase genetic variability in stressed bacterial populations. Examples of such RecA-dependent processes include the increase in the transposition frequencies of Tn5 and Tn10, and the induction of temperate bacteriophages such as λ, 434, 21, P22, f80, and coliphage 186 (Kuan et al., 1991; Levy et al., 1993; Robey et al., 2001). Although induction of bacteriophage from lysogenic to lytic growth results in cell lysis, the phage can transfer host genes to new recipient cells or allow transformation of new cells by the released chromosomal DNA.

Mobile genetic elements also have a crucial role in spreading antibiotic resistance genes among bacterial populations. As disclosed hereinabove, ICEs are a diverse group of mobile genetic elements that are transferred by means of cell-cell contact and integrate into the chromosome of the new host. It has been recently demonstrated that RecA stimulates an ICE in E. coli and Vibrio cholerae when an active NPF is formed following treatment with the replication inhibitors MMC and ciprofloxacin. This RecA-dependent activation results from NPF-facilitated autoproteolysis of the ICE repressor in an analogous manner to its function in inducing phage k from a lysogen during the SOS response.

I.B.5. Is RecA Necessary for Bacterial Survival?

The functions of the RecA protein are apparently essential for survival of bacterial populations (Cox et al., 2000). There are, however, complexities in the relevant data that require one to keep in mind the difference between the survival needs of an individual and a population of organisms. It is well known that although recA⁻ strains can be aerobically cultured and propagated ex vivo using rich media, mutations in the recA gene significantly reduce the growth rate and viability of Escherichia coli and other eubacteria. Laboratory cultures of recA⁻ strains are composed of three populations of cells: viable cells, nonviable but residually dividing cells, and nonviable and nondividing cells (Capaldo & Barbour, 1975). Nondividing cells are greatly reduced in their ability to synthesize DNA such that they contain little or no DNA. The conclusion that recA⁻ bacteria are not competitive is emphasized by the fact that there are no published reports of recA⁻ populations of eubacteria isolated from natural or in vivo sources. Indeed, comparative genomics has identified recA (or a homolog) as being one of a minimal set of genes that is necessary and sufficient for sustaining a functional cell (Mushegian et al. 1996). Thus, while one may conclude that recA expression is not “essential” for robust aerobic growth in rich media, active RecA protein is clearly necessary for independent survival of a bacterial species under competitive conditions.

I.B.6. recA⁻ Bacteria are Sensitized to Replication Errors

Up to one-half of metabolically active bacterial cells experience replication fork arrest prior to or during cell division. RecA and the accessory RDR machinery are required to repair and restart such stalled replication forks. Bacteria without active RecA protein are therefore especially sensitized to replication errors induced by chemical agents. Indeed, a number of antibacterial agents exert a profoundly more potent effect on recA⁻ cells than on the corresponding wild-type cells, an exemplary number of which are tabulated below (Table 1).

TABLE 1
Selected antibiotics to which recA⁻ bacteria are more susceptible.

| Antibiotic | Target | Effect |
|---|---|---|
| Mitomycin C | DNA | genotoxin |
| Bleomycin | DNA | genotoxin |
| Streptozotocin | DNA | genotoxin |
| Naldixic Acid | GyrA | replication arrest |
| Ciprofloxacin | GyrA/B | replication arrest |
| Microcins B17/D93 | GyrA/B | replication arrest |
| Terpentecin | Pol I | replication arrest |
| 5-Azacytosine | — | replication errors |
| 5-Fluorouracil | — | replication errors |
| AZT | — | replication errors |
| Trimethoprim | FolA | DNA biosynthesis |
| Rifampicin | rpoB | RNA biosynthesis |

I.B.7 Inhibition of RecA Activities

Despite the necessity of RecA activity for the survival of bacterial species and the sensitivity of recA⁻ bacteria to genotoxins and inducers of replication errors, no natural products have thus far been reported to inhibit RecA protein activity. Although a number of natural products were described as abrogating the SOS response, most of these are now known to act by detoxifying genotoxic agents (Pillai et al., 2001). In addition, with the exception of non-hydrolysable analogs of ATP, no synthetic inhibitors of RecA have been reported. It seems reasonable to ask: Why have no natural or non-natural small molecules been reported to inhibit the RecA protein?

The answer to this question is best described as multifaceted, and several issues are described here. First, RecA activity does not appear to be “essential” given the fact that null mutations are viable ex vivo. The relevance of this observation to the absolute necessity for RecA function (and the complexity of the data) was described herein above. Second, RecA has widely been viewed as a “recombination protein” rather than a DNA repair agent based on the historical context of its discovery in 1965 (Clark & Margulies, 1965). The solutions to scientific problems are influenced by the manner in which the questions are framed. In the context of understanding RecA protein function, the framework of ideas influencing genetic recombination science have been largely split into two competing paradigms artificially favoring either recombination or repair. The real biological function of recombination systems were often ignored and, indeed, the true physiological functions of RecA in DNA repair remained enigmatic and were not widely investigated until 30 years after its discovery. Related to this second issue, the third point is that genetic recombination is generally viewed as a complicated or even inscrutable subject. Taken together, these issues have resulted in the complete neglect of RecA inhibition by small molecules.

### II. DEFINITIONS

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein the term “alkyl” refers to C₁₋₂₀ inclusive, linear (i.e., “straight-chain”), branched, or cyclic, saturated or at least partially and in some cases fully unsaturated (i.e., alkenyl (one or more carbon double-bonds) and alkynyl (one or more carbon triple-bonds)) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Alkoxyl” or “alkoxyalkyl” refer to an alkyl-O— group wherein alkyl is as previously described. The term “alkoxyl” as used herein can refer to C₁₋₂₀ inclusive, linear, branched, or cyclic, saturated or unsaturated oxo-hydrocarbon chains, including, for example, methoxyl, ethoxyl, propoxyl, isopropoxyl, butoxyl, t-butoxyl, and pentoxyl.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “amino” refers to the —NH₂ group.

The term “carbonyl” refers to the —(C═O)— group.

The term “carboxyl” refers to the —COOH group.

The terms “halo”, “halide”, or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “keto” refers to a double bonded oxygen group, (═O).

The term “nitro” refers to the —NO₂ group.

The term “thio” refers to a compound described previously herein wherein a carbon or oxygen atom is replaced by a sulfur atom.

The term “sulfate” refers to the —SO₄ group.

When the term “independently selected” is used, the substituents being referred to (e.g., R groups, such as groups R₁ and R₂, or groups X and Y), can be identical or different. For example, both R₁ and R₂ can be substituted alkyls, or R₁ can be hydrogen and R₂ can be a substituted alkyl, and the like.

A named “R”, “R′,” “X,” “Y,” “Y”, “A,” “A′”, “B”, or “Z” group will generally have the structure that is recognized in the art as corresponding to a group having that name, unless specified otherwise herein. For the purposes of illustration, certain representative “R,” “X,” “Y”, and “A” groups as set forth above are defined below. These definitions are intended to supplement and illustrate, not preclude, the definitions that would be apparent to one of ordinary skill in the art upon review of the present disclosure.

As used herein, the term “mimetic” refers to a compound that structurally and/or functionally mimics a target compound. In some embodiments of the presently disclosed subject matter, “mimetic” refers to a RecA mimetic that mimics the structure and/or function of RecA such that an activity of RecA is modulated. For example, a RecA mimetic of the presently disclosed subject matter includes compounds that mimic RecA and thereby interfere with assembly of monomeric RecA subunits into a nucleoprotein filament. In some embodiments, the mimetics of the presently disclosed subject matter comprise peptides.

As used herein, the term “modulate” means an increase, decrease, or other alteration of any, or all, chemical and biological activities or properties of a wild-type or mutant polypeptide, such as a RecA protein. The term “modulation” as used herein refers to both upregulation (i.e., activation or stimulation) and downregulation (i.e. inhibition or suppression) of an activity of the protein.

As used herein, the term “mutation” carries its traditional connotation and means a change, inherited, naturally occurring or introduced, in a nucleic acid or polypeptide sequence, and is used in its sense as generally known to those of skill in the art.

As used herein, the term “nucleoside” is meant to denote a nitrogenous heterocyclic base linked (i.e., via a glycosidic bond) to a pentose sugar, such as but not limited to a ribose, deoxyribose, or derivatives or analogs thereof. The term “nucleoside” as used herein also refers to nucleotides, the phosphoric acid esters of nucleosides which comprising a nitrogenous heterocyclic base, a pentose sugar, and one or more phosphate or modified phosphate group. Nucleotide and nucleoside units may include the common purine and pyrimidine bases such as guanine (G), adenine (A), cytosine (C), thymine (T), uracil (U) or derivatives thereof. Additional purine bases include xanthine, hypoxanthine, theobromine, and caffeine. The pentose sugar may be deoxyribose, ribose, or groups that substitute therefore.

As used herein, the terms “nucleoside analog”, “nucleoside derivative” and “modified nucleoside” are meant to denote nucleoside moieties that have been structurally modified.

As used herein, the terms “polypeptide” and “peptide” mean any polymer comprising any of the 20 protein amino acids, regardless of its size. Each amino acid unit making up the peptide is a “residue.” Thus the term “amino acid residue” refers to the radical or diradical of one of the 20 standard amino acids or of a nonstandard amino acid that results from the loss of a proton from the amine group, the loss of the hydroxyl from the carboxylic acid group, or the loss of both a proton from the amine group and the hydroxyl from the carboxylic acid group. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product.

As used herein, the terms “replication inhibitor” and “replication inhibitor antibiotic” are used interchangeably and refer to genotoxic agents and agents causing metabolic stress that damage or interfere with DNA replication. Exemplary genotoxic agents and DNA-damaging agents include, but are not limited to, actinomycins, adriamycin, aflatoxins, altromycins, anthramycin, bleomycins, calicheamicins, carmustine (BCNU), daunomycin, distamycins, dynemicins, echinomycin, esperamicins, kericidin, mitomycins, neocarzinostatin, netropsins, nitric oxide, nitrogen mustards, nitrosamines (e.g., N-Methyl-N′-Nitro-N-Nitrosoguanidine, or MNNG), peroxides, pluramycins, pyrrolo[1,4]benzodiazepines, sibiromycin, streptozotocin, and tomamycin. Exemplary agents that cause metabolic stress that interferes with DNA replication include, but are not limited to, beta-lactams (e.g., penicillins, cephalosporins, monobactams, and carbapenems), quinolones (e.g., nalidixic acid and pipemidic acid), fluoroquinolones (e.g., ciprofloxacin, norfloxacin, ofloxacin, lomefloxacin, levofloxacin, enoxacin, and sparfloxacin), other DNA Gyrase inhibitors (e.g., microcins), DNA Polymerase I inhibitors (e.g., terpentecin), nucleoside and nucleotide analogs (e.g., AZT), ribonucleotide reductase inhibitors (e.g., hydroxyurea), antifolates (e.g., diaminopyrimidines, such as trimethoprim and trimetrexate, and sulfonamides), and other DNA biosynthesis inhibitors (e.g., 5-azacytosine, 5-fluorouracil, alanosine, hadacidin, azaserine, and DON).

The term “standard amino acid” refers to any of the twenty standard L-amino acids commonly found in naturally occurring peptides. “Nonstandard amino acid” means any amino acid, other than the standard amino acids, regardless of whether it is prepared synthetically or derived from a natural source.

### III. COMPOUNDS FOR MODULATING RecA PROTEIN ACTIVITY

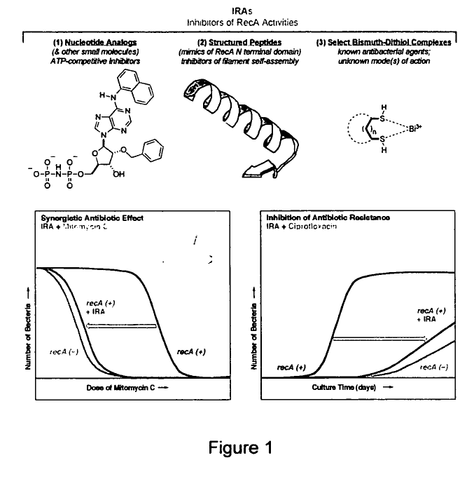

Disclosed herein are compounds that modulate RecA protein activity. In some embodiments, the compound inhibits RecA activity. In some embodiments, the compound that modulates RecA protein activity either interferes with the assembly of monomeric RecA protein subunits into a nucleoprotein filament or interferes with the RecA protein catalyzed adenosine triphosphate (ATP) hydrolysis. Inhibitors of RecA activities (IRAs) could be a powerful additive to conventional antibiotic therapy by increasing the efficacy of antibiotic genotoxins (e.g. Mitomycin C, MMC) and replication inhibitors (ciprofloxacin) and by suppressing the development and spread of drug resistance. See FIG. 1.

III.A. RecA N-terminal Helical Mimetics

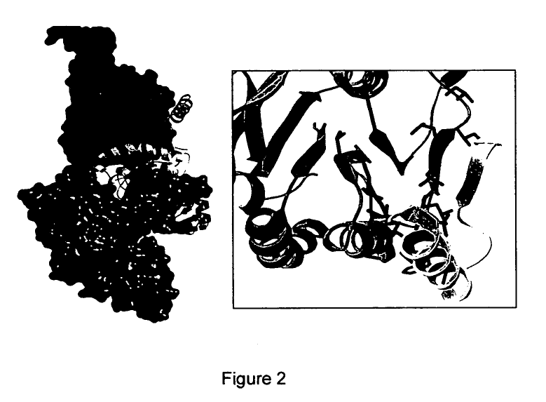

An attractive target for RecA inhibition is the assembly of the activated NPFs capable of SOS induction and RDR. In order to form NPFs, individual monomers must self-assemble in an orderly way. Among the earliest RecA experiments, genetics revealed negative transdominant phenotypes when inactive RecA proteins were co-expressed with wild-type (WT) RecA (Yarranton & Sedgwick, 1982; Larminat, & Defais, 1989; Horii et al., 1992). In particular, deletion mutants expressing RecA truncated at the C-terminus have been found to disrupt RecA functions in the both the SOS response and in the response to DNA damage by ultraviolet radiation (Horii et al., 1992). Such dominant negative phenotypes are diagnostic for protein-protein interactions—in this case between mutant and native RecA proteins—and the subsequent elucidation of RecA filaments provided a structural basis for these observations. The RecA crystal structure demonstrated that the driving force for filament assembly is substantially derived from intermonomer contacts between the helical protrusion at the N-terminus of one monomer with a specific substructure within the core domain of an adjacent monomer (FIG. 2; Story & Steitz, 1992). These monomer-monomer interactions are crucial to the filamentous structure of the RecA nucleoprotein complex and, consequently, for all of its biochemical activities. Hence, the interactions mediated by the N-terminal domain are an attractive target for disruption, as the prevention of monomer-monomer contacts will prevent filament formation and thereby inhibit RecA activity.

The importance of the N-terminal domain, suggested by analysis of the RecA crystal structure, has been corroborated by mutagenesis experiments and comparative protein sequence analysis. The crystal structure of the E. coli RecA protein shows that amino acids 3-21 at the N-terminus are organized in an α-helix, so that amino acid residues 6, 9, 10, 13, 14, 17, 20 and 21 are exposed on a surface involved in the interaction with adjacent monomers within the RecA nucleoprotein filament (Storv & Steitz, 1992). In agreement, deletion of the first N-terminal 33 amino acids of RecA protein (ΔN33 RecA) abolishes its ssDNA binding and ATP hydrolysis activities (Mikawa et al., 1995). Kowalczykowski and coworkers subsequently demonstrated that all N-terminal deletions of more than five amino acids manifest a RecA⁻ phenotype (Zaitsev & Kowalczykowski, 1998). Subsequently, site-directed mutagenesis of residues S25, I26, and L29 revealed that small changes in the identity of specific sidechains could profoundly affect RecA's recombinogenic properties (Chervyakova et al., 2001).

Provided herein for this first time is the disruption of stable monomer associations in the N-terminal domain in RecA function. In some embodiments the disruption of such associations is provided via peptides that mimic the structure of the N-terminal domain, affording a highly specific method of RecA inhibition.

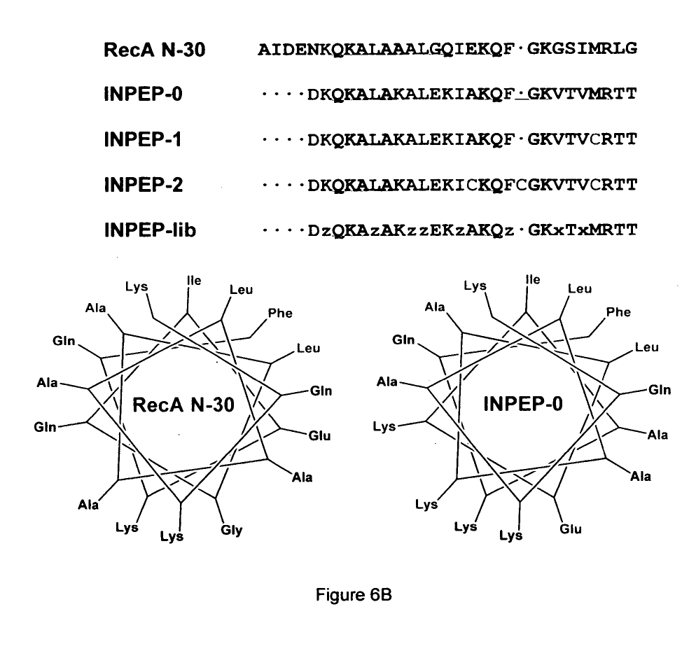

In some embodiments of the method, the compound that contacts the RecA protein is one that can interfere with assembly of monomeric RecA protein subunits into a nucleoprotein filament (NPF). For example, in some embodiments, the compound is a mimetic of the N-terminal helical domain of the RecA protein. A non-limiting exemplary N-terminal helical domain of a RecA protein includes amino acid residues 1-31 of E. coli RecA protein (e.g., SEQ ID NO:2). However, the presently disclosed subject matter is not intended to be limited only to mimetics of E. coli N-terminal helical domain, but rather is inclusive of RecA N-terminal helical domains from other bacteria as well. For example, Yersinia pestis, Salmonella typhi, Shigella sonnei, and Proteus vulgaris each comprise RecA proteins having an N-terminal helical domain identical to that of E. coli. Numerous other bacteria known in the art express RecA proteins having N-terminal domains homologous to the sequences disclosed herein, and are therefore intended as well to be encompassed by the presently disclosed subject matter. As such, in some embodiments, the N-terminal helical domain mimetic compound comprises the amino acid sequence B—X₃-Z-X₂-Z-Z-X₂-Z-X₃-Z (SEQ ID NO:3), wherein B is lysine or arginine; Xₙ is n number of any amino acids and X can be the same or different amino acids; and Z is alanine, valine, leucine, isoleucine, phenylalanine, or methionine. Further, in some embodiments, the N-terminal helical domain mimetic compound comprises the amino acid sequence DKQKALAKALEKIAKQFGKVTVMRTT (SEQ ID NO:8), DKQKALAKALEKIAKQFGKVTVCRTT (SEQ ID NO:9), or DKQKALAKALEKICKQFCGKVTVCRTT (SEQ ID NO:10). See FIG. 6B. A compound that “mimics” or is a “mimetic” the N-terminal helical domain of RecA is taken to mean a compound that resembles the N-terminal helical domain of a naturally-occurring N-terminal helical domain in shape (e.g., three-dimensional structure) and/or function.

Determining suitable amino acid sequences for RecA N-terminal mimetics can be based on rational design or screening libraries of random peptides, for example random 20 mers or random 30 mers. Additionally, determining possible amino acid sequences for RecA N-terminal mimetics can be based on a “peptide grafting” approach, for example, a developed by the Schepartz laboratory (Zondlo & Schepartz, 1999).

Peptide grafting is a general strategy for the design of miniature proteins that present a solvent-exposed α-helix able to recognize their biomolecular targets with high affinity and specificity (Sia & Kim, 2003; Shimba et al., 2004). For example, following the approach of Zondlo and Schepartz, the design process can begin with avian pancreatic polypeptide (aPP), a small, well-folded protein of known structure including a single α-helix stabilized by hydrophobic interaction with a type II polyproline helix. Since formation of the aPP core requires residues on only one face of the aPP α-helix (shown as light shaded letters in FIG. 6A) the opposite, solvent-exposed face of the α-helix is available for recognition of the core domain of a RecA monomer. By grafting various combinations of those six residues (shown as darker shaded letters in FIG. 6A and 6C) used by RecA to form filaments on the solvent-exposed a-helical face of aPP, a 30 mer peptide (aPP-RecA Graft, SEQ ID NO:4) was generated containing all of the self-interaction contact residues of RecA and all of the folding residues of aPP (FIG. 6A). As controls, a 30 mer aPP sequence (without grafting any residues from RecA; aPP-30, SEQ ID NO:5) and the N-terminal 30 amino acids of RecA (RecA N-30, SEQ ID NO:6) were also synthesized.

The ability of each of these three peptides to disrupt the ATPase activity of the RecA NPF was tested. The results show that the peptide designed by grafting shows little ability to inhibit RecA (/₅₀>1.5 mM), although the RecA N-terminal peptide (/₅₀=600 μM) and aPP itself (/₅₀=150 μM) are somewhat stronger inhibitors.

Additional peptides can be constructed using this approach, and a library of partially randomized compounds generated and screened. See FIG. 6C. Thus, the compounds encompassed by FIGS. 6A-6C are candidates for the development of RecA N-terminal mimetics

In general, natural peptides larger than four or five amino acids in size are not actively transported into bacteria by the oligopeptide transport machinery. Special sequences that confer bacterial cell permeability can, however, be directly incorporated into synthetic peptides (Good et al., 2001; Rajarao et al., 2002). Several oligopeptide sequences have been reported that allow transport of peptides, proteins, and PNAs into bacterial cells (Good et al., 2001; Rajarao et al., 2002). Thus the sequence CFFKDEL (SEQ ID NO:7)—recently show to deliver GFP in E. coli (Raiarao et al., 2002)—can be appended to the N-terminus of the RecA-inhibiting peptides. An alternative strategy to increase cell permeability of RecA N-terminal mimetics is the direct expression of the desired peptide from a recombinant plasmid, for example the mimetics can be expressed fused to a well-characterized soluble protein (e.g., maltose binding protein (Kapust & Wauqh, 1999) with an intervening self-cleaving intein) (Singleton et al., 2002).

III.B. Small Molecule RecA Inhibitors

Many ATPase inhibitors have been described, and several are already on the market or are in clinical trials (Chene, 2002). Yet, although a number of selective ATPase inhibitors have been described, none of them binds to the ATP-binding site. The design of competitive ATP inhibitors represents a new way of targeting ATPases. To show in vivo activity, such molecules should be able to compete with the high ATP concentration that is present in the cell (2-10 mM), and should be highly selective, because of the similarity of the ATP-binding site among ATPases and other ATP-binding proteins. This approach has been carried out successfully with another family of ATP-binding proteins: the protein kinases (Al-Obeidi & Lam, 2000; Cohen, 2002; Scapin, 2002).

The presently disclosed subject matter provides several classes of small molecules that can interfere with the ability of RecA to hydrolyze ATP. In some embodiments, the compound that interferes with the ability of RecA to hydrolyze ATP is a modified adenosine, a modified 5-propynyl-deoxyuridine, a curcumin derivative or a bismuth-dithiol complex.

III.B.1. N⁶-Modified Adenosine Derivatives

In spite of its gross morphological similarities with the other proteins' ATP-binding domains, the RecA protein displays a unique characteristic in that it binds ADP with the adenine ring flipped into a new location. As a result of ADP's unusual relative orientation, the adenosine moiety in RecA is located in a wide crevice near the surface of the protein rather than having adenine buried in a hydrophobic pocket as in other ATP-binding proteins (Chene, 2002; Mao et al., 2004). Hence, it appears that the ATP-binding site of RecA could uniquely accommodate a sterically demanding substituent at N⁶ of adenine. In contrast, the ATP-binding sites of other NTPases and kinases maintain close contact with the edge and both faces of adenine, mitigating their potential interactioris with N6-substituted adenosine nucleotides (Shah et al.,1997; Gillespie et al., 1999; Kapoor & Mitchison, 1999; Chene, 2002; Mao et al., 2004). An initial approach to developing small molecule inhibitors of RecA thereby relies on the principles of negative design, i.e., the achievement of specificity through the introduction of substituents that prevent the formation of non-productive alternative complexes with non-targeted enzymes.

Further analysis of the RecA molecular surface reveals an internal cavity in the back of its nucleotide-binding pocket. Interestingly, this cavity is located in the same region as that in which the adenine ring of ADP appears in the crystal structures of every other ATPase. This appears to be a unique structural feature of the RecA·ADP complex. This additional difference between RecA and other P-loop ATPases can potentially be exploited using NDP analogs bearing 2′/3′ substituents that can be accommodated in the unique hydrophobic pocket. Some of the variations of NDP analogs that can be tested as potential RecA inhibitors are shown below in Scheme 1.

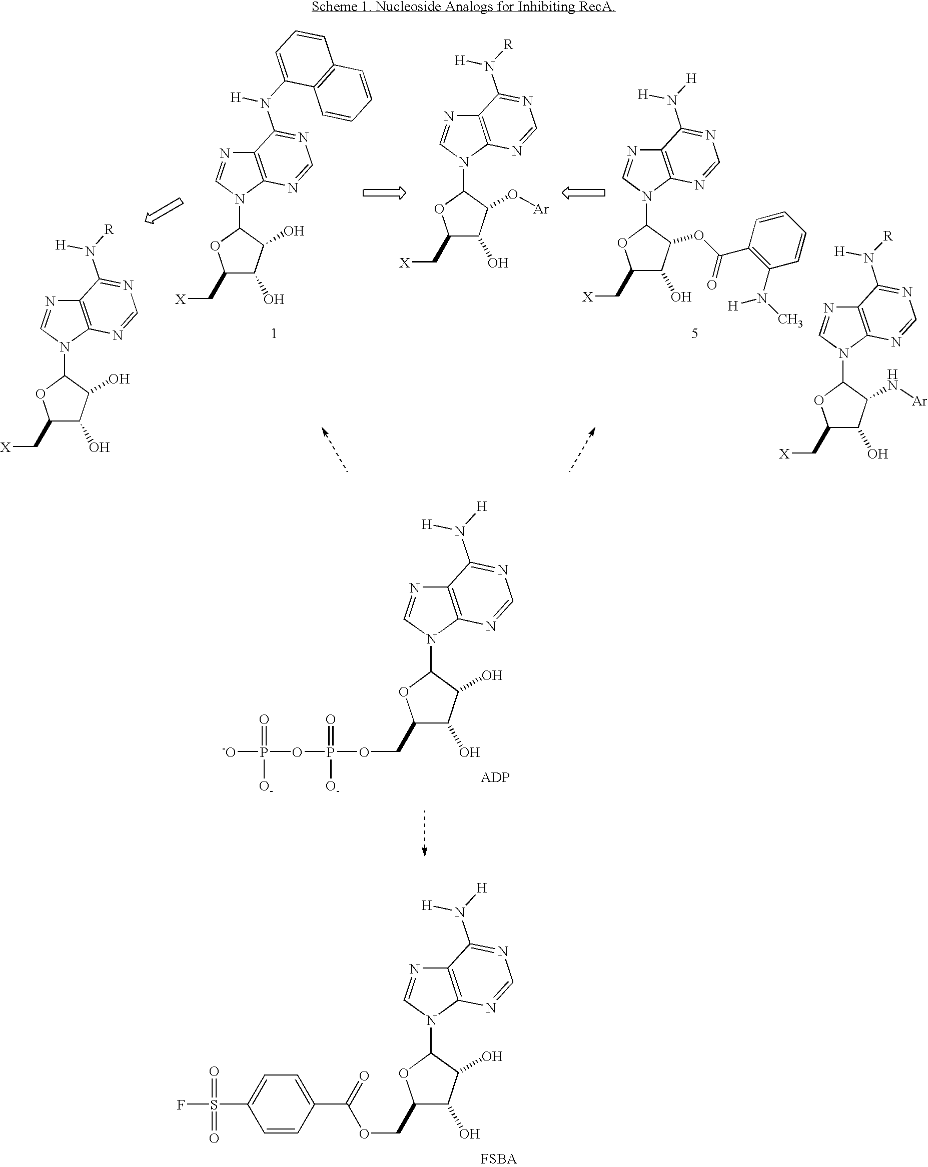

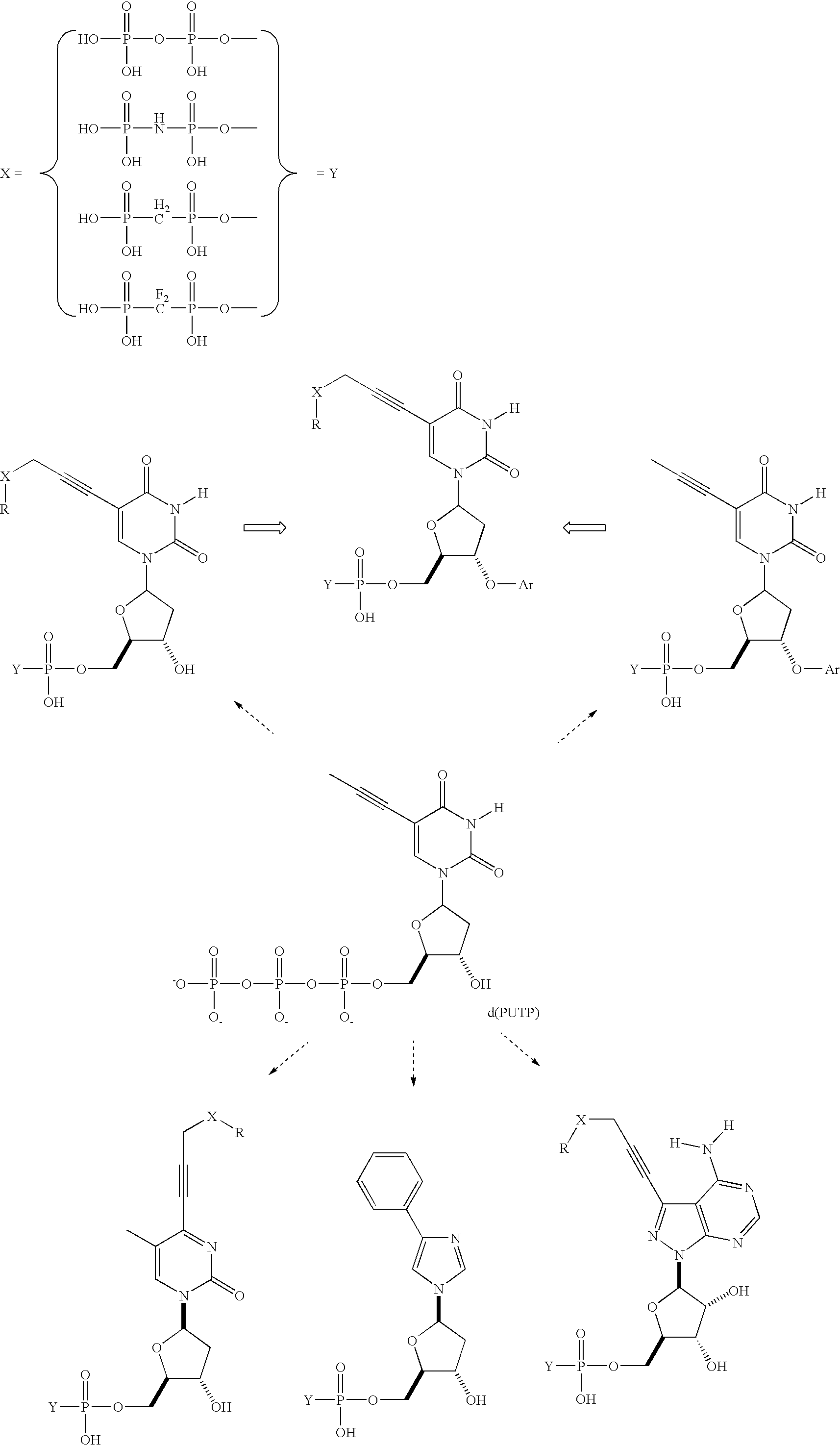

The term “modified adenosine derivative” as used herein refers to compounds that comprise the purine base adenine wherein the adenine amino group can be substituted with an alkyl, aryl or aralkyl substituent. Modified adenosines of the presently disclosed subject matter include modified adenosine monophosphates, modified adenosine diphosphates and modified adenosine triphosphates. In some embodiments, the modified adenosine derivative comprises a phosphate isostere, a moiety that is titratable at physiological pH and, thus, could be neutral prior to and during cell penetration, but would become ionic in the cell to better interact with the RecA ATP site.

In some embodiments, provided is a compound of Formula (I): 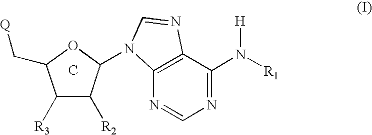
wherein:

R₁ is selected from the group consisting of: 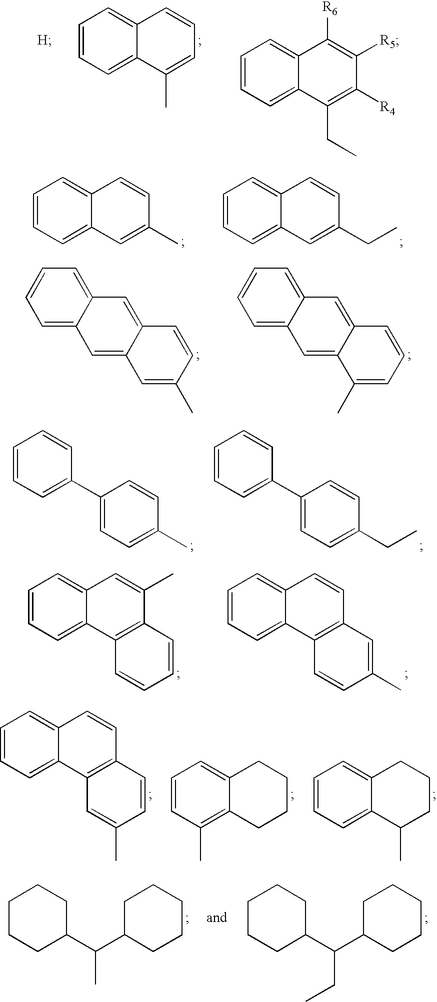
wherein R₄, R₅ and R₆ are each independently selected from the group consisting of H, alkyl, substituted alkyl, OH, alkoxyl, and substituted alkoxyl;

R₂ and R₃ are each independently selected from the group consisting of H, F, OH, NH₂ and Y-Z-R₇,

wherein Y is selected from the group consisting of O and NR₂₂, and wherein R₂₂ is selected from the group consisting of H, alkyl, substituted alkyl, and alkoxyl;

Z is selected from the group consisting of (CH₂)ₚ, CF₂ and C═O, and wherein p is an integer from 1 to 8; and

R₇ is selected from the group consisting of H, alkyl, substituted alkyl, alkenyl, substituted alkenyl, alkynyl, substituted alkynyl, aryl and substituted aryl, or

R₂ and R₃ can together with ring C form the following five-membered heterocyclic ring structure: 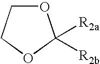
wherein R₂ₐ and R_{2b} are each independently selected from the group consisting of H, alkyl, substituted alkyl, alkenyl, substituted alkenyl, alkynyl, substituted alkynyl, aryl and substituted aryl; and

Q is selected from the group consisting of: 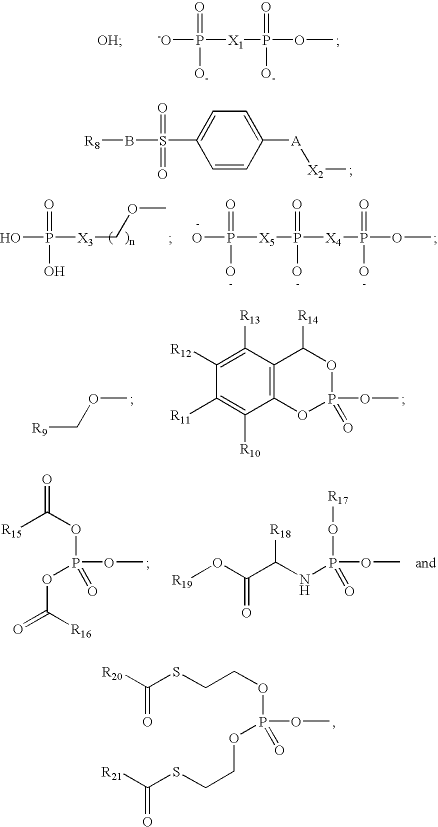
wherein X₁, X₂, X₃, X₄ and X₅ are each independently selected from the group consisting of O, NR₂₃, CH₂ and CF₂, and wherein R₂₃ is selected from the group consisting of H, alkyl, substituted alkyl, and alkoxyl;

A and B are each independently selected from the group consisting of O, NR₂₄, CH₂, CF₂ and C═O, and wherein R₂₄ is selected from the group consisting of H, alkyl, substituted alkyl, and alkoxyl;

n is an integer from 0 to 4;

R₈ is selected from the group consisting of H, alkyl, substituted alkyl, alkenyl, substituted alkenyl, alkynyl, substituted alkynyl, aryl and substituted aryl;

R₉ is selected from the group consisting of 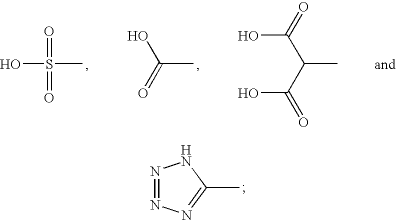
and

R₁₀, R₁₁, R₁₂, R₁₃, R₁₄, R₁₅, R₁₆, R₁₇, R₁₈, R₁₉, R₂₀ and R₂₁ are such independently selected from the group consisting of H, alkyl, substituted alkyl, alkenyl, substituted alkenyl, alkynyl, substituted alkynyl, aryl and substituted aryl;

or a pharmaceutically acceptable salt thereof.

In some embodiments, the compound of Formula (I) is selected from the group consisting of N⁶-(1-napthyl)-adenosine-5′-O-Diphosphate (1), N⁶-(1-benzyl)-adenosine-5′-O-Diphosphate (2), N⁶-(2-phenethyl)-adenosine-5′-O-Diphosphate (3), shown in Scheme 2, below.

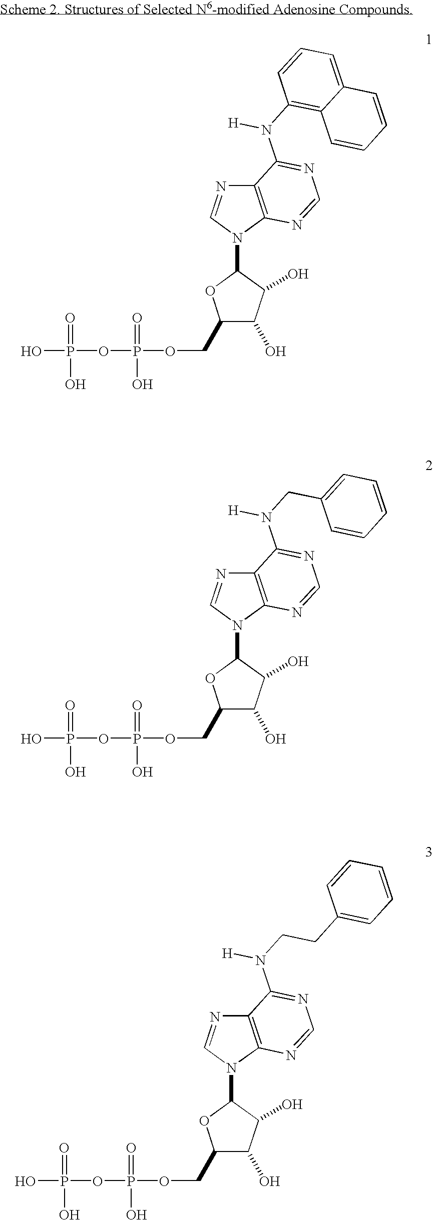

In some embodiments, the modified adenosines are adenosines that are modified at the 2′ and/or 3′ oxygen of the nucleoside sugar. In some embodiments, an N⁶-modified adenosine is further modified at the 2′ and 3′ oxygens of the nucleoside sugar. Indeed, combination of the best N⁶— and 2′(3′) substituents can provide compounds of high affinity and specificity for ATP-competitive inhibition of RecA. Thus, additional compounds of Formula (I) include, but are not limited to, 2′,3′-O—(N-methyl-anthraniloyl)-adenosine-5′diphosphate (mant-ADP) and 2′,(3′)-O-(2,4,6-trinitrophenyl)-adenosine-5′-diphosphate (TNP-ADP) which are both commercially available from Axxora, LLC (San Diego, Calif., United States of America).

Synthesis of additional 2′,(3′)-modified analogs of ADP can be designed to emphasize the differentiation of substituent effects on the 2′ or 3′ positions. In particular, by taking advantage of the ability of dichloro disiloxanes to protect the 3′ and 5′ OH groups simultaneously, one can readily access 2′-O-ethers and 2′-deoxy-2′-amines and -amides (Aronov et al., 1999; Bressi et al., 2000; Bressi 2001). Selective placement of 2′- and 3′-O-esters can be achieved by using 3′- and 2′-deoxyribonucleosides, respectively. Modification of both the N6- and 2′(3′) positions can proceed in a stepwise fashion without complications (Aronov et al., 1999; Bressi et al., 2000; Bressi 2001).

Further, the compounds of Formula (I) can comprise a modified phosphate group at the 5′ O-position of the nucleotide sugar (i.e., variable Q of Formula (I)). In some embodiments, modification of the phosphate group improves the cell permeability of the nucleoside derivative. Compounds of Formula (I) therefore, can contain phosphate ester “prodrugs” of a nucleoside monophosphate wherein the phosphate ester allows for passive transport of the compound into the bacterial cytoplasm. Nucleoside monophosphates can be converted to neutral compounds, for example, by alkylation with a range of substituents (Krise & Stella, 1996). Once inside the bacterial cytoplasm, bacterial enzymes can cleave the alkyl substituent, reconverting the compound to a monophosphate, which can then be transformed by additional bacterial enzymes into an active diphosphate or triphosphate inhibitor. Some embodiments of suitable phosphate modifications are shown below in Scheme 3.

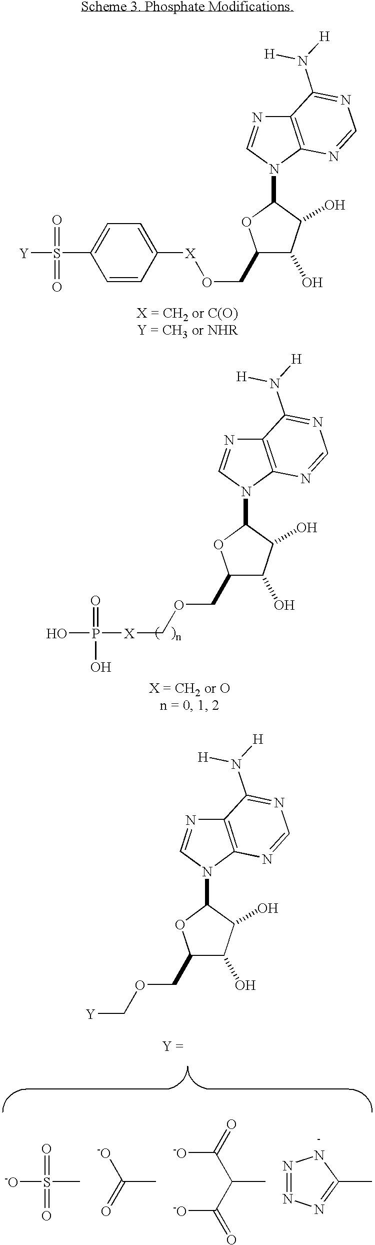

Compounds of Formula (I) can be synthesized via solution-phase techniques as described more fully hereinbelow or by other nucleotide modification techniques as known to one of skill in the art. Further, methods for the automated parallel solid-phase synthesis (SPOS) of nucleosides have been reported in the literature (Hanessian & Huynh, 1999; Epple et al., 2003). Thus, the compounds of Formula (I) may be synthesized using solid-phase methodology, either using a combinatorial or a parallel strategy.

For example, as shown below in Scheme 4, based on the successful use of methoxymethylidene to protect the 2′ and 3′ O atoms of the ribose ring, polymer-bound o-methoxybenzaldehyde (MB-CHO) can be used to immobilize 6-chloropurine riboside. Alkylamine displacement of chloride can then afford a library N⁶-substituted adenosine analogs on solid support. Tosylation (Davisson et al., 1987) or mesylation (Epple et al., 2003; Horwitz et al., 1962) of the 5′-OH would provide the leaving group necessary for subsequent diphosphorylation. Finally, the ADP analogs could be cleaved from the resin using relatively mild acid (Hanessian & Huynh, 1999; Rodenko et al., 2002), or even DDQ (Oikawa et al., 1982).

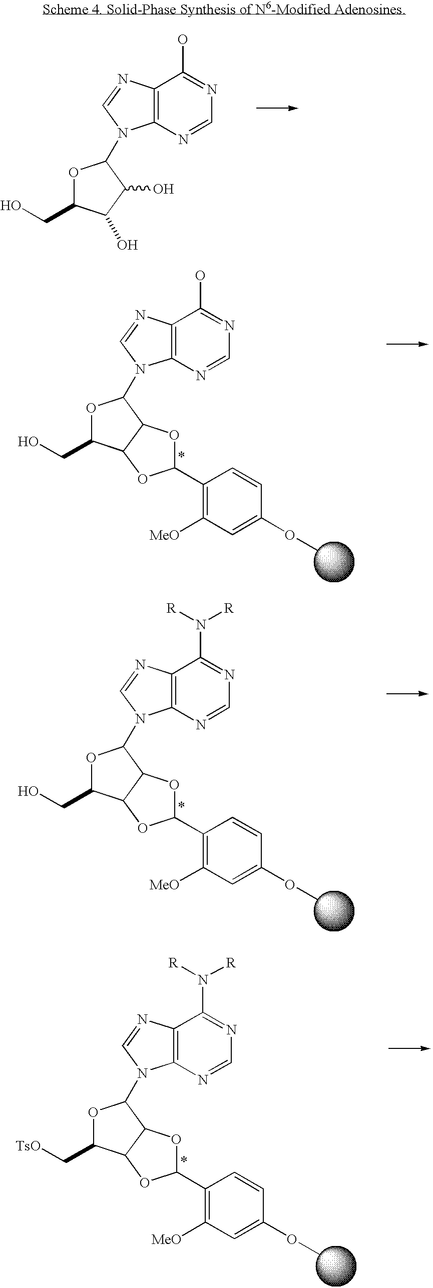

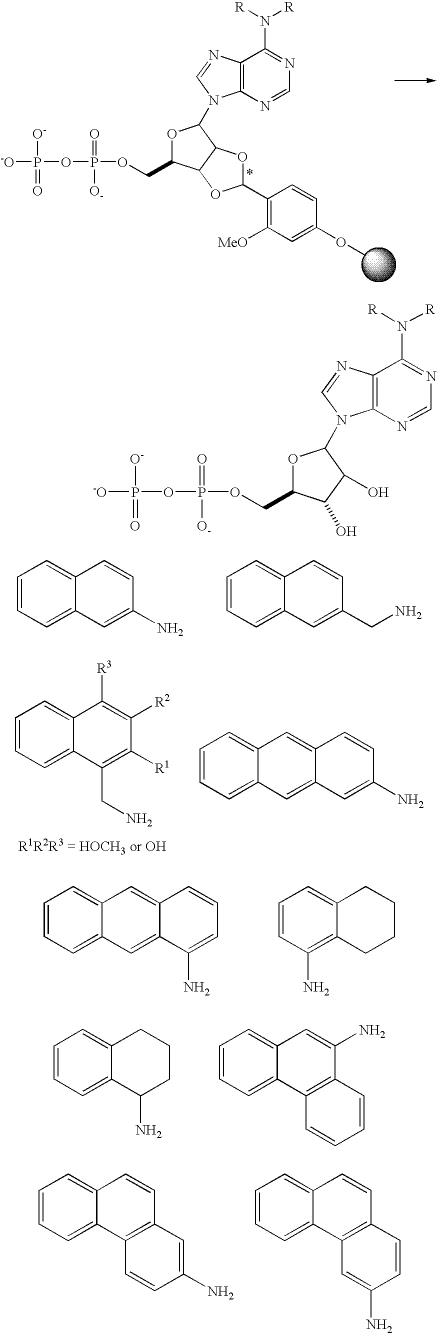

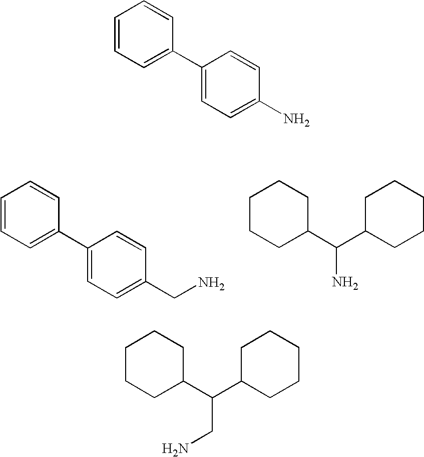

The use of a solid-phase synthesis in preparing RecA inhibitor candidates is attractive for many reasons. A similar approach has been used to synthesize a 25,000-member library as discrete compounds in milligram quantities (Epple et al., 2003). Further, the use of solid-phase synthesis overcomes several yield-limiting problems that can be associated with traditional solution-phase methods, including solubility of the 6-chloropurine riboside and the difficulty of completely removing excess amine after the amination. The route described in Scheme 4 is also attractive in that the MB-CHO resin is conjugated to the ribose as a dialkoxybenzylidene and is more efficiently hydrolyzed that a corresponding Wang benzylidene acetal.

Preliminary tests of the solid-phase route have proven successful. Starting with AGROPORE™ MB-CHO support (highly cross-linked macroreticulate resin; available from Aldrich, Milwaukee, Wis., United States of America), attachment of the riboside was indicated by a color change after staining with anisaldehyde. The presence of Cl⁻ in the eluate after the next reaction with benzylamine was confirmed by precipitation with AgNO₃. Finally, the 5′-O-tosyl-N⁶-benzyladenosine was released in good yield using 10% TFA/H₂O (1:1) in dioxane. Using this route, 5′-O-mesylates have been phosphorylated on-resin to produce 5′-diphosphates, methanediphosphates, and imidodiphosphates. The latter are important diphosphate isosteres that are resistant to hydrolysis and serve only poorly as substrates for phosphorylation. As such, the diphosphate isosteres will be important for testing in permeabilized cells.

III.B.2. 5-Propynyl-Uridine Derivatives

Compounds for inhibiting the ability of RecA to hydrolyze ATP also can include modified pyrimidine nucleosides. In some embodiments, provided is a modified 5-propynyl-deoxyuridine having a structure of Formula (II): 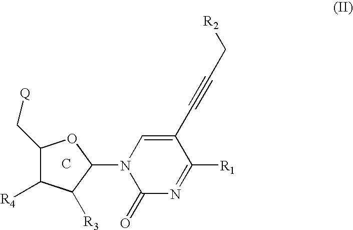
wherein:

R₁ and R₂ are each independently selected from the group consisting of H, alkyl, substituted alkyl, F, Cl, Br, OH, NR₂₂R₂₃ and Y-Z-R₂₄, wherein

R₂₂ and R₂₃ are each independently selected from the group consisting of H, alkyl, substituted alkyl, and alkoxyl;

Y is selected from the group consisting of O, S, NR₂₅ and (CH₂)ₚ, and wherein R₂₅ is selected from the group consisting of H, alkyl, substituted alkyl, and alkoxyl and p is an integer from 1 to 8;

Z can be present or absent and when present is selected from the group consisting of O, S, NR₂₆R₂₇ and C═O, and wherein R₂₆ and R₂₇ are each independently selected from the group consisting of H, alkyl, substituted alkyl, and alkoxyl; and

R₂₄ is selected from the group consisting of H, alkyl, substituted alkyl, alkenyl, substituted alkenyl, alkynyl, substituted alkynyl, aryl and substituted aryl;

R₃ and R₄ are each independently selected from the group consisting of H, F, OH, NR₂₈R₂₉ and R₅—R₆—R₇,

wherein R₂₈ and R₂₉ are each independently selected from the group consisting of H, alkyl, substituted alkyl, and alkoxyl;

R₅ is selected from the group consisting of O and NR₃₀, and wherein R₃₀ is selected from the group consisting of H, alkyl, substituted alkyl, and alkoxyl;

R₆ is selected from the group consisting of (CH₂)_{q}, CF₂ and C═O, and wherein q is an integer from 1 to 8; and

R₇ can be present or absent and when present is selected from the group consisting of H, alkyl, substituted alkyl, alkenyl, substituted alkenyl, alkynyl, substituted alkynyl, aryl and substituted aryl, or

R₃ and R₄ can together with ring C form the following five-membered heterocyclic ring structure:

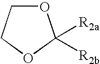

wherein R₂ₐ and R_{2b} are each independently selected from the group consisting of H, alkyl, substituted alkyl, alkenyl, substituted alkenyl, alkynyl, substituted alkynyl, aryl and substituted aryl; and

Q is selected from the group consisting of:

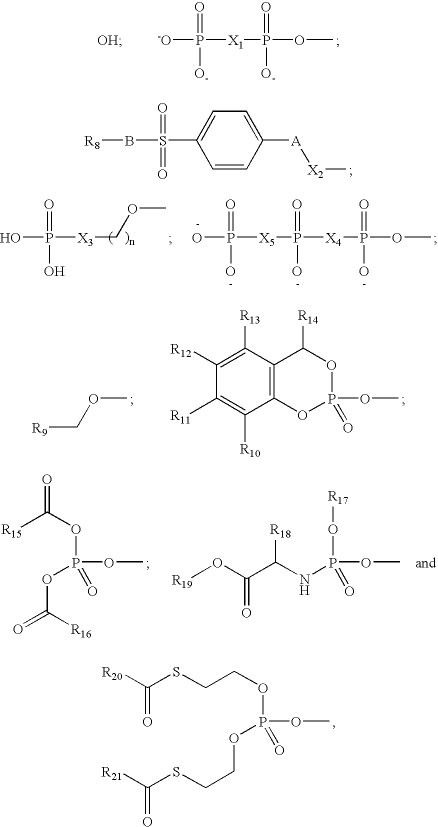

wherein X₁, X₂, X₃, X₄ and X₅ are each independently selected from the group consisting of O, NR₃₁, CH₂ and CF₂, and wherein R₃₁ is selected from the group consisting of H, alkyl, substituted alkyl, and alkoxyl;

A and B are each independently selected from the group consisting of O, NR₃₂, CH₂, CF₂ and C═O, and wherein R₃₂ is selected from the group consisting of H, alkyl, substituted alkyl, and alkoxyl;

n is an integer from 0 to 4;

R₈ is selected from the group consisting of H, alkyl, substituted alkyl, alkenyl, substituted alkenyl, alkynyl, substituted alkynyl, aryl and substituted aryl;

R₉ is selected from the group consisting of 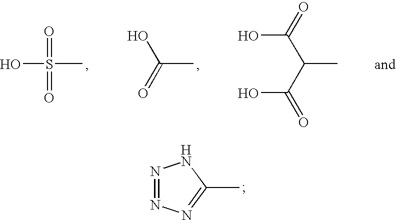
and

R₁₀, R₁₁, R₁₂, R₁₃, R₁₄, R₁₅, R₁₆, R₁₇, R₁₈, R₁₉, R₂₀ and R₂₁, are each independently selected from the group consisting of H, alkyl, substituted alkyl, alkenyl, substituted alkenyl, alkynyl, substituted alkynyl, aryl and substituted aryl;

or a pharmaceutically acceptable salt thereof.

The compounds 5-iodo-2′-deoxyruidine and 5-methyl4-triazolopyrimdin-2-one 2′-deoxyribonucleoside (Singleton et al., 2001) represent interesting and versatile synthons for creating pyrimidine nucleotide libraries. In addition to these modified pyrimidines, 2′-O-modified d(PUTP) derivatives (Metzker et al., 1994) and the structurally related 7-propynyl derivatives of 8-aza-7-deazapurine nucleosides (He & Seela, 2002) and phenylimidazole nucleosides (Kiessling, 1992; Wanq et al., 2004; Seley et al., 2005) can be prepared as potential RecA inhibitors by methods described in the art.

III.B.3. Curcumin Derivatives

Screening of compounds for RecA inhibition has indicated that derivatives of the natural product curcumin have inhibitory acitivity. These curcumin derivatives are shown in Scheme 5, below. For example curcumin derivatives bearing two phenolic groups (X═OH; R═H or alkyl) have I50 values near 40 μM.

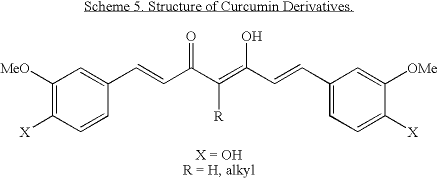

In some embodiments, provided is a curcumin derivative having a structure of Formula (III): 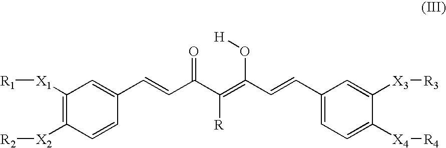
wherein:

X₁, X₂, X₃ and X₄ are each independently selected from the group consisting of H, F, (CH₂)ₙ, CF₂, C═O, O and NR₅, and wherein n is an integer from 1 to 8 and R₅ is selected from the group consisting of H, alkyl, substituted alkyl, and alkoxyl; and

R, R₁, R₂, R₃ and R₄ can each independently be present or absent and if present each is independently selected from the group consisting of H, alkyl, substituted alkyl, alkenyl, substituted alkenyl, alkynyl, substituted alkynyl, aryl and substituted aryl;

or a pharmaceutically acceptable salt thereof.

Additional compounds based upon the structure of curcumin can include a central aromatic or heteroaromatic ring linking the two substituted phenyl rings. Thus, in some embodiments, provided is a compound having a structure of Formula (IV): 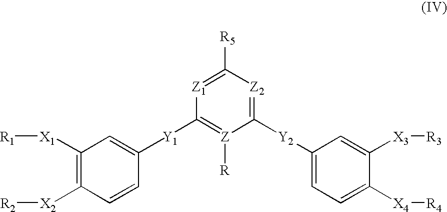
wherein:

X₁, X₂, X₃ and X₄ are each independently selected from the group consisting of H, F, (CH₂)ₙ, CF₂, C═O, O and NR₆, and wherein n is an integer from 1 to 8 and R₆ is selected from the group consisting of H, alkyl, substituted alkyl, and alkoxyl;

Y₁ and Y₂ are each independently selected from the group consisting of C≡C and HC═CH;

Z₁ is selected from the group consisting of CH and N;

Z₂ is selected from the group consisting of CH and N; and

R, R₁, R₂, R₃ R₄ and R₅ can each independently be present or absent and if present each is independently selected from the group consisting of H, alkyl, substituted alkyl, alkenyl, substituted alkenyl, alkynyl, substituted alkynyl, aryl and substituted aryl;

or a pharmaceutically acceptable salt thereof.

III.B.4. Bismuth-Dithiol Complexes

In testing, a 2:1 complex of zinc(II) with 1,4-dithio-L-threitol (L-DTT) inhibited the RecA ATPase activity at >25 μM complex. To determine whether Zn²⁺ or L-DTT can inhibit RecA independently, the inhibitory ability of the two members of the complex was also tested separately. ZnCl₂ by itself exhibited a degree of inhibition similar to the complex while L-DTT alone showed no inhibition of RecA activity. In addition to the inhibition of ATP turnover by Zn²⁺ and Zn·L-DTT, visual inspection revealed that the addition of either free or complexed zinc at concentrations ≧200 μM caused turbidity in the solution. This observation suggested that the apparent inhibition of RecA activity may result from precipitation of RecA from solution, rather than the classical inhibition observed with rho. Indeed, Zn(II), Cu(II), and Hg(II)—in the absence of any dithiol ligand—inactivate the RecA protein in vitro by initiating protein aggregation.

Of particular interest with respect to the development of metal containing inhibitors of RecA is the fact that bismuth compounds have played a prominent role in bioinorganic chemistry and therapy for more than 200 years (Briand & Burford, 1999). Additional testing showed that a 3:1 complex, termed BiBAL, of bismuth(III) with 2,3-dimercapto-1-propanol (British Anti-Lewisite) efficiently inhibits the RecA ATPase activity in vitro. BiBAL (/₅₀=21 μM) is a more efficient inhibitor than Bi(NO₃)₃. Moreover, while Bi³⁺ precipitates RecA in vitro—in a similar fashion to Zn²⁺ and other divalent metals previously tested—BiBAL does not do so. Apparently, the dithiol ligand BAL potentiates the propensity of Bi³⁺ to aggregate RecA. In this context, it is noteworthy that BiBAL appears to inhibit the RecA ATPase activity with a 1:1 stoichiometry (judged by apparent Hill parameters for the dose-response curves), while those metals (and their complexes) that precipitate RecA show much steeper dose-response behavior.

In some embodiments of the presently disclosed subject matter, provided is a compound comprising a bismuth-dithiol complex that comprises bismuth(III) ion and a dithiol compound, wherein the dithiol compound has the general formula (V): 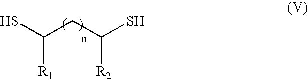
wherein:

n is an integer from 0 to 2; and

R₁ and R₂ are each independently selected from the group consisting of H, alkyl, substituted alkyl, alkenyl, substituted alkenyl, alkynyl, substituted alkynyl, aryl and substituted aryl, or

R₁ and R₂ can together form a five-, six- or seven-membered cycloalkyl, substituted cycloalkyl, heterocyclic, substituted heterocyclic, aryl, or substituted aryl ring structure;

or a pharmaceutically acceptable salt thereof.

Compounds of Formula (V) thus include 1,2-,1,3-, and 1,4-dithiols including commercially available dithiols, as well as novel dithiols prepared according to conventional synthetic organic methods known in the art.

Compounds of formula (V) can be mixed with Bi³⁺ compounds, such as Bi(NO₃)₃, in aqueous solutions to form the bismuth-dithiol complexes. Such solutions can contain other water miscible solvents, such as alcohols and diols. A suitable solution, for example, is an aqueous solution containing 50% 1,2-propane diol.

Additionally, the dithiol compound of the bismuth-dithiol complex can be conjugated (i.e., tethered or covalently bound) to a substituent of an antibiotic. For example, it is believed that the bismuth-dithiol complexes may act synergistically with known antibiotics, such as known genotoxins and replication inhibitors, including mitomycin C (MMC), ciprofloxacin, and norfloxacin. Upon observation of such a synergistic antibiotic effect, a conjugate of the dithiol ligand and the antibiotic can be synthesized. One of general skill in the art can prepare conjugates of both mitomycin (Na et al., 2002; Lee & Kohn, 2004; Lee & Kohn, 2005) and fluoroquinolones (Baker et al., 2004). Broad synthetic strategies for antibiotic thiol ligands are shown in Scheme 6, below.

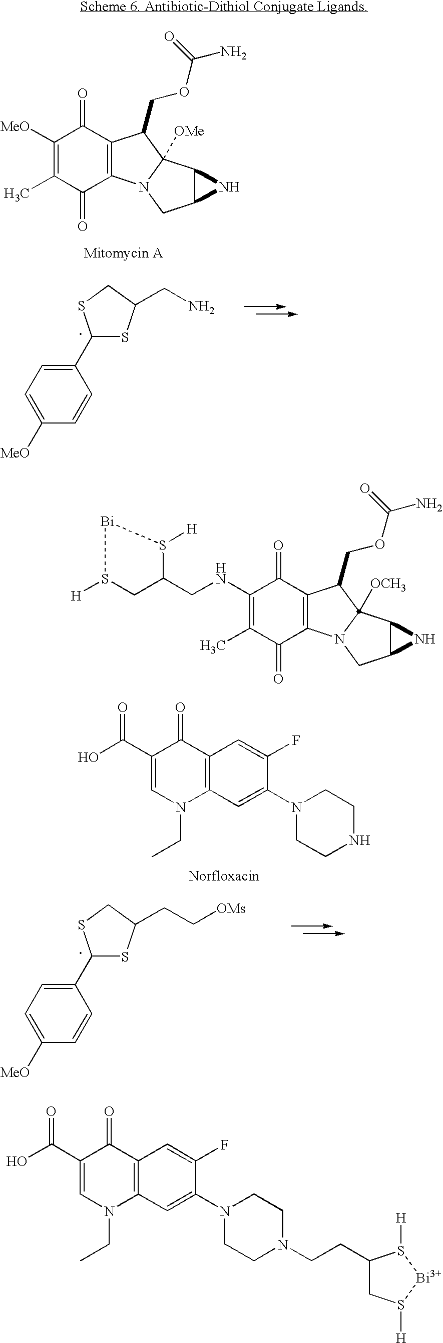

Kohn and coworkers have demonstrated that intramolecular (tethered) thiols can autoactivate MMC derivatives for DNA cross-linking and that this autoactivation is sensitive to the distance between the SH group and the mitomycin core (Na et al., 2002). Bismuth (i.e., Bi³⁺) chelation of a dithiol moiety appended on MMC could serve to protect the core from autoactivation, allowing MMC to reach its chromosomal target where the Bi³⁺ is released for its IRA function and the dithiol-appended MMC is autoactivated for DNA damage.

### IV. PHARMACEUTICAL FORMULATIONS

The term “active compounds” as used herein refers to the RecA activity modifiers described hereinabove. Thus, the term “active compounds” refers to RecA N-terminal mimetics and small molecule compounds that modulate RecA activity, such as nucleoside derivates including N⁶-modified adenosine derivatives (compounds of Formula (I)) and 5-propynyl-uridine derivatives (compounds of Formula (II)), curcumin derivatives (i.e., compounds of Formula (III-IV), and bismuth-dithiol complexes (i.e., complexes of bismuth and compounds of Formula (V)). Pharmaceutical formulations comprising the aforementioned active compounds are provided herein. These pharmaceutical formulations can comprise active compounds as described herein, in a pharmaceutically acceptable carrier. Pharmaceutical formulations can be prepared for oral, intravenous, or aerosol administration as discussed in greater detail below. Also, the presently disclosed subject matter provides such acitve compounds that have been lyophilized and that can be reconstituted to form pharmaceutically acceptable formulations for administration, for example, as by intravenous or intramuscular injection.

Pharmaceutically acceptable salts include those derived from pharmaceutically acceptable inorganic or organic bases and acids. Non-limiting examples of such salts are (a) acid addition salts formed with inorganic acids (for example, hydrochloric acid, hydrobromic acid, sulfuric acid, phosphoric acid, nitric acid, carbonic acid (i.e., carbonate and bicarbonate salts) and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acids, naphthalenedisulfonic acids, and polygalacturonic acid; (b) base addition salts formed with cations such as those derived from alkali metals, those derived from alkaline earth metals, sodium, potassium, zinc, calcium, bismuth, barium, magnesium, aluminum, copper, cobalt, nickel, cadmium, lithium, and the like, or with an organic cation, including ammonium salts. The terms “ammonium salt” or “ammonium salts” are used to describe salts derived from ammonia as well as those derived from alkyl amines, dialkyl amines, trialkyl amines, aralkyl amines, aryl amines, dialkyl-aralkyl amines, etc., and the terms “quaternary ammonium salt” or “quaternary ammonium salts” are used to describe, but are not limited to: tetraalkylammonium salts, trialkylmonobenzylammonium salts, trialkylphenylammonium salts, dialkyldiarylammonium salts, tribenzylalkylammonium salts, trialkylaralkylammonium salts etc. and also salts where the ammonium cationic unit or quaternary ammonium cationic unit is part of, or bound to a polymer chain.

Active compounds including more than one anionic or cationic group that are capable of forming a salt may form salts including combinations of counterions of pharmaceutically acceptable acids or bases. Thus, polyanionic active compounds, such as diphosphates and triphosphates, can form salts with two or more cations. For example, a diphosphate can form a monosodium/monolithium salt.

The therapeutically effective dosage of any specific active compound, the use of which is within the scope of embodiments described herein, will vary somewhat from compound to compound, and patient to patient, and will depend upon the condition of the patient and the route of delivery. As a general proposition, a dosage from about 0.1 to about 50 mg/kg will have therapeutic efficacy, with all weights being calculated based upon the weight of the active compound, including the cases where a salt is employed. Toxicity concerns at the higher level can restrict intravenous dosages to a lower level, such as up to about 10 mg/kg, with all weights being calculated based on the weight of the active base, including the cases where a salt is employed. A dosage from about 10 mg/kg to about 50 mg/kg can be employed for oral administration. Typically, a dosage from about 0.5 mg/kg to 5 mg/kg can be employed for intramuscular injection. Preferred dosages are 1 μmol/kg to 50 μmol/kg, and more preferably 22 μmol/kg and 33 μmol/kg of the compound for intravenous or oral administration. The duration of the treatment is usually once per day for a period of two to three weeks or until the condition is essentially controlled. Lower doses given less frequently can be used prophylactically to prevent or reduce the incidence of recurrence of a disease or to preemptively treat a subject at high risk of developing a disease treatable by inhibiting the immunoproteasome.

In accordance with the presently disclosed methods, pharmaceutically active compounds as described herein can be administered orally as a solid or as a liquid, or can be administered intramuscularly or intravenously as a solution, suspension, or emulsion. Alternatively, the active compounds or salts also can be administered by inhalation, intravenously, or intramuscularly as a liposomal suspension. When administered through inhalation the active compound or salt should be in the form of a plurality of solid particles or droplets having a particle size from about 0.5 to about 5 microns, and preferably from about 1 to about 2 microns.

Pharmaceutical formulations suitable for intravenous or intramuscular injection are further embodiments provided herein. The pharmaceutical formulations comprise an active compound or a pharmaceutically acceptable salt thereof, in any pharmaceutically acceptable carrier. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. The dispensing is preferably done by an aseptic method. Sterilized closures can then be placed on the vials and, if desired, the vial contents can be lyophilized.

In addition to compounds disclosed herein, or their salts, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. The antimicrobial preservative is typically employed when the formulation is placed in a vial designed for multi-dose use. The pharmaceutical formulations described herein can be lyophilized using techniques well known in the art. In some embodiments, the active compounds disclosed herein can be administered in combination with one or more compounds that protect the active compounds from enzymatic degradation, e.g., protease and peptidase inhibitors such as alpha-1 antiprotease, captropril, thiorphan, and the HIV protease inhibitors, and the like.

In yet another embodiment of the subject matter described herein, there is provided an injectable, stable, sterile formulation comprising an active compound, or a salt thereof, in a unit dosage form in a sealed container. The active compound or salt is provided in the form of a lyophilizate, which is capable of being reconstituted with a suitable pharmaceutically acceptable carrier to form a liquid formulation suitable for injection thereof into a subject. The unit dosage form typically comprises from about 10 mg to about 10 grams of the compound salt. When the active compound or salt is substantially water-insoluble, a sufficient amount of emulsifying agent, which is physiologically acceptable, can be employed in sufficient quantity to emulsify the compound or salt in an aqueous carrier. One such useful emulsifying agent is phosphatidyl choline.

Other pharmaceutical formulations can be prepared from the water-insoluble active compounds disclosed herein, or salts thereof, such as aqueous base emulsions. In such an instance, the formulation will contain a sufficient amount of pharmaceutically acceptable emulsifying agent to emulsify the desired amount of the compound or salt thereof. Particularly useful emulsifying agents include phosphatidyl cholines and lecithin.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound will be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques.

The liposomal formulations comprising the active compounds disclosed herein can be lyophilized to produce a lyophilizate, which can be reconstituted with a pharmaceutically acceptable carrier, such as water, to regenerate a liposomal suspension.

Pharmaceutical formulations also are provided which are suitable for administration as an aerosol by inhalation. These formulations comprise a solution or suspension of a desired active compound described herein or a salt thereof, or a plurality of solid particles of the compound or salt. The desired formulation can be placed in a small chamber and nebulized. Nebulization can be accomplished by compressed air or by ultrasonic energy to form a plurality of liquid droplets or solid particles comprising the compounds or salts. The liquid droplets or solid particles should have a particle size in the range of about 0.5 to about 10 microns, more preferably from about 0.5 to about 5 microns. The solid particles can be obtained by processing the solid compound or a salt thereof, in any appropriate manner known in the art, such as by micronization. Most preferably, the size of the solid particles or droplets will be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

When the pharmaceutical formulation suitable for administration as an aerosol is in the form of a liquid, the formulation will comprise a water-soluble active compound in a carrier that comprises water. A surfactant can be present, which lowers the surface tension of the formulation sufficiently to result in the formation of droplets within the desired size range when subjected to nebulization.

As indicated, both water-soluble and water-insoluble active compounds are provided. As used herein, the term “water-soluble” is meant to define any composition that is soluble in water in an amount of about 50 mg/mL, or greater. Also, as used herein, the term “water-insoluble” is meant to define any composition that has a solubility in water of less than about 20 mg/mL. In some embodiments, water-soluble compounds or salts can be desirable whereas in other embodiments water-insoluble compounds or salts likewise can be desirable.

### V. METHODS OF SCREENING FOR COMPOUNDS THAT MODULATE RecA ACTIVITY

The presently disclosed subject matter provides high-throughput screening (HTS) assays for monitoring the modulation of RecA protein activities in order to test large numbers of candidate compounds in rapid fashion and select those compounds capable of modulating RecA protein activity. In one embodiment, the presently disclosed subject matter provides a method for identifying compounds that modulate RecA protein activity comprising contacting a candidate compound with a RecA protein and determining whether the candidate compound modulates the activity of the RecA protein.

The bacterial RecA protein participates in a remarkably diverse set of functions, which are involved in the maintenance of genomic integrity or, paradoxically, the induction of a mutational program. RecA is a central component in both the catalysis of recombinational DNA repair and the regulation of the cellular SOS response. Despite the mechanistic differences of its functions, all require formation of an active RecA-ATP-ssDNA complex, also referred to herein as a nucleoprotein filament (NPF). Because formation of an active RecA-ATP-ssDNA complex (NPF) is an obligate first step for all known RecA functions, inhibition can be achieved either by blocking protein-protein interactions between monomers or by preventing activation of the filament. Filament formation normally results in ATP hydrolysis, which is necessary for controlling SOS induction as well as for the late stages of recombinational DNA repair. Hence, ATP hydrolysis serves as a useful indicator of active NPF formation.

Therefore, the presently disclosed subject matter provides assays developed to characterize the level of inhibition of NPF formation and NPF activation (ATP hydrolysis) by candidate compounds. In one embodiment, an assay is provided which can monitor inhibition of NPF formation. In another embodiment, an assay is provided which can characterize inhibition of ATP hydrolysis.

The assays can be utilized separately, or in combination. Together, the assays can complement one another to allow maximum throughput of candidate compound (e.g., compound library) screening while providing for maximum characterization of first-assay “hits”. In a particular embodiment, the assay for inhibition of NPF formation can provide rapid screening of candidate compounds (e.g., IRAs), and apparent hits can then be further screened (and characterized quantitatively, if desired) using the ATPase hydrolysis assay.

V.A. NPF Formation Assay

In some embodiments, the presently disclosed subject matter provides a method for identifying compounds that modulate RecA protein activity comprising contacting a candidate compound with a RecA protein and measuring the interference of assembly of monomeric RecA protein subunits into a NPF when the candidate compound contacts the RecA protein, to thereby determine whether the candidate compound can modulate RecA activity. In some embodiments, measuring the interference of assembly of monomeric RecA protein subunits into the NPF comprises measuring the amount of monomeric RecA protein subunits released from the nucleoprotein filament.

This assay takes advantage of the observation that RecA dissociates from DNA upon ADP binding (Roca & Singleton, 2003) and allows the rapid screening of potential candidate compounds (e.g., IRAs) for those that prevent active nucleoprotein filament formation. Upon RecA activation by binding ATP, the complex formed between RecA and ssDNA plays a role in directing all RecA functions.

In one exemplary embodiment, the assay is conducted by incubating a candidate compound with RecA and a ssDNA molecule (e.g., (dT)₃₆), and then immobilizing the ssDNA and any bound RecA. This takes advantage of the fact that binding of ADP to RecA reduces the apparent stability and affinity of the RecA-ssDNA complex. In some embodiments, the assay is conducted by pre-incubating the candidate compound with RecA and then adding the binary complex to the immobilized (dT)₃₆.

In some embodiments, the ssDNA molecule is immobilized on streptavidin-paramagnetic particles (SA-PMP). Immobilizing the ssDNA permits determination of whether RecA is released from the nascent nucleoprotein filament upon addition of a putative inhibitor candidate compound by measuring the amount of unbound RecA in the supernatant.

The unbound RecA can be measured using, for example, a Bradford assay, as is generally known in the art. Unbound RecA in the supernatant can also be measured by using RecA protein labeled at its C-terminus by a resorufin-bis-arsenical compound (ReAsH), for example as described by Adams et al. (Adams et al., 2002). RecA-ReAsH can provide sensitive detection of sub-microgram quantities of released RecA. This method provides a high-throughput screen for RecA inhibition by a large variety of candidate compounds (e.g., nucleotide analogs and peptides).

V.B. ATP Hydrolysis Assay

The first step in both RecA-mediated SOS induction and recombinational DNA repair is the binding of RecA to ATP and ssDNA to form an active nucleoprotein filament. NPF formation normally results in ATP hydrolysis, which is necessary for controlling SOS induction as well as for the late stages of recombinational DNA repair. Hence, ATP hydrolysis serves as a useful indicator of active filament formation, and the abrogation of ATPase activity can be an important aspect of RecA inhibition.

Thus, in one embodiment, the presently disclosed subject matter provides a method for identifying compounds that modulate RecA protein activity comprising contacting a candidate compound with a RecA protein and measuring inhibition of ATP hydrolysis by the RecA protein, to thereby determine whether the candidate compound can modulate RecA activity. This assay provides a sensitive, quantitative measure of competitive inhibition of RecA-catalyzed ATP hydrolysis.

In some embodiments, measuring inhibition of ATP hydrolysis by the RecA protein comprises measuring the decrease in production of phosphate resulting from inhibition of the RecA protein ATP hydrolysis by the candidate compound. In other embodiments, measuring inhibition of ATP hydrolysis by the RecA protein comprises measuring the decrease in the production of ADP resulting from inhibition of the RecA protein ATP hydrolysis by the candidate compound.

In one exemplary embodiment of the assay method, enzymic release of free orthophosphate (HₓPO₄^{(3-x)-} or Pᵢ) is measured. A non-radioactive, microplate-based ATPase assay utilizing for example Malachite Green (e.g., BIOMOL GREEN™ reagent (Biomol International., L.P., Plymouth Meeting, Pa., U.S.A.)) can be used.

In another exemplary embodiment, the assay method utilizes an enzyme-dependent Pᵢ release assay that couples the action of purine nucleoside phosphorylase to the fluorogenic conversion of Amplex Red to resorufin, producing a highly sensitive, real-time method for measuring [Pᵢ], even at very low levels.

Both of the exemplary embodiments disclosed for measuring ATP hydrolysis are homogeneous and have no dependence on a nucleotide diphosphate for the detection methods, so either can be utilized as a sensitive method of detecting inhibition of RecA ATPase activity by candidate compounds (e.g., ADP/ATP analogs). However, the resorufin-fluorescence-based assay is continuous and can therefore be used more easily with some applications for determining the inhibition constant for individual nucleotide analogs with RecA. This can permit quick comparison of kinetic parameters for RecA ATPase activity with those that have been determined previously (see e.g., Berger et al., 2001; Brenner et al., 1987) using, for example, an NADH-absorbance based enzyme-coupled assay (Morrical et al., 1986; Harris, 1987; Berger et al., 2001).

### VI. METHODS OF INHIBITING RecA ACTIVITY IN BACTERIA

In some embodiments of the presently disclosed subject matter, a method of inhibiting RecA protein activity in a bacterium is provided. In some embodiments, the method comprises contacting the RecA protein with one or more of the compounds disclosed herein that can modulate RecA activity.

The bacterium of the present method has a functional RecA homolog; that is, the organism has a RecA-like protein that acts to repair damaged DNA and/or mediate SOS-like responses. For example, in some embodiments, the bacterium can be, but is not limited to E. coli, Neisseria sp. (e.g., N. gonorrhoeae, N. meningitidis, etc.), Pseudomonas sp. (e.g., P. aeruginosa, etc.), Mycobacterium sp. (e.g., M. tuberculosis, M. leprae, etc.), Vibrio cholerae, Wisteria monocytogenes, Neisseria sp. (e.g., N. meningitides, N. gonorrhoeae, etc.), Yersinia pestis, Salmonella sp. (e.g., S. typhi, etc.), Shigella sp. (e.g., S. sonnei, S. dysenteriae, etc.), Proteus sp. (e.g., P. vulagris, P. mirabilis), or Bacillus sp. (e.g., B. subtilis, B. anthracis, etc.).

In some embodiments of the method, the compound that contacts the RecA protein is one that can interfere with assembly of monomeric RecA protein subunits into a nucleoprotein filament (NPF). For example, in some embodiments, the compound is a mimetic of the N-terminal helical domain of the RecA protein. A non-limiting exemplary N-terminal helical domain of a RecA protein includes amino acid residues 1-31 of E. coli RecA protein (e.g., SEQ ID NO: 2). However, the presently disclosed subject matter is not intended to be limited only to mimetics of E. coli N-terminal helical domain, but rather is inclusive of RecA N-terminal helical domains from other bacteria as well. For example, Yersinia pestis, Salmonella typhi, Shigella sonnei, and Proteus vulgaris each comprise RecA proteins having an N-terminal helical domain identical to that of E. coli. Numerous other bacteria known in the art express RecA proteins having N-terminal domains homologous to the sequences disclosed herein and are therefore intended as well to be encompassed by the presently disclosed subject matter. As such, in some embodiments, the N-terminal helical domain mimetic compound comprises the amino acid sequence B—X₃-Z-X₂-Z-Z-X₂-Z-X₃-Z (SEQ ID NO:3), wherein B is lysine or arginine; Xₙ is n number of any amino acids and X can be the same or different amino acids; and Z is alanine, valine, leucine, isoleucine, phenylalanine, or methionine.

In some embodiments of the method, the compound that contacts the RecA protein is one that can interfere with ATP hydrolysis by the RecA protein. In some embodiments, the compound is selected from the group consisting of a modified adenosine, a modified 5-propynyl-deoxyuridine, a curcumin derivative and a bismuth-dithiol complex. In some embodiments, the compound is a modified adenosine selected from the group consisting of a modified adenosine monophosphate, a modified adenosine diphosphate and a modified adenosine triphosphate. Further, in some embodiments, the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate. In some embodiments, the compound is a compound of Formula (I-V) disclosed herein.

### VII. METHODS OF TREATING A BACTERIAL INFECTION IN A SUBJECT

In some embodiments of the presently disclosed subject matter, a method of treating a bacterial infection in a subject is provided. In some embodiments, the method comprises administering to the subject a therapeutically effective amount of a pharmaceutical composition comprising a compound that modulates bacterial RecA protein activity.

The bacterial infection in the subject can be caused by a bacterium having a functional RecA homolog; that is, the organism has a RecA-like protein that acts to repair damaged DNA and/or mediate SOS-like responses in the bacterium. For example, in some embodiments, the bacterium causing the bacterial infection can be, but is not limited to E. Coli, Neisseria sp. (e.g., N. gonorrhoeae, N. meningitidis, etc.), Pseudomonas sp. (e.g., P. aeruginosa etc.), Mycobacterium sp. (e.g., M. tuberculosis, M. leprae, etc.), Vibrio cholerae, Listeria monocytogenes, Neisseria sp. (e.g., N. meningitides, N. gonorrhoeae, etc.), Yersinia pestis, Salmonella sp. (e.g., S. typhi, etc.), Shigella sp. (e.g., S. sonnei, S. dysenteriae, etc.), Proteus sp. (e.g., P. vulagris, P. mirabilis), or Bacillus sp. (e.g., B. subtilis, B. anthracis, etc.).

As used herein, the terms “therapeutically effective amount” and “effective amount” are used interchangeably and mean a dosage sufficient to provide treatment for the disease state being treated (e.g., the bacterial infection). This can vary depending on the subject, the disease and the treatment being effected.

In some embodiments of the treatment method, the compound that modulates bacterial RecA protein activity is one that can interfere with assembly of monomeric RecA protein subunits into a nucleoprotein filament (NPF). For example, in some embodiments, the compound is a mimetic of the N-terminal helical domain of the RecA protein. A non-limiting exemplary N-terminal helical domain of a RecA protein includes amino acid residues 1-31 of E. coli RecA protein (e.g., SEQ ID NO:2). However, the presently disclosed subject matter is not intended to be limited only to mimetics of E. coli N-terminal helical domain, but rather is inclusive of RecA N-terminal helical domains from other bacteria as well.

For example, Yersinia pestis, Salmonella typhi, Shigella sonnei, and Proteus vulgaris each comprise RecA proteins having an N-terminal helical domain identical to that of E. coli. Numerous other bacteria known in the art express RecA proteins having N-terminal domains homologous to the sequences disclosed herein and are therefore intended as well to be encompassed by the presently disclosed subject matter. As such, in some embodiments, the N-terminal helical domain mimetic compound comprises the amino acid sequence B—X₃-Z-X₂-Z-Z-X₂-Z-X₃-Z (SEQ ID NO:3), wherein B is lysine or arginine; Xₙ is n number of any amino acids and X can be the same or different amino acids; and Z is alanine, valine, leucine, isoleucine, phenylalanine, or methionine.

In some embodiments of the treatment method, the compound that contacts the RecA protein is one that can interfere with ATP hydrolysis by the RecA protein. In some embodiments, the compound is selected from the group consisting of a modified adenosine, a modified 5-propynyl-deoxyuridine, a curcumin derivative and a bismuth-dithiol complex. In some embodiments, the compound is a modified adenosine selected from the group consisting of a modified adenosine monophosphate, a modified adenosine diphosphate and a modified adenosine triphosphate. Further, in some embodiments, the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate. In some embodiments, the compound is a compound of Formula (I-V) disclosed herein.

The pharmaceutical composition comprising the compound can be formulated as disclosed in detail in Section IV herein. In some embodiments, the pharmaceutical composition further comprises an antibiotic. In some embodiments, the antibiotic is a replication inhibitor. Further, in some embodiments, the replication inhibitor can be selected from the group consisting of actinomycins, adriamycin, aflatoxins, altromycins, anthramycin, bleomycins, calicheamicins, carmustine (BCNU), daunomycin, distamycins, dynemicins, echinomycin, esperamicins, kericidin, mitomycins, neocarzinostatin, netropsins, nitric oxide, nitrogen mustards, nitrosamines, peroxides, pluramycins, pyrrolo[1,4]benzodiazepines, sibiromycin, streptozotocin, tomamycin, beta-lactams, quinolones, fluoroquinolones, DNA Gyrase inhibitors, DNA Polymerase I inhibitors, nucleoside and nucleotide analogs, ribonucleotide reductase inhibitors, antifolates, and DNA biosynthesis inhibitors. In some specific embodiments, the antibiotic can be amoxicillin, ciprofloxacin, clindamycin, doxycycline, erythromycin, or naldixic acid.

Further with respect to the treatment methods of the presently disclosed subject matter, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

Suitable methods for administering to a subject a pharmaceutical composition comprising a compound that modulates bacterial RecA protein activity in accordance with the methods of the present subject matter include but are not limited to systemic administration, parenteral administration (including intravascular, intramuscular, intraarterial administration), oral delivery, buccal delivery, subcutaneous administration, inhalation, intratracheal installation, surgical implantation, transdermal delivery, local injection, and hyper-velocity injection/bombardment. Where applicable, continuous infusion can enhance drug accumulation at a target site (see, e.g., U.S. Pat. No. 6,180,082).

The particular mode of pharmaceutical composition administration used in accordance with the methods of the presently disclosed subject matter depends on various factors, including but not limited to the carrier employed, the severity of the bacterial infection to be treated, and mechanisms for metabolism or removal of the pharmaceutical composition following administration.

### VIII. METHODS OF IMPEDING ANTIBIOTIC RESISTANCE BY A BACTERIUM

In some embodiments of the presently disclosed subject matter, a method for impeding development of resistance to an antibiotic by a bacterium is provided. In some embodiments, the method comprises contacting the bacterium with a composition comprising an antibiotic and a compound that modulates activity of a RecA protein of the bacterium.

A compound capable of impeding development of antibiotic resistance in a bacterium is one in which a decrease in antibiotic resistance, or no increase in antibiotic resistance when expected in the bacterium is measured, as compared to a similar bacterium under comparable circumstances in the absence of the compound. A variety of techniques generally known in the art can be used to measure antibiotic resistance. For example, growth assays can be performed with bacteria of interest (e.g., pathogenic bacteria), or correlative organisms (e.g., E. coli) in the presence of different antibiotics (e.g., amoxicillin, ciprofloxacin, clindamycin, doxycycline, erythromycin, and naldixic acid) and varying concentrations of antibiotics. The development of resistance can be measured as a change in turbidity (e.g., at OD₅₉₀) after a selected incubation time, depending on the growth rate of the particular bacteria in growth media. These data then are used as the standard control for the particular bacteria in combination with the particular antibiotic under the particular growing conditions. To evaluate the potential of candidate compounds to impede the development of resistance, comparable growth assays can be performed in the presence of non-growth inhibitory concentrations of the candidate compound. An increase in the time required to develop turbidity in the presence of the candidate compound can be interpreted as the compound impeding the development of antibiotic resistance.

The bacterium of the present methods has a functional RecA homolog; that is, the organism has a RecA-like protein that acts to repair damaged DNA and/or mediate SOS-like responses. For example, in some embodiments, the bacterium can be, but is not limited to E. coli, Neisseria sp. (e.g., N. gonorrhoeae, N. meningitidis, etc.), Pseudomonas sp. (e.g., P. aeruginosa etc.), Mycobactenum sp. (e.g., M. tuberculosis, M. leprae, etc.), Vibrio cholerae, Listena monocytogenes, Neisseria sp. (e.g., N. meningitides, N. gonorrhoeae, etc.), Yersinia pestis, Salmonella sp. (e.g., S. typhi, etc.), Shigella sp. (e.g., S. sonnei, S. dysenteriae, etc.), Proteus sp. (e.g., P. vulagris, P. mirabilis), or Bacillus sp. (e.g., B. subtilis, B. anthracis, etc.).

In some embodiments of the method, the compound that contacts the RecA protein is one that can interfere with assembly of monomeric RecA protein subunits into a nucleoprotein filament (NPF). For example, in some embodiments, the compound is a mimetic of the N-terminal helical domain of the RecA protein. A non-limiting exemplary N-terminal helical domain of a RecA protein includes amino acid residues 1-31 of E. coli RecA protein (e.g., SEQ ID NO:2). However, the presently disclosed subject matter is not intended to be limited only to mimetics of E. coli N-terminal helical domain, but rather is inclusive of RecA N-terminal helical domains from other bacteria as well. For example, Yersinia pestis, Salmonella typhi, Shigella sonnei, and Proteus vulgaris each comprise RecA proteins having an N-terminal helical domain identical to that of E. coli. Numerous other bacteria known in the art express RecA proteins having N-terminal domains homologous to the sequences disclosed herein and are therefore intended as well to be encompassed by the presently disclosed subject matter. As such, in some embodiments, the N-terminal helical domain mimetic compound comprises the amino acid sequence B—X₃-Z-X₂-Z-Z-X₂-Z-X₃-Z (SEQ ID NO:3), wherein B is lysine or arginine; Xₙ is n number of any amino acids and X can be the same or different amino acids; and Z is alanine, valine, leucine, isoleucine, phenylalanine, or methionine.

In some embodiments of the method, the compound that contacts the RecA protein is one that can interfere with ATP hydrolysis by the RecA protein. In some embodiments, the compound is selected from the group consisting of a modified adenosine, a modified 5-propynyl-deoxyuridine, a curcumin derivative and a bismuth-dithiol complex. In some embodiments, the compound is a modified adenosine selected from the group consisting of a modified adenosine monophosphate, a modified adenosine diphosphate and a modified adenosine triphosphate. Further, in some embodiments, the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate. In some embodiments, the compound is a compound of Formula (I-V) disclosed herein.

In some embodiments, the antibiotic is a replication inhibitor. Further, in some embodiments, the replication inhibitor can be selected from the group consisting of actinomycins, adriamycin, aflatoxins, altromycins, anthramycin, bleomycins, calicheamicins, carmustine (BCNU), daunomycin, distamycins, dynemicins, echinomycin, esperamicins, kericidin, mitomycins, neocarzinostatin, netropsins, nitric oxide, nitrogen mustards, nitrosamines, peroxides, pluramycins, pyrrolo[1,4]benzodiazepines, sibiromycin, streptozotocin, tomamycin, beta-lactams, quinolones, fluoroquinolones, DNA Gyrase inhibitors, DNA Polymerase I inhibitors, nucleoside and nucleotide analogs, ribonucleotide reductase inhibitors, antifolates, and DNA biosynthesis inhibitors. In some specific embodiments, the antibiotic can be amoxicillin, ciprofloxacin, clindamycin, doxycycline, erythromycin, or naldixic acid.

### IX. METHODS FOR ENHANCING ANTIMICROBIAL ACTIVITY OF AN ANTIBIOTIC

In some embodiments of the presently disclosed subject matter, a method of enhancing antimicrobial activity of an antibiotic is provided. In some embodiments, the method comprises contacting a bacterium with the antibiotic and a compound that modulates activity of a RecA protein of the bacterium.

Enhancement in antimicrobial activity of an antibiotic can be measured by any of several known methods in the art for determining antibiotic susceptibility of bacteria. For example, the minimum inhibitory concentration (MIC) of an antibiotic of interest can be measured in the absence and presence of a candidate compound. A measured decrease in the MIC in the presence of the compound is indicative of enhanced antimicrobial activity from the antibiotic in the presence of the compound.

The bacterium of the present method has a functional RecA homolog; that is, the organism has a RecA-like protein that acts to repair damaged DNA and/or mediate SOS-like responses. For example, in some embodiments, the bacterium can be, but is not limited to E. coli, Neisseria sp. (e.g., N. gonorrhoeae, N. meningitidis, etc.), Pseudomonas sp. (e.g., P. aeruginosa etc.), Mycobacterium sp. (e.g., M. tuberculosis, M. Ieprae, etc.), Vibrio cholerae, Listeria monocytogenes, Neisseria sp. (e.g., N. meningitides, N. gonorrhoeae, etc.), Yersinia pestis, Salmonella sp. (e.g., S. typhi, etc.), Shigella sp. (e.g., S. sonnei, S. dysenteriae, etc.), Proteus sp. (e.g., P. vulagris, P. mirabilis), or Bacillus sp. (e.g., B. subtilis, B. anthracis, etc.).

In some embodiments of the method, the compound that contacts the RecA protein is one that can interfere with assembly of monomeric RecA protein subunits into a nucleoprotein filament (NPF). For example, in some embodiments, the compound is a mimetic of the N-terminal helical domain of the RecA protein. A non-limiting exemplary N-terminal helical domain of a RecA protein includes amino acid residues 1-31 of E. coli RecA protein (e.g., SEQ ID NO:2). However, the presently disclosed subject matter is not intended to be limited only to mimetics of E. coli N-terminal helical domain, but rather is inclusive of RecA N-terminal helical domains from other bacteria as well. For example, Yersinia pestis, Salmonella typhi, Shigella sonnei, and Proteus vulgaris each comprise RecA proteins having an N-terminal helical domain identical to that of E. coli. Numerous other bacteria known in the art express RecA proteins having N-terminal domains homologous to the sequences disclosed herein and are therefore intended as well to be encompassed by the presently disclosed subject matter. As such, in some embodiments, the N-terminal helical domain mimetic compound comprises the amino acid sequence B—X₃-Z-X₂-Z-Z-X₂-Z-X₃-Z (SEQ ID NO:3), wherein B is lysine or arginine; Xₙ is n number of any amino acids and X can be the same or different amino acids; and Z is alanine, valine, leucine, isoleucine, phenylalanine, or methionine.

In some embodiments of the method, the compound that contacts the RecA protein is one that can interfere with ATP hydrolysis by the RecA protein. In some embodiments, the compound is selected from the group consisting of a modified adenosine, a modified 5-propynyl-deoxyuridine, a curcumin derivative and a bismuth-dithiol complex. In some embodiments, the compound is a modified adenosine selected from the group consisting of a modified adenosine monophosphate, a modified adenosine diphosphate and a modified adenosine triphosphate. Further, in some embodiments, the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate. In some embodiments, the compound is a compound of Formula (I-V) disclosed herein.

In some embodiments, the antibiotic is a replication inhibitor. Further, in some embodiments, the replication inhibitor can be selected from the group consisting of actinomycins, adriamycin, aflatoxins, altromycins, anthramycin, bleomycins, calicheamicins, carmustine (BCNU), daunomycin, distamycins, dynemicins, echinomycin, esperamicins, kericidin, mitomycins, neocarzinostatin, netropsins, nitric oxide, nitrogen mustards, nitrosamines, peroxides, pluramycins, pyrrolo[1,4]benzodiazepines, sibiromycin, streptozotocin, tomamycin, beta-lactams, quinolones, fluoroquinolones, DNA Gyrase inhibitors, DNA Polymerase I inhibitors, nucleoside and nucleotide analogs, ribonucleotide reductase inhibitors, antifolates, and DNA biosynthesis inhibitors. In some specific embodiments, the antibiotic can be amoxicillin, ciprofloxacin, clindamycin, doxycycline, erythromycin, or naldixic acid.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently claimed subject matter.

### Materials and Methods for Examples

Reagents and starting materials for the compounds synthesized as described herein were from commercially available sources, such as Aldrich (Milwaukee, Wis., United States of America) and Sigma (St. Louis, Mo., United States of America). Authentic samples of N⁶-(benzyl)-ADP and N⁶-(2-phenethyl)-ADP were purchased from Axxora, LLC (San Diego, Calif., United States of America).

Water content in commercially supplied solvent was typically less than 30 ppm. Doubly distilled, deionized water was used for all aqueous reagents and reactions. Glassware for all reactions was oven-dried at 120° C. overnight and allowed to cool to room temperature in a dessicator. All reactions were conducted under an atmosphere of dry argon or nitrogen. TLC analyses were performed on Whatman K6F silica plates (0.25 mm layer thickness). Flash column chromatography was performed using 230400 mesh silica gel. ¹H NMR (300 MHz) spectra were recorded on a Varian Gemini 2000 spectrometer, and ¹³C (125 MHz) and ³¹P (202.5 MHz) NMR spectra were recorded on a Varian Inova NMR spectrometer. Chemical shifts are reported as 8 values (ppm) downfield relative to TMS (δ 0.0) as an internal reference for ¹H and ¹³C NMR spectra or H₃PO₄ (δ 0.0) as an external reference for ³¹P NMR spectra. The following abbreviations are used for spin multiplicity: s=singlet, d=doublet, t=triplet, and m=multiplet. Low- and high-resolution fast atom bombardment mass spectrometric analyses were performed by the Duke University Center for Mass Spectrometry (Durham, N.C., United States of America) using a Jeol SX-102 instrument.

### Example 1

### Solution-Phase Synthesis of N⁶-Substituted Adenosine Compounds

N⁶-substitution of adenosine was carried out essentially as described previously (Kikugawa et al., 1973). Subsequent phosphorylation of the N⁶-substituted adenosine derivatives was carried out using a procedure modified from the literature (Désaubry et al., 1996).

N⁶-(1-Naphthyl)-Adenosine. To a stirred suspension of 6-chloropurine riboside (200 mg, 0.7 mmol) in 15 mL anhydrous ethanol was added 1-aminonaphthalene (601 mg, 4.2 mmol), and the mixture was brought to reflux and stirred for 5 hours (h). The resulting solution was allowed to cool to room temperature (RT) and then allowed to stand overnight at 4° C. The crystals were isolated by vacuum filtration to afford 4 (164 mg, 59%). Analytical data (¹H NMR in CDCl₃, 300 MHz) were identical to those reported in the literature.

N⁶-(1-Naphthyl)-Adenosine-5′-O-Diphosphate (1). Compound 4 (79 mg, 0.2 mmol) was suspended in 1 mL trimethyl phosphate, and the mixture was cooled to 0° C. To the suspension was added phosphorous oxychloride (40 μL, 0.42 mmol), and the mixture was stirred at 4° C. for 3 h. The resulting homogeneous solution was cooled to 0° C., and tris(tetrabutylammonium) pryrophosphate (1 mmol) in 2 mL anhydrous DMF was added, followed by tributylamine (200 μL, 0.84 mmol). The solution was stirred for 1 min at 0° C. and immediately quenched by the addition of 10 mL aqueous triethylammonium bicarbonate buffer (0.2 M, pH 7.5). The quenched reaction mixture was stirred for ca. 5 min at 0° C. until the visible evolution of gas ceased. The solvent was then evaporated and the residue was purified by DEAE-cellulose column chromatography using a gradient of 0.1 M to 0.3 M aqueous triethylammonium bicarbonate buffer (pH 7.5). The UV-absorbing fractions were pooled into separate samples for further analysis and lyophilized. The resultant syrup was subjected to salt exchange by diluting it with H₂O, loading the sample onto a column of Dowex 50WX8-200, Na⁺ form, and eluting 1 with H₂O. The eluted sample containing the sodium salt of the nucleoside diphosphate was lyophilized to yield 15 mg of compound 1 (Na⁺ salt) (13% yield). Characterization by analytical ion-exchange HPLC (25 cm×4.6 mm Supelcosil SAX1 column, 1 mL/min flow rate) was carried out using a gradient of 5 mM aqueous ammonium phosphate (pH 2.9) to 750 mM aqueous ammonium phosphate (pH 3.8) over 32 min. The HPLC analysis demonstrated a unique retention time relative to the other pooled fractions and a purity ≧95% relative to other UV-absorbing materials.

¹H NMR (D₂O): δ (ppm) 8.59 (s, 1H), 8.10 (s, 1H), 8.00 (m, 1H), 7.95 (m, 2H), 7.60 (m, 3H), 7.52 (m, 1H), 6.10 (d, J=6.0 Hz, 1H), 4.85 (m, 1 H), 4.59 (m, 1H), 4.40 (m, 1H), 4.20 (m, 2H). ¹³C NMR (D₂O/DMSO-d₆): δ (ppm) 155.9, 154.2, 150.9, 141.8, 136.0, 134.5, 131.3, 130.3, 130.3, 129.4, 128.6, 128.4, 127.8, 126.1, 124.4, 121.0, 88.5, 85.9, 76.2, 72.2. ³¹P NMR (D₂O): δ (ppm)−6.9 (d, J=20.6 Hz, 1P), −7.6 (d, J=20.6 Hz, 1P). MS (negative-ion FAB): 552 [M²⁻+H⁺]⁻. High-resolution MS (negative-ion FAB) calcd for C₂₀H₂₀N₅O₁₀P₂ [M²⁻+H⁺]⁻, 552.0685; found, 552.0692.

N⁶-(Benzyl)-Adenosine-5′-O-Diphosphate (2). N⁶-(Benzyl)-Adenosine was prepared analogously to the method described for N⁶-(napthyl)-adenosine in 89 % yield. Compound 2 was prepared from N⁶-(benzyl)-adenosine as described above for 1. Analytical data (¹H NMR in D₂O, 300 MHz) were identical to those reported in the literature as well as for an authentic commercial sample.

N⁶-(2-Phenethyl)-Adenosine-5′-O-Diphosphate (3). N⁶-(2-Phenethyl)-Adenosine was prepared analogously to the synthesis of N⁶-(Napthyl)-Adenosine in 82% yield. Compound 3 was prepared from N⁶-(2-phenethyl)-adenosine as described above for 1. Analytical data (¹H NMR in D₂O, 300 MHz) were identical to those reported in the literature as well as for an authentic commercial sample.

Alternative Procedures for Deionization and Salt Exchange of Compound 1. In order to ensure that the observed inhibitory effects of 1 on the activities of RecA did not result simply from excess salt or other impurities, the final purification of 1 was effected using two alternate procedures. (a) In the first case, purification was carried out as described above until the point of salt exchange. At this point, the aqueous solution was fractionated by preparative RP-HPLC (25 cm×10 mm Beckman Ultrasphere ODS column, 3.0 mL/min flow rate) using a gradient of 0→30% aq CH₃CN over 30 min. The 1-containing fractions were pooled and lyophilized to a dry powder. (b) In the second case, purification was again carried out as described above until the point of salt exchange. At this point, the aqueous solution was fractionated using a 2-mL column of boronic acid resin (BAR). Briefly, the sample was diluted to 10 mL with aqueous buffer containing 50 mM HEPES (pH 8.4) and 50 mM Mg(Oac)₂. The diluted sample was added to BAR, the mixture was gently vortexed and the BAR collected by centrifugation (3000 rpm for 3 min). The BAR was washed two more times with 10 mL of HEPES/Mg buffer. The BAR was gently suspended in 10 mL of 25 mM NH₄HCO₃ (pH 5.5) and transferred to a 1.2-cm (OD) column. The 1-containing fractions were eluted in 2×10 mL NH₄HCO₃ and lyophilized to a dry powder. All analytical and biochemical assay data for samples of 1 prepared in the three different ways were indistinguishable.

### Example 2

### Procedure of the In Vitro Inhibition of NPF Formationa Assay

Doubly distilled, deionized water was used for all aqueous reagents and reactions. ATP, ADP, and ATPγS were from Roche (Nutley, N.J., United States of America). Tris base and DTT were from Fisher Scientific International., Inc. (Hampton, N.H., United States of America). Sodium chloride, magnesium acetate, glycerol, phosphoenolpyruvate, pyruvate kinase, lactic dehydrogenase, and NADH were from Sigma (St. Louis, Mo., United States of America). Biotin-dT₃₆, an oligo(dT)₃₆ with a biotin-TEG at the 3′ end, was purchased from Sigma-Genosys (The Woodlands, Tex., United States of America). Poly(dT) ssDNA (average length=319 bases) was purchased from Amersham Biosciences (Piscataway, N.J., United States of America). Streptavidin Paramagnetic Particles were from Promega Biosciences, Inc. (San Luis Obispo, Calif.) and 96-well microplates (flat- and round-bottom wells) were purchased from Evergreen Scientific (Los Angeles, Calif., United States of America). N⁶-Benzyl-adenosine-5′-O-diphosphate (2) and N⁶-(2-phenethyl)-adenosine-5′-O-diphosphate (3) were obtained from AXXORA (San Diego, Calif., United States of America). RecA protein was purified as previously described (Singleton et al., 2002).

The displacement of RecA from ssDNA in the presence of different nucleotide analogs was monitored using a microplate-based assay. Assays were conducted at 37° C. in aqueous Assay Buffer containing the following final concentrations: 25 mM Tris·HOAc, pH 7.1, 60 mM NaCl, 10 mM Mg(Oac)₂, 2 μM ATPγS, 1 mM DTT, and 5% (v/v) glycerol. The final assay concentrations of RecA protein, biotin-dT₃₆, and nucleotide analog were 4 μM, 18 μM-nts, and 100 μM, respectively. To each well of a round-bottom 96-well microplate was added 10 μL of ATPγS (10 μM), 10 μL NaCl (300 mM), and 30 μL of a premixed solution containing RecA protein (6.4 μM), biotin-dT₃₆ (28.8 μM-nts), nucleotide inhibitor (160 μM), and Assay Buffer (1.6× final concentrations). The microplate was allowed to stand at 37° C. while a second microplate containing washed streptavidin-paramagnetic particles (SA-PMP) was prepared. A 50-μL aliquot of SA-PMP (Promega), suspended at 1 mg/mL, was added to each well of a round-bottom 96-well microplate, the beads were pelleted using a magnet, and the supernatant was removed. The SA-PMP were subsequently washed three times with 50-μL portions of Assay Buffer. After the final wash, the SA-PMP were pelleted, and the supernatant was removed. To each well containing washed SA-PMP was added the appropriate 50-μL solution containing RecA, DNA, nucleotide and Assay Buffer. The SA-PMP were resuspended by gentle pipeting and aspiration, the mixtures were allowed to stand at 37° C. for 15 min, the SA-PMP were pelleted using a magnet, and a 10-μL sample of each supernatant was transferred to a flat-bottom 96-well microplate for analysis of protein content. Bradford Assay analysis was performed using BIORAD® Protein Assay Solution (Bio-Rad Laboratories, Hercules, Calif., United States of America) according the supplier's protocol. After a 5-min reaction at room temperature, the absorbance at 595 nm was measured using a PerkinElmer HTS7000+BioAssay Reader (PerkinElmer, Wellesley, Mass., United States of America) using a 595±35 nm absorbance filter. Absorbance values were converted to protein concentrations by comparison with a standard RecA curve recorded at the same time. Values reported for each nucleotide analog are the mean f one standard deviation for at least three independent measurements.

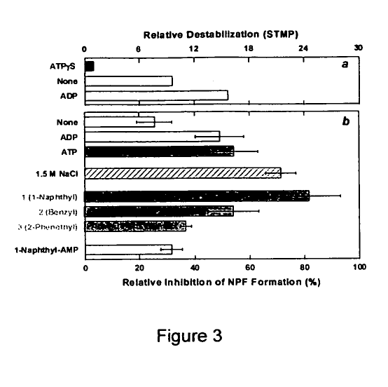

Using this assay, the 16 canonical nucleoside di- and triphosphates—both ribo- and 2′-deoxyribonucleotides—were screened for RecA inhibition (FIG. 3). The results recapitulated what is known about the ability of various nucleotides to bind and activate (or deactivate) the RecA nucleoprotein filament. Indeed, the assay results (FIG. 3b) demonstrate the expected trend in the relative NPF stabilities: RecA·ADP<RecA-only. Moreover, ATP has the same apparent effect on NPF stability as does ADP, consistent with the hydrolysis of ATP to ADP during the experiment. The fact that the amount of RecA protein released in the presence or absence of ADP/ATP is significantly less than that released by the addition of NaCl (final concentration 1.5 M) verifies that substantial protein is coating the immobilized (dT)₃₆. The results for the nucleotides that are known to be RecA substrates were qualitatively similar to those for ADP/ATP (Bisognano et al., 2004).

Further investigation of ADP was conducted using this displacement assay in the form of a titration experiment, and the relative amounts of RecA in the supernatant were plotted as a function of [ADP] and fit to yield an apparent dissociation constant for ADP, K_{d}=84±27 μM, that compares favorably to the values reported in the art. Importantly, this also demonstrates that the assay signal correlates with fraction bound (and K_{d}), suggesting that the assay can be used for semi-quantitative comparison of candidate compounds (e.g., IRAs).

### Example 3

### Procedure for SSDNA-Dependent RecA-Catalyzed ATP-Hydrolysis Assay

The inhibition of RecA ATPase activity by N⁶-(1-napthyl)-ADP (1) was measured using an enzyme-couples assay system essentially as described previously (Berger et al., 2001) with modifications for use in a 96-well plate format. Assays were conducted at 37° C. in 100 μL of aqueous buffer containing 0.2 μM RecA protein, 2 μM-nts poly(dT), 2.3 mM phosphoenolpyruvate, 5 U/mL pyruvate kinase, 5 U/mL lactic dehydrogenase, 2 mM NADH, 25 mM Tris·HOAc, pH 7.1, 10 mM Mg(Oac)₂, 1 mM DTT, 5% (v/v) glycerol and various concentrations of ATP and 1. ATP was serially diluted to yield various concentrations at 10× final concentration in 100 mM Mg(Oac)₂. These solutions served as the start solution for the assay reactions. Likewise, compound 1 was serially diluted in aqueous buffer (25 mM Tris·HOAc, pH 7.1, 5% (v/v) glycerol) to yield a range of solutions containing 5× final concentration. To each well of a flat-bottom 96-well microplate were added 10 μL of the appropriate ATP/Mg(Oac)₂ solution and 20 μL of the appropriate solution of 1. The remaining assay components were combined in a total volume of 70 μL and added to each well containing ATP, Mg(Oac)₂, and 1. The 380-nm absorbance of each solution was recorded every 60 s using a PerkinElmer HTS7000+BioAssay Reader equipped with a 380±10 nm bandpass filter (Andover Corporation, Salem, N.H. United States of America). The reaction velocity (μM min⁻¹) for each unique pair of [ATP] and [1] was calculated from the change in absorbance as a function of time (dA/dt) using the following equation, where 5.47×10⁻⁴ μM⁻¹ is the change in extinction coefficient measured in the microplate reader:

$$V = - \frac{ⅆ A / ⅆ t}{5.47 \times {10}^{- 4} μ {M}^{- 1}} \qquad ( 1 )$$

Each set of data, corresponding to a range of [ATP] concentrations at a single [1], was analyzed using a Michaelis-Menten equation modified for substrate cooperativity (Menge & Bryant, 1988):

$$V = {{k}_{cat} [ RecA ]}_{tot} \cdot \frac{{[ ATP ]}^{3}}{{[ ATP ]}^{3} + {S}_{0.5}^{3}} \qquad ( 2 )$$

where S_{0.5} is the [ATP] when the velocity is half of its maximum value. The K_{ic} for 1 with respect to ATP was determined from a linear fit of a plot of S_{0.5}^{obs} versus [1].

This assay system was also utilized to measure the Michaelis constant for RecA and ATP: S_{0.5}^{ATP}=47 μM with a Hill coefficient n_{H}=3, comparable to the reported literature values of 40-45 μM. The competitive inhibition of RecA ATPase activity was also investigated using this embodiment of the assay system, with ATPγS and ADP behaving essentially as expected and previously observed.

### Example 4

### Selection of RecA Activity with mRNA Display

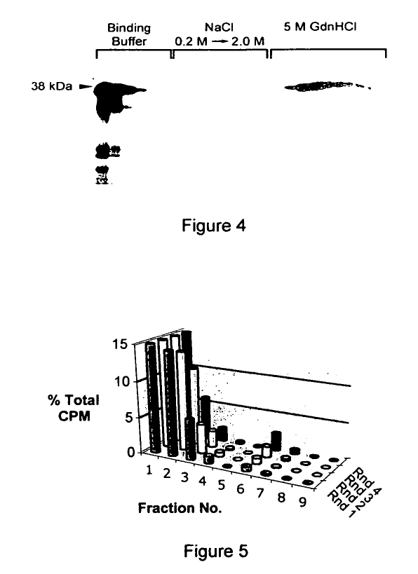

Methods for mRNA display in vitro protein selection have been developed by Liu and coworkers (Cho et al., 2000; Liu et al., 2000; and Baggio et al., 2002). In an initial test of the concept with regard to selecting proteins for interfering with RecA activity, purified RecA protein was biotinylated using the NHS ester of a commercial water-soluble biotin analog with a 32-Å tetraethylene oxide spacer arm (EZ-LINK™ NHS-PEO₄-Biotin, Pierce). Triplicate experiments demonstrated that 2.1±0.1 lysine residues were biotinylated per RecA monomer and that the nascent biotinylated RecA protein (B RecA) was efficiently immobilized: 4.3±0.2 μg bound to 50 μg of streptavidin-paramagnetic particles (SA-PMP). Importantly, the immobilized molecules were not released from the column after long-time incubations under the conditions used for binding and washing. The ^{B}RecA·SA-PMP complex (50 μg) was then used to bind ³⁵S-labeled RecA protein synthesized using in vitro transcription-translation. In agreement with the previous report of covalent RecA-agarose affinity columns (Freitag & McEntee, 1988), WT RecA protein could be pulled out of solution in the presence of Mg²⁺using ^{B}RecA·SA-PMP and subsequently eluted under denaturing conditions (FIG. 4).

### Example 5

### Individual mRNA-Displayed Members of a Random 20-Amino Acid Peptide are Selectively Retained and Amplified Using ^{B}RecA·SA-PMP

An mRNA displayed random peptide library was generated wherein each peptide contained approximately 20 randomized positions. To select RecA-binding peptides from such a library, the ^{B}RecA·SA-PMP strategy described above was used by immobilizing the target RecA protein on a solid surface followed by incubating with the mRNA displayed library. One issue is how to reduce background resulting from nonspecific binders. The biotin-streptavidin interaction, one of the strongest noncovalent interactions, was employed to immobilize RecA so that it can resist even harsh washing conditions. Based on previous reports that covalent RecA-agarose affinity columns released a bound 90-residue RecA fragment in the presence of 5 M guanidinium·HCl but not 2 M NaCl, high salt washes were used in the attempt to reduce non-specific background binding.

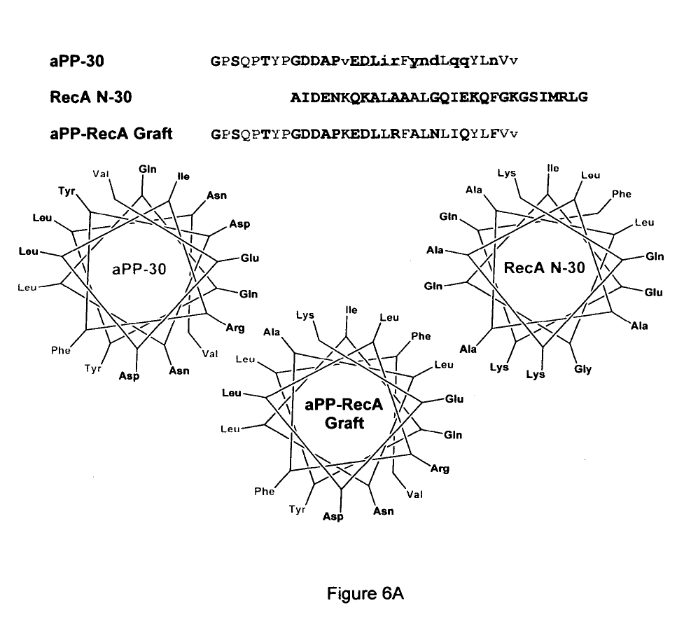

Four rounds of selection were performed using ^{B}RecA·SA-PMP (200 μg) with 1-mL in vitro transcription-translation reactions (1 μmol DNA), and the selection profiles are shown in FIG. 5. In the first two rounds of selection, the radioactive counts ([³⁵S]Met is included in the in vitro translation mix) from the elution flowthrough were not much higher than the background. The PCR amplified genetic material was used to re-generate a new mRNA displayed peptide library that was taken to the next round of selection. In round 3, however, approximately 1.5% of total immobilized radioactive counts were recovered in the eluant (FIG. 5, fraction 6). In round 4, an even higher fraction of the library was retained and selectively eluted. Two concerns were that either nonspecific binding or no binding would dominate the experiment. The first concern would have been manifest in a high fraction of retained peptides even in the earliest rounds, while the second concern would have appeared as no retained radioactivity. Since a low fraction of retained peptides that is slowly and monotonically increasing through round 4 was observed, it appears that the relatively rare peptides that bind RecA site-selectively were being amplified. 190 clones from the enriched fourth-round sequences were PCR amplified and cloned into TOPO vector for sequencing. The resulting peptide sequences were aligned and analyzed for identity and homology. Unfortunately, the occurrence of an overabundance of stretches of hydrophobic residues, particularly 3- and 4-residue runs of L and/or F, were observed that are indicative of nonspecific binding. While not providing the desired hit, these results do suggest that the mRNA peptide display technology platform can be used for the efficient selection and amplification of RecA-binding peptides from 10¹²-member random peptide libraries.

### Example 6

### RecA⁻ Bacteria Display a Significant Delay in the Development of Resistance to Antibiotics

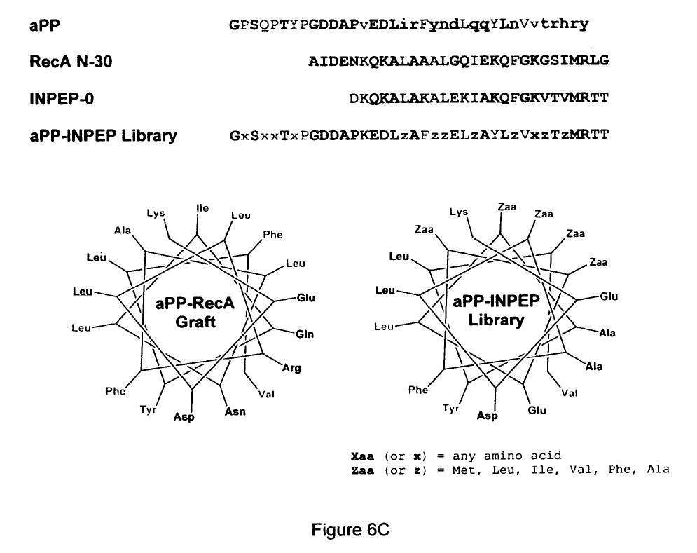

RecA controls the induction of enzymes responsible for mutational programs and recombinational events that allow horizontal gene transfer. Thus, its inhibition can potentiate the mutational response to antibiotics and reduce the rate of evolution of resistance. To confirm this, an experiment was performed wherein ca. 10,000 bacteria, either recA⁻ (ENZ280) or wild type (AB1157), were inoculated into media supplemented (or not) with chloramphenicol (FIG. 7a). By monitoring the OD₆₀₀ of the four cultures, the growth of the two different strains in the presence and absence of antibiotic was compared. Importantly, while the Rec⁺ strain was able to circumvent the antibiotic after several hours (antibiotic susceptibility tests confirmed that the culture was populated by carbenicillin-resistant clones), the recA⁻ was not able to overcome 5 μg/mL chloramphenicol, even after more than 50 h. Therefore, it can be concluded that RecA is required for efficient development of chloramphenicol resistance. Similar results were shown using carbenicillin, kanamycin, and ciprofloxacin.

Further, this study was extended with chloramphenicol to demonstrate a correlation between the speed at which chloramphenicol resistance develops and the level of SOS mutagenesis occurring (FIG. 7b). Not only is the mean latency time prior to resistance development dependent on RecA (in the absence of RecA, no resistance develops), but in the presence of RecA, antimutagens (spermidine) retards resistance development and mutagens (EMS) speed resistance development.

### Example 7

### N⁶-(1-Napthyl)-ADP Inhibits RecA-Dependent SOS Induction in Permeabilized E. Coli

As an initial test of the potential in vivo effects of ADP analogs 1-3, the use of bacterial cells rendered permeable to nucleotides was investigated. A number of techniques have been reported for bacterial permeabilization (see e.g., Scudamore et al., 1979; Bove et al., 1981; Vaara, 1992). A number of these techniques were utilized using propidium iodide (PI) in situ fluorescence as a metric of success (Niven & Mulholland, 1998). Bacterial cells are naturally impermeant to PI; however, appropriately permeabilized bacteria will take up PI allowing the formation of a highly fluorescent PI-DNA complex. Using this assay, it was found that treating cells with Epicentre Periplasting Kit buffer without lysozyme (200 mM Tris-HCl, pH 7.5,20% sucrose, 1 mM EDTA), followed by 0.1% Tween-80, provides a 100-fold increase in PI fluorescence. Moreover, bacterial cells treated in this way are viable and form colonies after treatment.

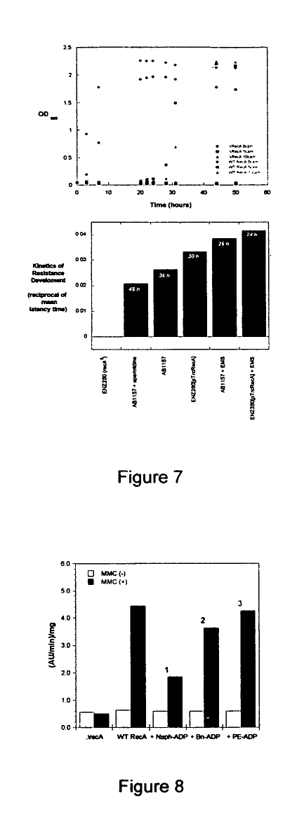

The influence of ADP analogs 1-3 on SOS induction by MMC in E. coli cells permeabilized as described above was tested. To evaluate the abilities of the nucleotide analogs to inhibit SOS induction, β-galactosidase activity was measured in GY7313 cells harboring a lacZ reporter gene fused to the sfi promoter, which is regulated by LexA protein. RecA-directed coproteolysis of LexA derepresses lacZ, and the β-galactosidase produced can be quantitatively measured by an established colorimetric assay (Berger et al., 2001). The extent of β-galactosidase induction is therefore an indirect measurement of the RecA protein's ability to form a nucleoprotein filament with coprotease activity. The results (FIG. 8) clearly demonstrate that 1 significantly inhibits SOS induction in permeabilized E. Coli cells.

### Example 8

### Bismuth-Dithiol Inhibition of RecA-Dependent SOS Induction and Synergism with Mitomycin C in E. Coli

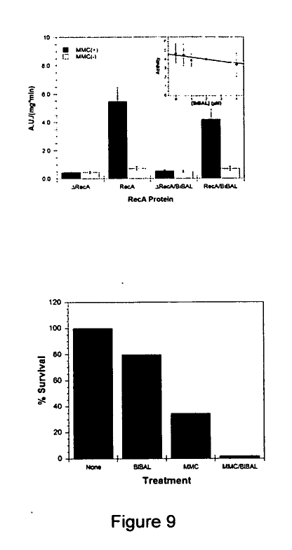

The effect of the BIBAL complex on RecA activity was tested using two different in vivo assays (FIG. 9). BiBAL appears to have a concentration-dependent effect on the level of SOS induction activity promoted by RecA in response to DNA damage (left graph and inset). Interestingly, BiBAL also appears to drastically sensitize recA⁺ cells to DNA damage (right graph). In the presence of MMC and BiBAL, cells only survive to <1% of the level of survival for untreated cells. As this effect is not as significant with the two agents acting alone, it appears that BiBAL and MMC act synergistically, suggesting that the DNA damage, which induces overexpression of RecA, is required for BiBAL to affect RecA. Similar results showing synergism between BiBAL and both Cipro and Norfloxacin were found.

### Example 9

### Construction of mRNA Displayed Random Peptide Library

An mRNA displayed peptide library is constructed according to the following procedures. First, a partially randomized DNA library is designed and synthesized containing 36 NNS codons. Second, this library is PCR amplified under normal conditions to introduce the left and right consensus sequence. The left consensus sequence contains a T7 promoter and a ΔTMV 5′ UTR to provide efficient in vitro transcription and translation. A FLAG tag is engineered at the very N terminus for purification or immunodetection of mRNA displayed peptides. The right arm contains a 6-His tag at the peptide's C terminus, followed by a short sequence for hybridizing/crosslinking with the puromycin-containing DNA linker. A sample of the PCR product is then cloned into a TOPO TA vector for sequencing. Third, the resulting dsDNA library is in vitro transcribed using T7 RNA polymerase. The mRNA templates with puromycin at 3′ ends are generated by psoralen-mediated crosslinking using an oligonucleotide containing a puromycin residue at its 3′ end. Translation is performed using rabbit reticulocyte lysate (Novagen, EMD Biosciences, Inc, an Affiliate of Merck, KG, Darmstadt, Germany.) and mRNA-peptide fusion formation is accomplished under optimized conditions (Liu et al., 2000). mRNA templates and mRNA-peptide fusions can then be isolated readily from the lysate using an oligo(dT) column, taking advantage of oligo(dA) residues at the puromycin-containing linker. To remove secondary RNA structures that might interfere with the functional selection step, the fusion molecules are converted to DNA/RNA hybrid by reverse transcription. These mRNA displayed proteins are then successively purified based on the FLAG affinity tag. The pre-selected library is characterized by sequencing 96 individually picked clones.

### Example 10

### Cellular Delivery of Nucleotide Analogs

Nucleotide analogs selected from high throughput screening (HTS) of chemical libraries likely need to be able to penetrate into the bacterial cytoplasm to be effective. While nucleosides are actively transported into the bacterium, anionic nucleotides tend to be impermeant. Two representative strategies that can be employed to address this situation are: (1) passive transport of phosphate ester “prodrug” of the desired nucleoside monophosphate; and (2) active transport of the desired nucleoside using bacteria heterologously expressing an indiscriminate nucleoside kinase. Both rely on the fact that nucleoside monophosphates can be further phosphorylated in situ by nonspecific nucleoside monophosphate kinases in E. coli (Blakley, 1983).

A nucleoside monophosphate is converted to a neutral “prodrug” by alkylating the phosphate moiety with a range of substituents (Krise & Stella, 1996). The neutral triesters passively permeate a bacterium where the alkyl groups are removed in situ by bacterial esterases or lipases. Corresponding phosphate triesters of candidate compounds are prepared according to reported methods (Krise & Stella, 1996; Kang et al., 1997; Kang & Cho, 1998; Kang et al., 1998).

As an alternative to the “prodrug” strategy, Herpes Simplex Virus thymidine kinase expressed as described by Loeb and coworkers (Kim & Loeb, 995) is employed. In this case, the unphosphorylated nucleoside analogs can be used directly.

In either case, the assay methods described elsewhere herein can demonstrate whether the analog is penetrating the cell. It should be noted that the use of nucleotide analogs can be accompanied by the use of its conjugate mutant RecA protein.

### Example 11

### Cellular Delivery of Peptides

Generally, natural peptides larger than four or five amino acids in size are not actively transported into bacteria by the oligopeptide transport machinery. Special sequences that confer bacterial cell permeability can, however, be directly incorporated into synthetic peptides. Several oligopeptide sequences have been reported that allow transport of peptides, proteins, and PNAs into bacterial cells (Good et al., 2001; Raiarao et al., 2002). For example, in some embodiments, the sequence CFFKDEL—recently show to deliver GFP in E. coli (Rajarao et al., 2002)—is appended to the N-terminus of a RecA-inhibiting peptide to direct the inhibitor peptide into the cell.

An alternative strategy is the direct expression of the desired peptide from a recombinant plasmid. Peptides corresponding to the N-terminal 77 (Horii et al., 1992) and 50 (Kiselev et al., 1988) residues of RecA have been shown to accumulate to functional levels in E. coli cells. The previously described culture conditions and specific genotypes employed (Horii et al., 1992; Kiselev et al., 1988) are thus employed to express RecA inhibitor peptides (e.g., RecA N-terminal mimetics). Alternatively, the peptide is expressed fused to a well-characterized soluble protein (e.g., maltose binding protein (Kapust & Waugh, 1999)) with an intervening self-cleaving intein (Singleton et al., 2002). The assay methods disclosed herein are utilized to demonstrate whether the peptide is accumulating within the bacteria.

### Example 12

### Determination of IRA Antibacterial Activity

An assay is utilized to determine whether an inhibitor of RecA activity (IRA) compound, whether nucleotide analog or peptide, is bacteriostatic or bactericidal utilizing, for example E. coli as a model test organism. A standard Kirby-Bauer disc method is used to determine the susceptibility of E. coli. Briefly, a homogeneous lawn of bacteria is produced within a thin layer of soft top agar across the surface of a culture plate by adding about 0.1 mL of a fresh overnight culture of E. coli (volume sufficient to reproduce the McFarland standard) to 2 mL of 45° C. melted top agar and pouring the suspension over the surface of a pre-warmed nutrient agar plate. After allowing the top agar to gel, a filter paper plug from a hole-punch is placed on the plate's surface and 5-10 μL of a solution comprising the putative antibacterial compound is pipetted onto the plug. The plates are incubated at 37° C. and any reduction in the turbidity of the lawn near the agent indicates inhibition of bacterial growth: the greater the antibacterial action, the wider the zone of inhibition (measured in mm). Thus, the antibacterial strength of the IRA can be judged by the width of the zone of inhibition around it.

As a quantitative measurement, the minimum inhibitory concentration (MIC) of synthetic compounds judged to have significant zones of inhibition is also determined. Fresh overnight cultures of E. coli are diluted to a turbidity of 0.02 OD₅₉₀ and added to the wells of a microtiter plate containing different antibiotic concentrations. The microplates are covered with gas-permeable sealing membrane and grown with shaking at 37° C. for 24 to 48 h. Turbidity is measured every few hours using a microplate reader.

### Example 13

### Determination of IRA Inhibition of SOS Induction

In order to determine if an IRA inhibits induction of the SOS response by RecA, a SOS chromotest is performed as previously described (Quillardet et al., 1982; Quillardet et al., 1993; Mersch-Sundermann et al., 1994; Vasilieva, 2002). This assay is performed using MMC in the absence and presence of a candidate compound. A negative chromotest in the presence of the candidate compound \ indicates inhibition of RecA activity.

### Example 14

### Characterization of IRA-Induced Phenotypes

In order to test directly whether bioactive IRAs inhibit RecA function, any one of several established in vivo assays can be employed that historically have been used in genetics experiments. For example, in addition to the UV survival and SOS induction assays described herein, assays are run to monitor the effects of the IRAs on high-frequency recombination (Konola et al., 1994) and Weigle reactivation of bacteriophage λ (Weigle, 1953; Radman, 1974) essentially as described. For each of the four phenotypic screens, assays performed in the presence and absence of small molecule are compared with one another as well as with control assays using recA⁻ bacteria.

### Example 15

### Monitoring Off-Target Effects

It can be desirable to characterize, at least qualitatively, the specificity of the compounds of the presently disclosed subject matter. In the case of the nucleotide analogs disclosed herein, the engineered bioorthogonality should minimize the problems associated with targeting at an ATP-binding site. In the case of the peptides disclosed herein, the sequence of the RecA N-terminal domain is not present elsewhere in the E. coli proteome and none of its interacting partners are reported to recognize the RecA N-terminal domain. Nevertheless, to be certain, the specificity of inhibitors of RecA's activities disclosed herein can be determined.

Comparison of the results of the in vivo assays described herein can provide certain information related to specificity. For example, if an IRA demonstrates antibacterial activity but no other activity clearly associated with RecA inhibition, it is reasonable to conclude that the effect is nonspecific. On the other hand, a non-bacteriotoxic candidate compound that gives phenotypic RecA screens results consistent with a RecA null phenotype, indicates that the compound's mode of action is specific to RecA.

In order to ascertain further whether a putative IRA has general toxicity problems, non-SOS stress response programs and general viability are monitored. The potential activation of stress response systems is tested using bacterial strains containing unique reporter gene fusions (e.g., GFP, DsRed, and luciferase) in each of three different stress response regulons (Gu & Choi, 2001; Gu & Gil, 2001; Kim & Gu, 2003; Kim et al., 2003; Lee & Gu, 2003). The effects of an IRA on general viability can be monitored using flow cytometry and confocal microscopy in conjunction with multiple fluorogenic metabolic labels (Banning et al., 2002; Herrera et al., 2002; Bartosch et al., 2003; Caruso et al., 2003; Creach et al., 2003; Gruden et al., 2003).

### Example 16

### Inhibition of Pseudomonas aeruginosa RecA

There are certain desirable qualities for particular organisms to have to be considered good targets for RecA inhibition. They must have a functional RecA homolog; that is, the organism must have a RecA-like protein that acts to repair damaged DNA and mediate SOS-like responses. In addition, in some embodiments, it can be advantageous for the heterologous organism's RecA-like protein to complement the activity of the native E. coli RecA protein by restoring the DNA repair functions and conferring viability in the presence of DNA damage to a ΔrecA E. coli strain. Matching this quality can speed up the process of screening heterologous RecA proteins with known assays in certain circumstances.

An organism possessing each of these characteristics is Pseudomonas aeruginosa, the bacterium responsible for chronic infection in the lungs in patients with cystic fibrosis. P. aeruginosa is an opportunistic bacterium, and in the lungs of a patient with cystic fibrosis, it embeds within mucosal secretions and proliferates, forming a biofilm that clogs the lungs and suffocates the patient. This pathogenicity of P. aeruginosa makes it an attractive target for an antibiotic regimen. In fact, several different antibiotic strategies are currently in use to combat Pseudomonas infection in cystic fibrosis patients, but unfortunately the bacteria thus far have continually evolving resistance to the treatments.

P. aeruginosa expresses a RecA protein that acts much like E. coli RecA. The P. aeruginosa RecA protein exhibits hyper-recombinogenic function in place of the E. coli RecA protein in recA⁻ E. coli cells and forms more stable complexes with DNA and ATP than the native E. coli protein (Namsaraev et al., 1998). In addition, chimeric proteins containing sequences from both E. coli and P. aeruginosa RecA proteins are recombination active, suggesting a complementation of the E. coli RecA function by the P. aeniginosa RecA (Bakhlanova et al., 2001). These results suggest that the P. aeruginosa RecA protein can be targeted for inhibition in much the same manner as the E. coli RecA protein has been targeted in some of the particular embodiments disclosed herein.

Given that the E. coli and P. aeruginosa RecA proteins cross-complement the other species, inhibition of P. aeruginosa can be approached in complementary ways. The inhibition of P. aeruginosa RecA heterologously expressed in E. coli is examined. Using techniques disclosed herein, inhibitors of P. aeruginosa RecA is identified.

### Example 17

### Inhibition of Neisseria gonorrhoeae RecA in Living Gonococci (GC)

A second organism matching the criteria described in Example 16 above is Neisseria gonorrhoeae (Stohl et al., 2002), a highly successful pathogen that colonizes the urogenital tract and is the sole etiological agent of the sexually transmitted disease (STD) gonorrhea. Members of the Neisseria genus have probably evolved with humans for thousands of years and have developed a system for varying surface molecules to evade the immune response. While the SOS response in Gc is clearly different from that of E. coli (Black et al., 1998) the Gc RecA plays crucial roles in in situ transformation (N. gonorrhoeae is naturally competent) and antigenic variation. The pathogenicity of Gc, coupled with the different relative importance of physiological roles of the Gc RecA, makes it an attractive target for an antibiotic strategy in accordance with the presently disclosed subject matter.

One experimental approach for inhibition of N. gonorrhoeae RecA tracks closely that described hereinabove for P. aeruginosa. An advantage of working with a Gc model is that there are trivial assays for RecA function in Gc that rely on the fact that its transformation is RecA-dependent. Thus, a reduction of the efficiency of transformation with a genetic element containing an antibiotic resistance marker reveals an inhibition of RecA function.

### Example 18

### Disovery of Peptides that Inhibit RecA Activities In Vitro by Selection from mRNA (Or Phase) Displayed Libraries

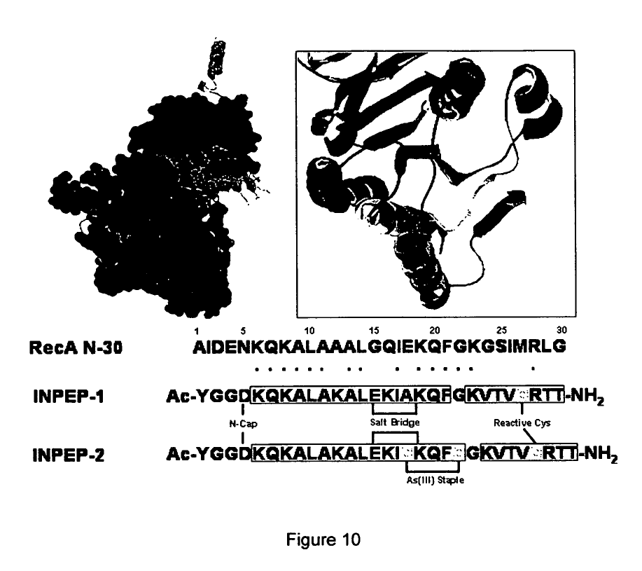

FIG. 10 shows the first and second inhibitory peptide (INPEP) designs. For INPEP-1, changes were made in the native peptide sequence (RecA N-30) that increased structural propensities of the helical and strand regions. The Tyr residue was added to the N-terminus with a Gly-Gly spacer to provide a simple spectral handle to aid quantitation. Asn was changed to an Asp to provide an N-cap and helix initiation locus. Ala12 and Gln16 were each changed to Lys to both increase solubility and positive character of the peptide. Gly15 was changed to Glu to propagate helicity and to provide an i to i+4 salt bridge with Lys19, which ultimately stabilizes the C-term of the helix. Glu18 was changed to Ala to decrease the unfavorable charge repulsion at the C-term of the helical dipole. Thr and Val residues have the highest propensity to be found in β-sheet structures, and therefore were incorporated into the sequence, patterned after the hydropathy of the native sequence (i.e. Ile→Val, Ser→Thr, etc.). Lastly, Met27 was changed to Cys, because in the crystal structure of the filament, this residue is directly across from Cys116 in the adjacent monomer. By making this change, a disulfide bond between the INPEP and the RecA monomer was created, thus irreversibly inhibiting the protein.

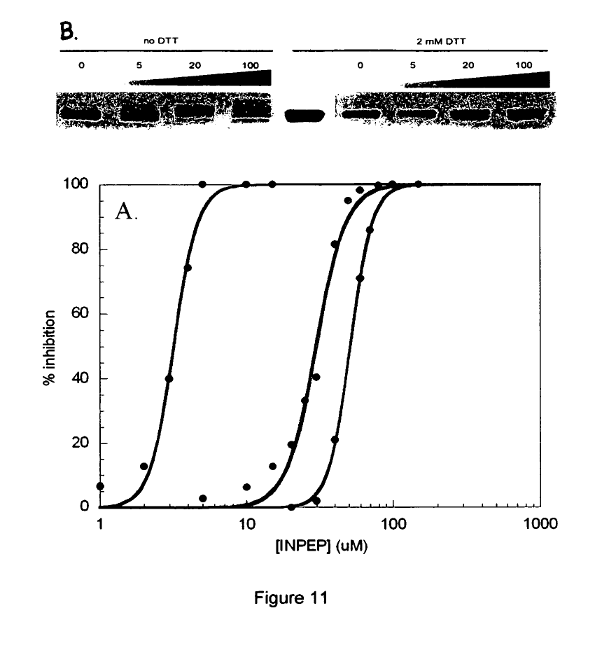

Because ATPase activity is associated with the active RecA-ATP-ssDNA complex, an in vitro assay for this activity was used to assess the effectiveness of the inhibitors, as disclosed herein. FIG. 11A shows the dose-dependent inhibition of RecA ATPase activity by reduced INPEP-1. The assay was run without reductant, dithiothreitol (DTT), present in the assay mixture to allow for the formation of disulfide bonds between peptide and protein. At the completion of the experiment, samples from the assay mixtures were separated by SDS-PAGE with and without prior DTT treatment in the loading buffer (FIG. 11B). A shift in the protein mobility is observed for each assay mixture inhibited with INPEP-1, which is reversed by addition of reductant prior to gel separation. A gel was run and used for in-gel chymotrypsin digestion and MALDI-MS identification of peptide fragments. The MS positively identified an INPEP-Cys116 fragment Derivatives of INPEP-1 were made to enhance disulfide reactivity or prevent it altogether. Iodoacetamide was reacted with the free thiol of INPEP-1 to make INPEP-1-SAc, which cannot form a disulfide with another free thiol. ATPase assays performed with and without DTT present yielded identical results and these were comparable to ATPase assays with rINPEP-1 with DTT present. The IC₅₀ for these inhibitory peptides were 40 μM and 33 μM for INPEP-1-SAc and rINPEP-1, respectively (FIG. 11A). The peptide was then “activated” by reaction under dilute conditions with an excess of the disulfide 2,2′-dithiodipyridine, which undergoes thiol-disulfide exchange with peptide thiols to form a highly oxidizing thiopyridyl disulfide. ATPase assays performed under non-reducing conditions yielded an IC₅₀ of this activated peptide, INPEP-1-TP, of only 5 μM (FIG. 1 1A). By activation of the reactive cysteine (see FIG. 10), the potency of INPEP-1 was increased by a factor of 10.

Having found two micromolar peptide inhibitors of RecA by rational design, the design can be further improved by randomizing the interfacial amino acids and selecting for the tightest binders. This can be accomplished using mRNA display. A library of 10¹² peptides that mimic the structure of the RecA N-terminal domain can be generated and screened for interaction with the core domain of the RecA protein. From there, the mRNA can be recovered that encodes the peptide and its specific sequence determined. Synthesis and purification of the peptide can allow the interactions between RecA and the peptide to be observed and characterized in vitro.

The central feature of this method is that the nascent peptide is covalently displayed on the 3′ end of its own mRNA. Since the genotype (coding sequence) and the phenotype (polypeptide sequence) are united in a single molecule, mRNA display provides a powerful means for rapidly reading and amplifying a protein sequence after it has been selected from a library based on its function. Multiple rounds of selection and amplification are carried out, enabling the enrichment and isolation of very rare molecules. Compared to other protein selection strategies, mRNA display provides a number of significant advantages that can make it well suited to the identification of site-specific RecA-binding peptides.

To apply mRNA display in addressing biochemical problems of site-specific RecA binding with high specificity, a technology platform can be developed that allows immobilization of RecA protein on a solid surface (SA-PMP) via biotin residues conjugated to each monomer. Upon incubation with the mRNA displayed peptide library of interest, peptide sequences that are specifically bound can be released and enriched, with the intact mRNA still covalently attached to the C-terminus of each peptide. The selected sequences can be amplified and used as templates to re-generate the mRNA displayed peptide library for iterative rounds of selection. If the need arises to evolve the peptide of interest with desired properties, the library can be generated based on the sequence of the best peptides from prior rounds using error prone PCR, DNA shuffling, or other mutagenesis methods.

Construction of mRNA displayed partially randomized peptide library The mRNA displayed peptide library can be constructed according to the following procedures. First, DNA cassettes can be synthesized with degenerate codons encoding five hydrophobic amino acids in the seven interfacial positions. The degenerate codon NTN, where N is any DNA nucleotide, can encode for amino acids Met, Leu, Ile, Val, and Phe. The remaining static positions can be the residues of INPEP-1, and can be encoded by the MAX codons for optimal expression in E. coli. Second, this library can be PCR amplified under normal conditions to introduce the left and right consensus sequence. The left consensus sequence contains a T7 promoter and a TMV 5′ UTR to provide efficient in vitro transcription and translation. A FLAG tag can be engineered at the very N terminus for purification or immunodetection of mRNA displayed peptides. The right arm can contain a 6-His tag at the peptide's C terminus, followed by a short sequence for hybridizing/crosslinking with the puromycin-containing DNA linker. A sample of the PCR product can then be cloned into a TOPO TA vector for sequencing. Third, the resulting dsDNA library can be in vitro transcribed using T7 RNA polymerase. The mRNA templates with puromycin at 3′ ends can be generated by psoralen-mediated crosslinking using an oligonucleotide containing a puromycin residue at its 3′ end. Translation is performed using rabbit reticulocyte lysate and mRNA-peptide fusion formation is accomplished under optimized conditions. mRNA templates and mRNA-peptide fusions are then isolated readily from the lysate using an oligo(dT) column, taking advantage of oligo(dA) residues at the puromycin-containing linker. To remove secondary RNA structures that might interfere with the functional selection step, the fusion molecules can be converted to DNA/RNA hybrid by reverse transcription. These mRNA displayed proteins can then be purified based on the FLAG affinity tag. The pre-selected library can be characterized by sequencing 96 individually picked clones.

Sequence analysis of enriched pools of displayed peptides Once the threshold fraction of total radioactivity is present in the eluted fractions, the sequences of these peptides can be determined and analyzed.

Typically, two microplates (190 individual clones) can be used for sequencing and analysis.

In vitro analysis of RecA binding by isolated peptides As patterns in the sequence data begin to emerge, interesting individual clones can be selected for further analysis. Individual purified plasmids can be used as the template for isolated in vitro transcription-translation (TNT™) reactions, the peptide products can be purified using the FLAG tag, and then titrated into wells containing ^{B}RecA-SA-PMP. Monitoring ³⁵S retained as a function of the titer will provide rough estimates of relative dissociation constants. These values can be used to identify the peptide sequences most likely to be avid inhibitors of RecA in solution.

Characterization of inhibition of RecA activities by purified peptides Once the sequence analysis, verified by in vitro binding experiments, has provided a picture of key elements for site-specific RecA binding, individual peptides can be chemically synthesized and purified. The purified peptides can be used as potential inhibitors in the assays described herein.

In Vitro Characterization of Purified Peptides and Their Interactions with RecA

A peptide (or peptides) that is a reasonably tight-binding inhibitor of RecA activities can be characterized to ascertain its structure and whether it binds to the RecA core domain's “acceptor” region as designed. First, peptide secondary and tertiary structure can be determined using CD spectroscopy and NMR spectrometry, respectively. In order to determine whether the peptide binds and inhibits as designed, chemical shift perturbation and transfer NOE experiments using isotopically labeled ΔN33 RecA can be performed.

Design Via Arsenic-Binding Staple

As disclosed herein, the first design of inhibitory peptide-1 (INPEP-1) resulted in an IC₅₀ of 33 μM. Briefly, the design of INPEP-1 was based on residues of the N-terminal helix-loop-sheet domain of the RecA involved in intermonomer contact in the inactive filament crystal structure. Residues not involved in monomer-monomer contact were rationally redesigned to residues with improved structural propensities. The design also involved a Met→Cys mutation at position 27 to provide a reactive group to covalently lock the inhibitor to the target protein via disulfide bond formation, a strategy which provided a ten-fold improvement on efficacy.

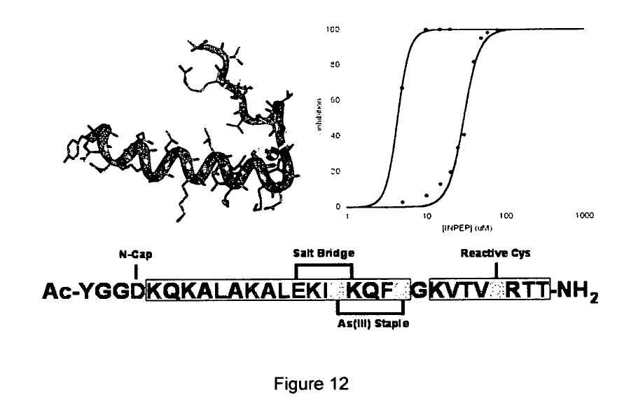

As an alternative to peptide selection from in vitro libraries, a second sequence has been designed (INPEP-2), which has an additional structural element to improve helicity. FIG. 12 shows the arsenic-binding “staple” at the C-term of the α-helix. Previous work has shown that dicysteines in i & i+4 positions at the C-terminal pole of an α-helix can increase the helical character by roughly 14% (ΔΔG≈−1 kcal/mol) with monomethyl arsonous acid bound.

The incorporation of two additional cysteines into the sequence requires special consideration in synthetic scheme of this peptide. For the chelating cysteines, acid-labile trityl protecting groups can be used, and for the protein-reactive cysteine, the orthogonal t-butylthiol disulfide protecting group can be used. The peptide can be cleaved from the solid support and concomitantly side-chain deprotected with trifluoroacetic acid and non-thiol scavengers anisole, water, and triisopropylsilane. A slight excess of monomethyl arsonous acid can be added to the cleaved peptide. The arsenic-stabilized peptide can then be purified by RP-HPLC, concentrated by lyophilization, redissolved in 0.1 M ammonium acetate (pH 8.0) with a 10-fold excess of the disulfide reductant, tris-(2-carboxyethyl) phosphine (TCEP). After complete reduction of the reactive cysteine's t-butylthiol disulfide, the peptide can again be purified by RP-HPLC, lyophilized, and stored under argon to prevent intermolecular disulfide formation and arsenic shuffling.

A batch of this peptide design has been made and tested in the ATPase inhibition assay in the absence of DTT. The initial results were promising, with an IC₅₀ of about 4 μM, a 10-fold increase in potency over the comparable INPEP-1 design (see FIG. 12). The thiopyridine-activated INPEP-2 can be synthesized and tested for its in vitro RecA ATPase activity inhibition, which is expected to be greater than the reduced parent peptide. Since INPEP-2 is designed to have greater helical content than INPEP-1, the increase in helicity due to the arsenic staple can be quantitated, and the increased structural stability related to the increased ability to inhibit RecA activity.

### REFERENCES

The references listed below as well as all references cited in the specification are incorporated herein by reference to the extent that they supplement, explain, provide a background for or teach methodology, techniques and/or compositions employed herein.
Adams, S. R., Campbell, R. E., Gross, L. A., Martin, B. R., Walkup, G. K., Yao, Y., Llopis, J. & Tsien, R. Y. (2002). New biarsenical ligands and tetracysteine motifs for protein labeling in vitro and in vivo: synthesis and biological applications. J Am Chem Soc 124, 6063-76.
Aronov, A. M., Suresh, S., Buckner, F. S., Van Voorhis, W. C., Verlinde, C. L., Opperdoes, F. R., Hol, W. G. & Gelb, M. H. (1999). Structure-based design of submicromolar, biologically active inhibitors of trypanosomatid glyceraldehyde-3-phosphate dehydrogenase. Proc Natl Acad Sci U S A 96, 4273-8.
Baggio, R., Burgstaller, P., Hale, S. P., Putney, A. R., Lane, M., Lipovsek, D., Wright, M. C., Roberts, R. W., Liu, R., Szostak, J. W. & Wagner, R. W. (2002). Identification of epitope-like consensus motifs using mRNA display. J Mol Recognit 15, 126-34.
Baker, W. R., Cai, S., Dimitroff, M., Fang, L., Huh, K. K., Ryckman, D. R., Shang, X., Shawar, R. M. & Therrien, J. H. (2004). A prodrug approach toward the development of water soluble fluoroquinolones and structure—activity relationships of quinoline-3-carboxylic acids. J Med Chem 47, 4693-709.
Bakhlanova, I. V., Ogawa, T. & Lanzov, V. A. (2001). Recombinogenic activity of chimeric recA genes (Pseudomonas aeruginosa/Escherichia coli): a search for RecA protein regions responsible for this activity. Genetics 159, 7-15.
Banning, N., Toze, S. & Mee, B. J. (2002). Escherichia coli survival in groundwater and effluent measured using a combination of propidium iodide and the green fluorescent protein. J Appl Microbiol 93, 69-76.
Bartosch, S., Mansch, R., Knotzsch, K. & Bock, E. (2003). CTC staining and counting of actively respiring bacteria in natural stone using confocal laser scanning microscopy. J Microbiol Methods 52, 75-84.
Berger, M. D., Lee, A. M., Simonette, R. A., Jackson, B. E., Roca, A. I. & Singleton, S. F. (2001). Design and evaluation of a tryptophanless RecA protein with wild type activity. Biochem Biophys Res Commun 286, 1195-203.
Bisognano, C., Kelley, W. L., Estoppey, T., Francois, P., Schrenzel, J., Li, D., Lew, D. P., Hooper, D. C., Cheung, A. L. & Vaudaux, P. (2004). A recA-LexA-dependent pathway mediates ciprofloxacin-induced fibronectin binding in Staphylococcus aureus. J Biol Chem 279, 9064-71.
Blakley, R. L. (1983). Nucleotide metabolism. In Biochemsitry, 1st ed. (Zubay, G., ed.), pp. 713-714. Addison-Wesley Publishing Company, Inc., London.

Boye, E., Alver, S. & Skarstad, K. (1981). Deoxyribonucleic acid replication in permeable and fully viable Escherichia coli cells. J Bacteriol 145, 1413-6.
Brenner, S. L., Mitchell, R. S., Morrical., S. W., Neuendorf, S. K., Schutte, B. C. & Cox, M. M. (1987). recA protein-promoted ATP hydrolysis occurs throughout recA nucleoprotein filaments. J Biol Chem 262, 4011-6.
Bressi, J. C., Choe, J., Hough, M. T., Buckner, F. S., Van Voorhis, W. C., Verlinde, C. L., Hol, W. G. & Gelb, M. H. (2000). Adenosine analogues as inhibitors of Trypanosoma brucei phosphoglycerate kinase: elucidation of a novel binding mode for a 2-amino-N(6)-substituted adenosine. J Med Chem 43, 4135-50.
Bressi, J. C., Verlinde, C. L., Aronov, A. M., Shaw, M. L., Shin, S. S., Nguyen, L. N., Suresh, S., Buckner, F. S., Van Voorhis, W. C., Kuntz, I. D., Hol, W. G. & Gelb, M. H. (2001). Adenosine analogues as selective inhibitors of glyceraldehyde-3-phosphate dehydrogenase of Trypanosomatidae via structure-based drug design. J Med Chem 44, 2080-93.
Briand, G. G. & Burford, N. (1999). Bismuth compounds and preparations with biological or medicinal relevance. Chem Rev 99, 2601-58.
Cairns, J., Overbaugh, J. & Miller, S. (1988). The origin of mutants. Nature 335, 142-5.

Capaldo, F. N. & Barbour, S. D. (1975). The role of the rec genes in the viability of Escherichia coli K12. Basic Life Sci 5A, 405-18.
Caruso, G., Mancuso, M. & Crisafi, E. (2003). Combined fluorescent antibody assay and viability staining for the assessment of the physiological states of Escherichia coli in seawaters. J Appl Microbiol 95, 225-33.
Chene, P. (2002). ATPases as drug targets: learning from their structure. Nat Rev Drug Discov 1, 665-73.
Chervyakova, D., Kagansky, A., Petukhov, M. & Lanzov, V. (2001). [L29M] substitution in the interface of subunit-subunit interactions enhances Escherichia coli RecA protein properties important for its recombinogenic activity. J Mol Biol 314, 923-35.

Cho, G., Keefe, A. D., Liu, R., Wilson, D. S. & Szostak, J. W. (2000). Constructing high complexity synthetic libraries of long ORFs using in vitro selection. J Mol Biol 297, 309-19.
Clark, A. J. & Margulies, A. D. (1965). Isolation and Characterization of Recombination-Deficient Mutants of Escherichia Coli K22. Proc Natl Acad Sci U S A 53, 451-9.
Courcelle, J., Khodursky, A., Peter, B., Brown, P. O. & Hanawalt, P. C. (2001). Comparative gene expression profiles following UV exposure in wild-type and SOS-deficient Escherichia coli. Genetics 158, 41-64.
Cox, M. M., Goodman, M. F., Kreuzer, K. N., Sherratt, D. J., Sandler, S. J. & Marians, K. J. (2000). The importance of repairing stalled replication forks. Nature 404, 37-41.
Creach, V., Baudoux, A. C., Bertru, G. & Rouzic, B. L. (2003). Direct estimate of active bacteria: CTC use and limitations. J. Microbiol Methods 52, 19-28.
DasGupta, C., Shibata, T., Cunningham, R. P. & Radding, C. M. (1980). The topology of homologous pairing promoted by RecA protein. Cell 22, 437-46.
Davisson, V. J., Davis, D. R., Dixit, V. M. & Poulter, C. D. (1987). Synthesis of nucleotide 5′-diphosphates from 5′-O-tosyl nucleosides. Journal of Organic Chemistry 52, 1794-801.
Désaubry, L., Shoshani, I., & Johnson, R. A. (1996). Inhibition of adenyl cyclase by a family of newly synthesized adenine nucleoside 3′-polyphosphates. J. Biol. Chem. 271, 14028-14034.

Dimpfl, J. & Echols, H. (1989). Duplication mutation as an SOS response in Escherichia coli: enhanced duplication formation by a constitutively activated RecA. Genetics 123, 255-60.

Domenico, P., Baldassarri, L., Schoch, P. E., Kaehier, K., Sasatsu, M. & Cunha, B. A. (2001). Activities of bismuth thiols against staphylococci and staphylococcal biofilms. Antimicrob Agents Chemother 45, 1417-21.
Domenico, P., Salo, R. J., Novick, S. G., Schoch, P. E., Van Horn, K. & Cunha, B. A. (1997). Enhancement of bismuth antibacterial activity with lipophilic thiol chelators. Antimicrob Agents Chemother 41, 1697-703.
Domenico, P., Tomas, J. M., Merino, S., Rubires, X. & Cunha, B. A. (1999). Surface antigen exposure by bismuth dimercaprol suppression of Klebsiella pneumoniae capsular polysaccharide. Infect Immun 67, 664-9.
Epple, R., Kudirka, R. & Greenberg, W. A. (2003). Solid-phase synthesis of nucleoside analogues. J Comb Chem 5, 292-310.
Freitag, N. & McEntee, K. (1988). Affinity chromatography of RecA protein and RecA nucleoprotein complexes on RecA protein-agarose columns. J Biol Chem 263, 19525-34.
Gillespie, P. G., Gillespie, S. K., Mercer, J. A., Shah, K. & Shokat, K. M. (1999). Engineering of the myosin-ibeta nucleotide-binding pocket to create selective sensitivity to N(6)-modified ADP analogs. J Biol Chem 274, 31373-81.
Good, L., Awasthi, S. K., Dryselius, R., Larsson, O. & Nielsen, P. E. (2001). Bactericidal antisense effects of peptide-PNA conjugates. Nat Biotechnol 19, 360-4.
Good, L., Awasthi, S. K., Dryselius, R., Larsson, O. & Nielsen, P. E. (2001). Bactericidal antisense effects of peptide-PNA conjugates. Nat Biotechnol 19, 360-4.
Goodman, M. F. (2000). Coping with replication ‘train wrecks’ in Escherichia coli using Pol V, Pol II and RecA proteins. Trends Biochem Sci 25, 189-95.; Hastings, P. J. & Rosenberg, S. M. (2002). In pursuit of a molecular mechanism for adaptive gene amplification. DNA Repair (Amst) 1, 111-23.
Gruden, C. L., Khijniak, A. & Adriaens, P. (2003). Activity assessment of microorganisms eluted from sediments using 5-cyano-2,3-ditolyl tetrazolium chloride: a quantitative comparison of flow cytometry to epifluorescent microscopy. J Microbiol Methods 55, 865-74.
Gu, M. B. & Choi, S. H. (2001). Monitoring and classification of toxicity using recombinant bioluminescent bacteria. Water Sci Technol 43, 147-54.

Gu, M. B. & Gil, G. C. (2001). A multi-channel continuous toxicity monitoring system using recombinant bioluminescent bacteria for classification of toxicity. Biosens Bioelectron 16, 661-6.

Hanessian, S. & Huynh, H. K. (1999). Formation of 4-alkoxybenzylidene acetals on solid support and generation of functional diversity with carbohydrate scaffolds. Synlett, 102-104.
Harris, D. A. (1987). In Spectrophotometry and Spectrofluorometry (Bashford, C. L. a. H., D. A., ed.), pp. 49-90. IRL Press, Washington, D.C.
He, J. & Seela, F. (2002). Propynyl groups in duplex DNA: stability of base pairs incorporating 7-substituted 8-aza-7-deazapurines or 5-substituted pyrimidines. Nucleic Acids Res 30, 5485-96.
Herrera, G., Martinez, A., Blanco, M. & O'Connor, J. E. (2002). Assessment of Escherichia coli B with enhanced permeability to fluorochromes for flow cytometric assays of bacterial cell function. Cytometry 49, 62-9.
Hiom, K. J. & Sedgwick, S. G. (1992). Alleviation of EcoK DNA restriction in Escherichia coli and involvement of umuDC activity. Mol Gen Genet 231, 265-75.
Horii, T., Ozawa, N., Ogawa, T. & Ogawa, H. (1992). Inhibitory effects of N- and C-terminal truncated Escherichia coli recA gene products on functions of the wild-type recA gene. J Mol Biol 223, 105-14.
Horii, T., Ozawa, N., Ogawa, T. & Ogawa, H. (1992). Inhibitory effects of N- and C-terminal truncated Escherichia coli recA gene products on functions of the wild-type recA gene. J Mol Biol 223, 105-14.
Horwitz, J. P., Urbanski, J. A. & Chura, J. (1962). Nucleosides. II. 5′-O-Mesylthymidine and 3′-O-mesylthymidine. Journal of Organic Chemistry 27, 3300-2.
Huang, C. T. & Stewart, P. S. (1999). Reduction of polysaccharide production in Pseudomonas aeruginosa biofilms by bismuth dimercaprol (BisBAL) treatment. J Antimicrob Chemother 44, 601-5.
Janion, C. (2001). Some aspects of the SOS response system—a critical survey. Acta Biochim Pol 48, 599-610.

Kang, S. H. & Cho, M. J. (1998). Biological activity and phosphorylation of 2′-azido-2′-deoxyuridine and 2′-azido-2′-deoxycytidine. Nucleosides Nucleotides 17, 1077-88.
Kang, S. H., Sinhababu, A. K. & Cho, M. J. (1998). Synthesis and biological activity of bis(pivaloyloxymethyl) ester of 2′-azido-2′-deoxyuridine 5′-monophosphate. Nucleosides Nucleotides 17, 1089-98.
Kang, S. H., Sinhababu, A. K., Cory, J. G., Mitchell, B. S., Thakker, D. R. & Cho, M. J. (1997). Cellular delivery of nucleoside diphosphates: a prodrug approach. Pharm Res 14, 706-12.
Kapoor, T. M. & Mitchison, T. J. (1999). Allele-specific activators and inhibitors for kinesin. Proc Natl Acad Sci U S A 96, 9106-11.
Kapust, R. B. & Waugh, D. S. (1999). Escherichia coli maltose-binding protein is uncommonly effective at promoting the solubility of polypeptides to which it is fused. Protein Sci 8, 1668-74.
Kapust, R. B. & Waugh, D. S. (1999). Escherichia coli maltose-binding protein is uncommonly effective at promoting the solubility of polypeptides to which it is fused. Protein Sci 8,1668-74.
Kaye, K. S., Fraimow, H. S. & Abrutyn, E. (2000). Pathogens resistant to antimicrobial agents. Epidemiology, molecular mechanisms, and clinical management. Infect Dis Clin North Am 14, 293-319.
Khil, P. P. & Camerini-Otero, R. D. (2002). Over 1000 genes are involved in the DNA damage response of Escherichia coli. Mol Microbiol 44, 89-105.
Kiessling, L. L., Griffin, L. C. & Dervan, P. B. (1992). Flanking sequence effects within the pyrimidine triple-helix motif characterized by affinity cleaving. Biochemistry 31, 2829-34.
Kikugawa, K., lizuka, K., & Ichino, M. (1973). Platelet aggregation inhibitors. 4. N 6-substituted adenosines. J. Med Chem. 16, 358-364.
Kim, B. & Loeb, L. A. (1995). A screen in Escherichia coli for nucleoside analogs that target human immunodeficiency virus (HIV) reverse transcriptase: coexpression of HIV reverse transcriptase and herpes simplex virus thymidine kinase. J Virol 69, 6563-6.
Kim, B. C. & Gu, M. B. (2003). A bioluminescent sensor for high throughput toxicity classification. Biosens Bioelectron 18, 1015-21.
Kim, B. C., Park, K. S., Kim, S. D. & Gu, M. B. (2003). Evaluation of a high throughput toxicity biosensor and comparison with.a Daphnia magna bioassay. Biosens Bioelectron 18, 821-6.
Kiselev, V. I., Glukhov, A. I., Tarasova, I. M. & Shchepetov, M. E. (1988). Accumulation of N-terminal fragment of recA protein in the htpR-mutant impairs the SOS-function of Escherichia coli cells. Mol Biol (Mosk) 22, 1198-203.
Konola, J. T., Logan, K. M. & Knight, K. L. (1994). Functional characterization of residues in the P-loop motif of the RecA protein ATP binding site. J. Mol. Biol. 237, 20-34.
Krise, J. P. & Stella, V. J. (1996). Prodrugs of phosphates, phosphonates, and phosphinates. Advanced Drug Delivery Reviews 19, 287-310.
Kuan, C. T., Liu, S. K. & Tessman, I. (1991). Excision and transposition of Tn5 as an SOS activity in Escherichia coli. Genetics 128, 45-57.
Larminat, F. & Defais, M. (1989). Modulation of the SOS response by truncated RecA proteins. Mol. Gen. Genet. 216, 106-112.
Lee, A. M. & Singleton, S. F. (2004). Inhibition of the Escherichia coli RecA protein: zinc(II), copper(II) and mercury(II) trap RecA as inactive aggregates. J Inorg Biochem 98, 1981-6.
Lee, H. J. & Gu, M. B. (2003). Construction of a sodA::IuxCDABE fusion Escherichia coli: comparison with a katG fusion strain through their responses to oxidative stresses. Appl Microbiol Biotechnol 60, 577-80.
Lee, S. H. & Kohn, H. (2004). Cyclic disulfide C8 iminoporfiromycin: nucleophilic activation of a porfiromycin. J Am Chem Soc 126, 4281-92.
Lee, S. H. & Kohn, H. (2005). 7-N,7[prime or minute]-N[prime or minute]-(1[double prime],2[double prime]-Dithianyl-3[double prime],6[double prime]-dimethylenyl)bismitomycin C: synthesis and nucleophilic activation of a dimeric mitomycin. Org Biomol Chem 3, 471-82.
Levy, M. S., Balbinder, E. & Nagel, R. (1993). Effect of mutations in SOS genes on UV-induced precise excision of Tn10 in Escherichia coli. Mutat Res 293, 241-7.
Liu, R., Barrick, J. E., Szostak, J. W. & Roberts, R. W. (2000). Optimized synthesis of RNA-protein fusions for in vitro protein selection. Methods Enzymol 318, 268-93.
Lusetti, S. L., Shaw, J. J. & Cox, M. M. (2003). Magnesium ion-dependent activation of the RecA protein involves the C terminus. J Biol Chem 278, 16381-8.
Mahony, D. E., Lim-Morrison, S., Bryden, L., Faulkner, G., Hoffman, P. S., Agocs, L., Briand, G. G., Burford, N. & Maguire, H. (1999). Antimicrobial activities of synthetic bismuth compounds against Clostridium difficile. Antimicrob Agents Chemother 43, 582-8.
Mao, L., Wang, Y., Liu, Y. & Hu, X. (2004). Molecular determinants for ATP-binding in proteins: a data mining and quantum chemical analysis. J Mol Biol 336, 787-807.
Matic, I., Rayssiguier, C. & Radman, M. (1995). Interspecies gene exchange in bacteria: the role of SOS and mismatch repair systems in evolution of species. Cell 80, 507-15.
Menge, K. L. & Bryant, F. R. (1988). ATP-stimulated hydrolysis of GTP by RecA protein: kinetic consequences of cooperative RecA protein-ATP interactions. Biochemistry 27, 2635-2640.
Mersch-Sundermann, V., Schneider, U., Klopman, G. & Rosenkranz, H. S. (1994). SOS induction in Escherichia coli and Salmonella mutagenicity: a comparison using 330 compounds. Mutagenesis 9, 205-24.
Metzker, M. L., Raghavachari, R., Richards, S., Jacutin, S. E., Civitello, A., Burgess, K. & Gibbs, R. A. (1994). Termination of DNA synthesis by novel 3′-modified-deoxyribonucleoside 5′-triphosphates. Nucleic Acids Res 22, 4259-67.
Mikawa, T., Masui, R., Ogawa, T., Ogawa, H. & Kuramitsu, S. (1995). N-terminal 33 amino acid residues of Escherichia coli RecA protein contribute to its self-assembly. J Mol Biol 250, 471-83.
Morrical., S. W., Lee, J. & Cox, M. M. (1986). Continuous association of Escherichia coli single-stranded DNA binding protein with stable complexes of recA protein and single-stranded DNA. Biochemistry 25, 1482-94.
Mushegian, A. R. & Koonin, E. V. (1996). A minimal gene set for cellular life derived by comparison of complete bacterial genomes. Proc Natl Acad Sci U S A 93, 10268-73.
Na, Y., Wang, S. & Kohn, H. (2002). 7-N-(mercaptoalkyl)mitomycins: implications of cyclization for drug function. J Am Chem Soc 124, 4666-77.
Namsaraev, E. A., Baitin, D., Bakhlanova, I. V., Alexseyev, A. A., Ogawa, H. & Lanzov, V. A. (1998). Biochemical basis of hyper-recombinogenic activity of Pseudomonas aeruginosa RecA protein in Escherichia coli cells. Mol Microbiol 27, 727-38.
Niven, G. W. & Mulholland, F. (1998). Cell membrane integrity and lysis in Lactococcus lactis: the detection of a population of permeable cells in post-logarithmic phase cultures. J Appl Microbiol 84, 90-6.
Oikawa, Y., Yoshioka, T. & Yonemitsu, O. (1982). Protection of hydroxy groups by intramolecular oxidative formation of methoxybenzylidene acetals with DDQ. Tetrahedron Letters 23, 889-92.
Pillai, S. P., Pillai, C. A., Shankel, D. M. & Mitscher, L. A. (2001). The ability of certain antimutagenic agents to prevent development of antibiotic resistance. Mutat Res 496, 61-73.
Quillardet, P. & Hofnung, M. (1993). The SOS chromotest: a review. Mutat Res 297, 235-79.
Quillardet, P., Huisman, O., D'Ari, R. & Hofnung, M. (1982). SOS chromotest, a direct assay of induction of an SOS function in Escherichia coli K-12 to measure genotoxicity. Proc Natl Acad Sci U S A 79, 5971-5.
Radman, M. (1974). Phenomenology of an inducible mutagenic DNA repair pathway in Escherichia coli: SOS repair response hypothesis. In Molecular and Environmental Aspects of Mutagenesis (Sherman, S., Miller, M., Lawrence, C. & Tabor, W. H., eds.). Charles C. Thomas Publisher, Springfield.
Radman, M., Taddei, F. & Matic, I. (2000). DNA Repair Systems and Bacterial Evolution. Cold Spring Harb Symp Quant Biol 65, 11-19.
Rajarao, G. K., Nekhotiaeva, N. & Good, L. (2002). Peptide-mediated delivery of green fluorescent protein into yeasts and bacteria. FEMS Microbiol Lett 215, 267-72.
Rajarao, G. K., Nekhotiaeva, N. & Good, L. (2002). Peptide-mediated delivery of green fluorescent protein into yeasts and bacteria. FEMS Microbiol Lett 215, 267-72.
Rinken, R. & Wackernagel, W. (1992). Inhibition of the recBCD-dependent activation of Chi recombinational hot spots in SOS-induced cells of Escherichia coli. J Bacteriol 174, 1172-8.
Robey, M., Benito, A., Hutson, R. H., Pascual., C., Park, S. F. & Mackey, B. M. (2001). Variation in resistance to high hydrostatic pressure and rpoS heterogeneity in natural isolates of Escherichia coli O157:H7. Appl Environ Microbiol 67, 4901-7.
Roca, A. I. & Cox, M. M. (1997). RecA protein: structure, function, and role in recombinational DNA repair. Prog Nucleic Acid Res Mol Biol 56, 129-223.
Roca, A. I. & Singleton, S. F. (2003). Direct evaluation of a mechanism for activation of the RecA nucleoprotein filament. J Am Chem Soc 125, 15366-75.
Rodenko, B., Wanner, M. J. & Koomen, G.-J. (2002). Solid phase synthesis of C2,N6-disubstituted adenosine analogues. Journal of the Chemical Society, Perkin Transactions 1, 1247-1252.
Sassanfar, M. & Roberts, J. W. (1990). Nature of the SOS-inducing signal in Escherichia coli. The involvement of DNA replication. J Mol Biol 212, 79-96.
Sassanfar, M. & Roberts, J. W. (1990). Nature of the SOS-inducing signal in Escherichia coli. The involvement of DNA replication. J Mol Biol 212, 79-96.
Scudamore, R. A., Beveridge, T. J. & Goldner, M. (1979). Outer-membrane penetration barriers as components of intrinsic resistance to beta-lactam and other antibiotics in Escherichia coli K-12. Antimicrob Agents Chemother 15, 182-9.
Seley, K. L., Salim, S. & Zhang, L. (2005). “Molecular chameleons”. Design and synthesis of C-4-substituted imidazole fleximers. Org Lett 7, 63-6.
Shah, K., Liu, Y., Deirmengian, C. & Shokat, K. M. (1997). Engineering unnatural nucleotide specificity for Rous sarcoma virus tyrosine kinase to uniquely label its direct substrates. Proc Natl Acad Sci U S A 94, 3565-70.
Shimba, N., Nomura, A. M., Marneft, A. B. & Craik, C. S. (2004). Herpesvirus protease inhibition by dimer disruption. J Virol 78, 6657-65.
Sia, S. K. & Kim, P. S. (2003). Protein grafting of an HIV-1-inhibiting epitope. Proc Natl Acad Sci U S A 100, 9756-61.
Singleton, S. F., Shan, F., Kanan, M. W., McIntosh, C. M., Stearman, C. J., Helm, J. S. & Webb, K. J. (2001). Facile synthesis of a fluorescent deoxycytidine analogue suitable for probing the RecA nucleoprotein filament. Org Lett 3, 3919-22.
Singleton, S. F., Simonette, R. A., Sharma, N. C. & Roca, A. I. (2002). Intein-mediated affinity-fusion purification of the Escherichia coli RecA protein. Protein Expr Purif 26, 476-88.
Singleton, S. F., Simonette, R. A., Sharma, N. C. & Roca, A. I. (2002). Intein-mediated affinity-fusion purification of the Escherichia coli RecA protein. Protein Expr Purif 26, 476-88
Spraft, B. G. (1994). Resistance to antibiotics mediated by target alterations. Science 264, 388-93.
Stohl, E. A., Blount, L. & Seifert, H. S. (2002). Differential cross-contemplation patterns of Escherichia coli and Neisseria gonorrhoeae RecA proteins. Microbiology 148, 1821-31.
Story, R. M., Weber, I. T. & Steitz, T. A. (1992). The structure of the E. coli recA protein monomer and polymer. Nature 355, 318-25.
U.S. Pat. No. 5,628,984.
U.S. Pat. No. 6,180,082.
Vaara, M. (1992). Agents that increase the permeability of the outer membrane. Microbiol Rev 56, 395-411.
Vasilieva, S. (2002). SOS Chromotest methodology for fundamental genetic research. Res Microbiol 153, 435-40.
Wang, W., Purwanto, M. G. & Weisz, K. (2004). CG base pair recognition by substituted phenylimidazole nucleosides. Org Biomol Chem 2, 1194-8.
Weber, T. P., Widger, W. R. & Kohn, H. (2003). Metal-1,4-dithio-2,3-dihydroxybutane chelates: novel inhibitors of the Rho transcription termination factor. Biochemistry 42, 9121-6.
Weigle, J. J. (1953). Induction of mutation in a bacterial virus. Proc Natl Acad Sci U S A 39, 628-636.
West, S. C., Cassuto, E., Mursalim, J. & Howard-Flanders, P. (1980). Recognition of duplex DNA containing single-stranded regions by RecA protein. Proceedings of the National Academy of Sciences of the United States of America 77, 2569-2573.
Yarranton, G. T. & Sedgwick, S. G. (1982). Cloned truncated recA genes in E. coli II. Effects of truncated gene products on in vivo recA+protein activity. Mol. Gen. Genet. 185, 209-217.
Yu, X., Horiguchi, T., Shigesada, K. & Egelman, E. H. (2000). Three-dimensional reconstruction of transcription termination factor rho: orientation of the N-terminal domain and visualization of an RNA-binding site. J Mol Biol 299, 1279-87.
Zaitsev, E. N. & Kowalczykowski, S. C. (1998). Essential monomer-monomer contacts define the minimal length for the N-terminus of RecA protein. Mol Microbiol 29, 1317-8.
Zondlo, N. J. & Schepartz, A. (1999). Highly specific DNA recognition by a designed, miniature protein. J Am Chem Soc 121, 6938-6939.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A compound for modulating RecA protein activity, wherein the compound modulates RecA activity by interfering with assembly of monomeric RecA protein subunits into a nucleoprotein filament.

2. The compound of claim 1, wherein the compound inhibits RecA activity.

3. The compound of claim 1, wherein the compound is a mimetic of the N-terminal helical domain of the RecA protein.

4. The compound of claim 3, wherein the N-terminal helical domain of the RecA protein comprises amino acid residues 1-31 of E. coli RecA protein.

5. The compound of claim 3, wherein the compound comprises the amino acid sequence B—X₃-Z-X₂-Z-Z-X₂-Z-X₃-Z, wherein B is lysine or arginine;
Xₙ is n number of any amino acids and X can be the same or different amino acids; and Z is alanine, valine, leucine, isoleucine, phenylalanine, or methionine.

6. The compound of claim 3, wherein the compound is selected as a mimetic of the N-terminal helical domain of the RecA protein utilizing an mRNA display assay.

7. A compound for modulating RecA protein activity, wherein the compound modulates RecA activity by interfering with adenosine triphosphate hydrolysis by the RecA protein.

8. The compound of claim 7, wherein the compound inhibits RecA activity.

9. The compound of claim 7, wherein the compound is selected from the group consisting of a modified adenosine, a modified 5-propynyl-deoxyuridine, a curcumin derivative and a bismuth-dithiol complex.

10. The compound of claim 9, wherein the compound is a modified adenosine selected from the group consisting of a modified adenosine monophosphate, a modified adenosine diphosphate and a modified adenosine triphosphate.

11. The compound of claim 9, wherein the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate.

12. The compound of claim 9, wherein the compound has the general formula (I): 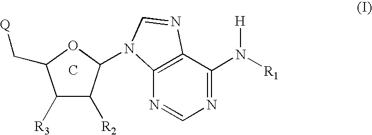
wherein:
R₁ is selected from the group consisting of: 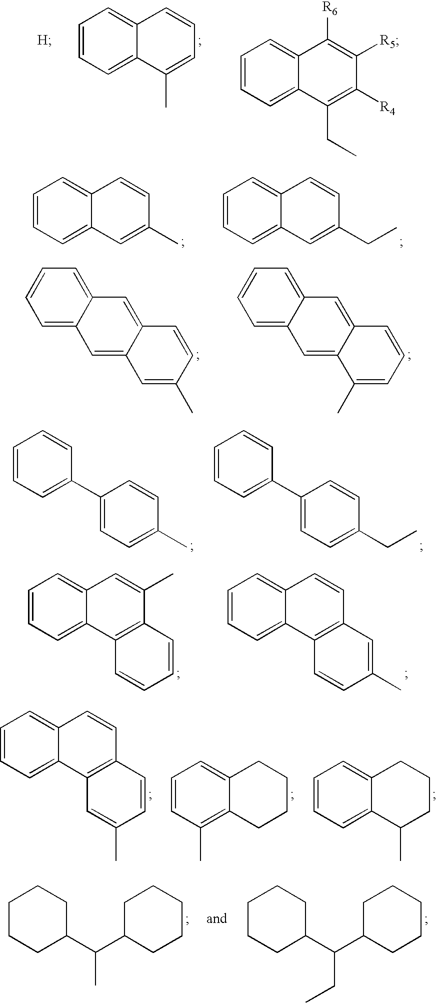
wherein R₄, R₅ and R₆ are each independently selected from the group consisting of H, alkyl, substituted alkyl, OH, alkoxyl, and substituted alkoxyl;
R₂ and R₃ are each independently selected from the group consisting of H, F, OH, NH₂ and Y-Z-R₇,
Y is selected from the group consisting of O and NR₂₂, and wherein R₂₂ is selected from the group consisting of H, alkyl, substituted alkyl, and alkoxyl;
Z is selected from the group consisting of (CH₂)ₚ, CF₂ and C═O, and wherein p is an integer from 1 to 8; and
R₇ is selected from the group consisting of H, alkyl, substituted alkyl, alkenyl, substituted alkenyl, alkynyl, substituted alkynyl, aryl and substituted aryl, or
R₂ and R₃ can together with ring C form the following five-membered heterocyclic ring structure: 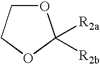
wherein R₂ₐ and R_{2b} are each independently selected from the group consisting of H, alkyl, substituted alkyl, alkenyl, substituted alkenyl, alkynyl, substituted alkynyl, aryl and substituted aryl; and
Q is selected from the group consisting of: 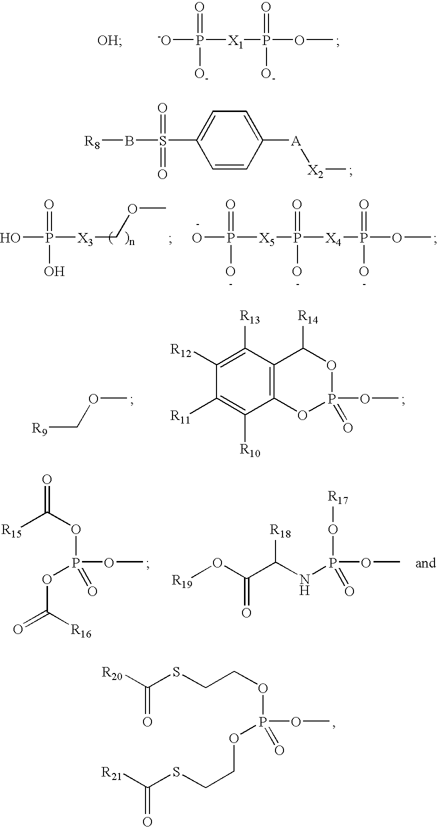
wherein X₁, X₂, X₃, X₄ and X₅ are each independently selected from the group consisting of O, NR₂₃, CH₂ and CF₂, and wherein R₂₃ is selected from the group consisting of H, alkyl, substituted alkyl, and alkoxyl;
A and B are each independently selected from the group consisting of O, NR₂₄, CH₂, CF₂ and C═O, and wherein R₂₄ is selected from the group consisting of H, alkyl, substituted alkyl, and alkoxyl;
n is an integer from 0 to 4;
R₈ is selected from the group consisting of H, alkyl, substituted alkyl, alkenyl, substituted alkenyl, alkynyl, substituted alkynyl, aryl and substituted aryl;
R₉ is selected from the group consisting of 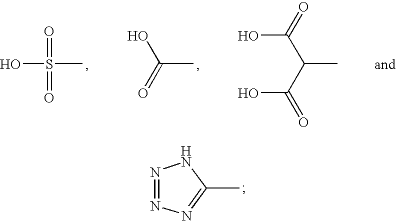
and
R₁₀, R₁₁, R₁₂, R₁₃, R₁₄, R₁₅, R₁₆, R₁₇, R₁₈, R₁₉, R₂₀ and R₂₁, are each independently selected from the group consisting of H, alkyl, substituted alkyl, alkenyl, substituted alkenyl, alkynyl, substituted alkynyl, aryl and substituted aryl;
or a pharmaceutically acceptable salt thereof;
subject to the proviso that the compound of formula (I) is not 2′,3′-O-(N-methyl-anthraniloyl)-adenosine-5′diphosphate, 2′, (3′)-O-(2,4,6-trinitrophenyl)-adenosine-5′-diphosphate, N⁶-(1-napthyl)-adenosine-5′-O-Diphosphate, N⁶-(1-benzyl)-adenosine-5′-O-Diphosphate, or N⁶-(2-phenethyl)-adenosine-5′-O-Diphosphate.

13. The compound of claim 9, wherein the compound has the general formula (II): 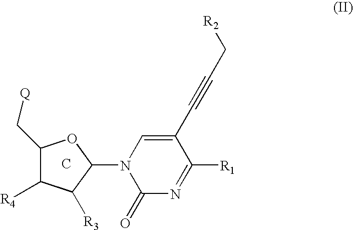
wherein:
R₁ and R₂ are each independently selected from the group consisting of H, alkyl, substituted alkyl, F, Cl, Br, OH, NR₂₂R₂₃ and Y-Z-R₂₄, wherein
R₂₂ and R₂₃ are each independently selected from the group consisting of H, alkyl, substituted alkyl, and alkoxyl;
Y is selected from the group consisting of O, S, NR₂₅ and (CH₂)ₚ, and wherein R₂₅ is selected from the group consisting of H, alkyl, substituted alkyl, and alkoxyl and p is an integer from 1 to 8;
Z can be present or absent and when present is selected from the group consisting of O, S, NR₂₆R₂₇ and C═O, and wherein R₂₆ and R₂₇ are each independently selected from the group consisting of H, alkyl, substituted alkyl, and alkoxyl; and
R₂₄ is selected from the group consisting of H, alkyl, substituted alkyl, alkenyl, substituted alkenyl, alkynyl, substituted alkynyl, aryl and substituted aryl;
R₃ and R₄ are each independently selected from the group consisting of H, F, OH, NR₂₈R₂₉ and R₅—R₆—R₇, wherein R₂₈ and R₂₉ are each independently selected from the group consisting of H, alkyl, substituted alkyl, and alkoxyl;
R₅ is selected from the group consisting of O and NR₃₀, and wherein R₃₀ is selected from the group consisting of H, alkyl, substituted alkyl, and alkoxyl;
R₆ is selected from the group consisting of (CH₂)_{q}, CF₂ and C═O, and wherein q is an integer from 1 to 8; and
R₇ can be present or absent and when present is selected from the group consisting of H, alkyl, substituted alkyl, alkenyl, substituted alkenyl, alkynyl, substituted alkynyl, aryl and substituted aryl, or
R₃ and R₄ can together with ring C form the following five-membered heterocyclic ring structure: 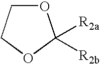
wherein R₂ₐ and R_{2b} are each independently selected from the group consisting of H, alkyl, substituted alkyl, alkenyl, substituted alkenyl, alkynyl, substituted alkynyl, aryl and substituted aryl; and
Q is selected from the group consisting of: 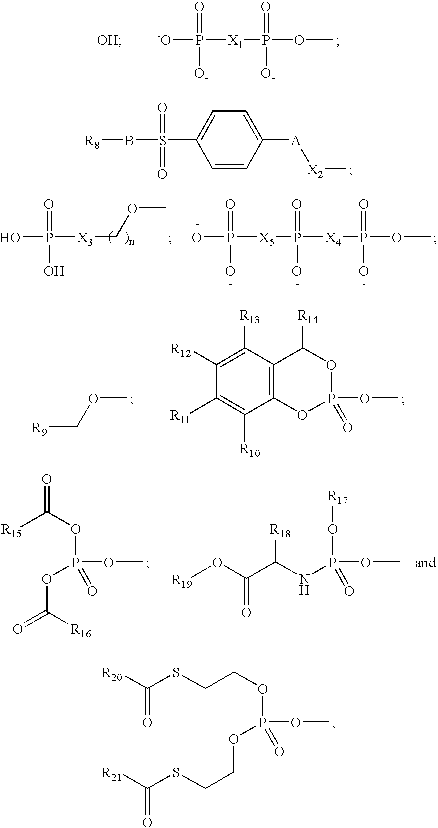
wherein X₁, X₂, X₃, X₄ and X₅ are each independently selected from the group consisting of O, NR₃₁, CH₂ and CF₂, and wherein R₃₁ is selected from the group consisting of H, alkyl, substituted alkyl, and alkoxyl; A and B are each independently selected from the group consisting of O, NR₃₂, CH₂, CF₂ and C═O, and wherein R₃₂ is selected from the group consisting of H, alkyl, substituted alkyl, and alkoxyl;
n is an integer from 0 to 4;
R₈ is selected from the group consisting of H, alkyl, substituted alkyl, alkenyl, substituted alkenyl, alkynyl, substituted alkynyl, aryl and substituted aryl;
R₉ is selected from the group consisting of 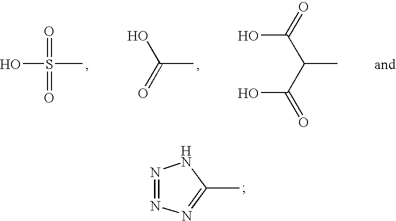
and
R₁₀, R₁₁, R₁₂, R₁₃, R₁₄, R₁₅, R₁₆, R₁₇, R₁₈, R₁₉, R₂₀ and R₂₁ are each independently selected from the group consisting of H, alkyl, substituted alkyl, alkenyl, substituted alkenyl, alkynyl, substituted alkynyl, aryl and substituted aryl;
or a pharmaceutically acceptable salt thereof.

14. The compound of claim 9, wherein the compound has the general formula (III): 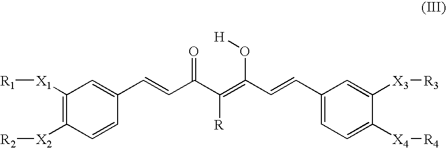
wherein:
X₁, X₂, X₃ and X₄ are each independently selected from the group consisting of H, F, (CH₂)ₙ, CF₂, C═O, O and NR₅, and wherein n is an integer from 1 to 8 and R₅ is selected from the group consisting of H, alkyl, substituted alkyl, and alkoxyl; and
R, R₁, R₂, R₃ and R₄ can each independently be present or absent and if present each is independently selected from the group consisting of H, alkyl, substituted alkyl, alkenyl, substituted alkenyl, alkynyl, substituted alkynyl, aryl and substituted aryl;
or a pharmaceutically acceptable salt thereof.

15. The compound of claim 9, wherein the compound has the general formula (IV): 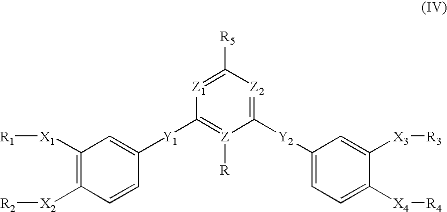
wherein:
X₁, X₂, X₃ and X₄ are each independently selected from the group consisting of H, F, (CH₂)ₙ, CF₂, C═O, O and NR₆, and wherein n is an integer from 1 to 8 and R₆ is selected from the group consisting of H, alkyl, substituted alkyl, and alkoxyl;
Y₁ and Y₂ are each independently selected from the group consisting of C≡C and HC═CH;
Z₁ is selected from the group consisting of CH and N;
Z₂ is selected from the group consisting of CH and N; and
R, R₁, R₂, R₃ R₄ and R₅ can each independently be present or absent and if present each is independently selected from the group consisting of H, alkyl, substituted alkyl, alkenyl, substituted alkenyl, alkynyl, substituted alkynyl, aryl and substituted aryl;
or a pharmaceutically acceptable salt thereof.

16. The compound of claim 9, wherein the bismuth-dithiol complex is comprised of a bismuth(III) ion and a dithiol compound, wherein the dithiol compound has the general formula (V): 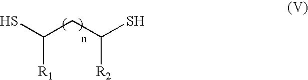
wherein:
n is an integer from 0 to 2; and
R₁ and R₂ are each independently selected from the group consisting of H, alkyl, substituted alkyl, alkenyl, substituted alkenyl, alkynyl, substituted alkynyl, aryl and substituted aryl, or
R₁ and R₂ can together form a five-, six- or seven-membered cycloalkyl, substituted cycloalkyl, heterocyclic, substituted heterocyclic, aryl, or substituted aryl ring structure;
or a pharmaceutically acceptable salt thereof;
subject to the proviso that the compound of formula (V) is not 2,3-dimercapto-1-propanol.

17. A method for identifying compounds that modulate RecA protein activity, the method comprising:
a) contacting a candidate compound with a RecA protein; and
b) determining whether the candidate compound modulates the activity of the RecA protein.

18. The method of claim 17, wherein determining whether the candidate compound modulates the activity of the RecA protein comprises measuring the interference of assembly of monomeric RecA protein subunits into a nucleoprotein filament when the candidate compound contacts the RecA protein.

19. The method of claim 18, wherein measuring the interference of assembly of monomeric RecA protein subunits into the nucleoprotein filament comprises measuring the amount of monomeric RecA protein subunits released from the nucleoprotein filament.

20. The method of claim 17, wherein determining whether the candidate compound modulates the activity of the RecA protein comprises measuring inhibition of adenosine triphosphate (ATP) hydrolysis by the RecA protein.

21. The method of claim 20, wherein measuring inhibition of ATP hydrolysis by the RecA protein comprises measuring the decrease in production of phosphate resulting from inhibition of the RecA protein ATP hydrolysis by the candidate compound.

22. The method of claim 20, wherein measuring inhibition of ATP hydrolysis by the RecA protein comprises measuring the decrease in the production of ADP resulting from inhibition of the RecA protein ATP hydrolysis by the candidate compound.

23. The method of claim 17, wherein determining whether the candidate compound modulates the activity of the RecA protein comprises measuring the interference of assembly of monomeric RecA protein subunits into a nucleoprotein filament when the candidate compound contacts the RecA protein and measuring inhibition of adenosine triphosphate (ATP) hydrolysis by the RecA protein.

24. A method of inhibiting RecA protein activity in a bacterium, comprising contacting the RecA protein with a compound that interferes with assembly of monomeric RecA protein subunits into a nucleoprotein filament.

25. The method of claim 24, wherein the compound is a mimetic of the N-terminal helical domain of the RecA protein.

26. The method of claim 25, wherein the N-terminal helical domain of the RecA protein comprises amino acid residues 1-31 of E. coli RecA protein.

27. The method of claim 25, wherein the compound comprises the amino acid sequence B—X₃-Z-X₂-Z-Z-X₂-Z-X₃-Z, wherein B is lysine or arginine;
Xₙ is n number of any amino acids and X can be the same or different amino acids; and Z is alanine, valine, leucine, isoleucine, phenylalanine, or methionine.

28. A method of inhibiting RecA protein activity in a bacterium, comprising contacting the RecA protein with a compound that interferes with adenosine triphosphate hydrolysis by the RecA protein.

29. The method of claim 28, wherein the compound is selected from the group consisting of a modified adenosine, a modified 5-propynyl-deoxyuridine, a curcumin derivative and a bismuth-dithiol complex.

30. The method of claim 29, wherein the compound is a modified adenosine selected from the group consisting of a modified adenosine monophosphate, a modified adenosine diphosphate and a modified adenosine triphosphate.

31. The method of claim 29, wherein the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate.

32. The method of claim 29, wherein the compound has the general formula (I): 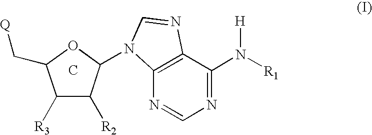
wherein:
R₁ is selected from the group consisting of: 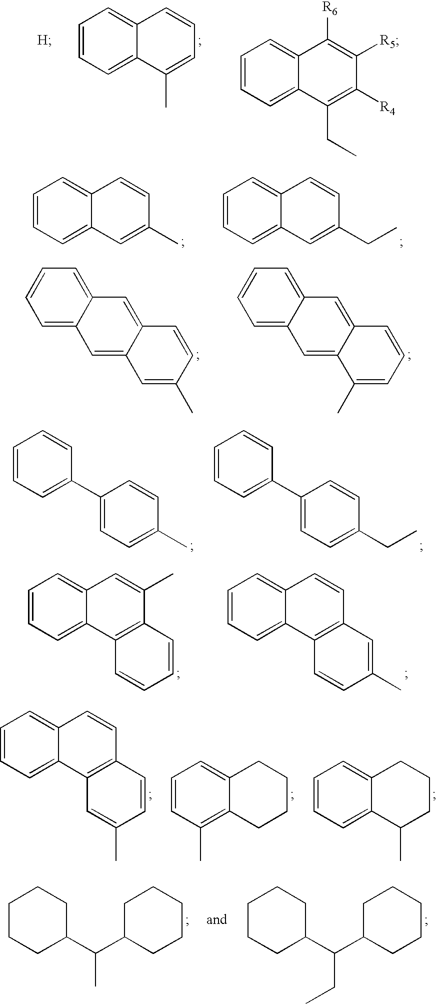
wherein R₄, R₅ and R₆ are each independently selected from the group consisting of H, alkyl, substituted alkyl, OH, alkoxyl, and substituted alkoxyl;
R₂ and R₃ are each independently selected from the group consisting of H, F, OH, NH₂ and Y-Z-R₇,
wherein Y is selected from the group consisting of O and NR₂₂, and wherein R₂₂ is selected from the group consisting of H, alkyl, substituted alkyl, and alkoxyl;
Z is selected from the group consisting of (CH₂)ₚ, CF₂ and C═O, and wherein p is an integer from 1 to 8; and
R₇ is selected from the group consisting of H, alkyl, substituted alkyl, alkenyl, substituted alkenyl, alkynyl, substituted alkynyl, aryl and substituted aryl, or
R₂ and R₃ can together with ring C form the following five-membered heterocyclic ring structure: 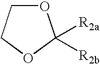
wherein R₂ₐ and R_{2b} are each independently selected from the group consisting of H, alkyl, substituted alkyl, alkenyl, substituted alkenyl, alkynyl, substituted alkynyl, aryl and substituted aryl; and
Q is selected from the group consisting of: 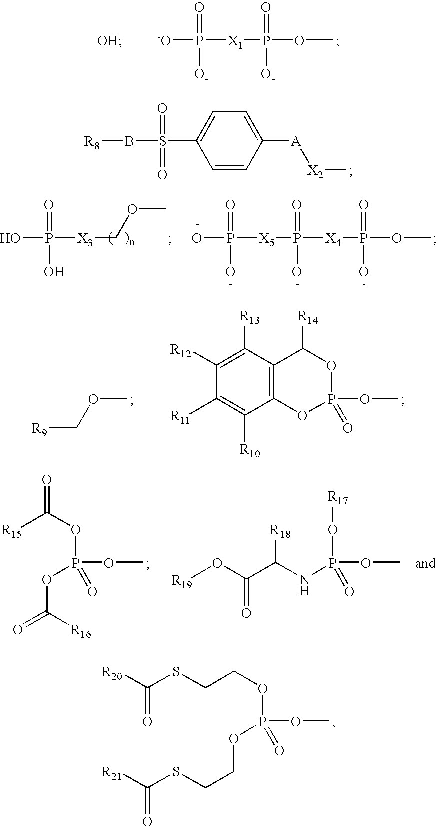
wherein X₁, X₂, X₃, X₄ and X₅ are each independently selected from the group consisting of O, NR₂₃, CH₂ and CF₂, and wherein R₂₃ is selected from the group consisting of H, alkyl, substituted alkyl, and alkoxyl;
A and B are each independently selected from the group consisting of O, NR₂₄, CH₂, CF₂ and C═O, and wherein R₂₄ is selected from the group consisting of H, alkyl, substituted alkyl, and alkoxyl;
n is an integer from 0 to 4;
R₈ is selected from the group consisting of H, alkyl, substituted alkyl, alkenyl, substituted alkenyl, alkynyl, substituted alkynyl, aryl and substituted aryl;
R₉ is selected from the group consisting of 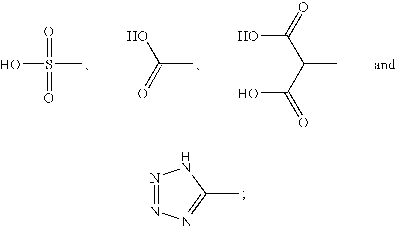
and
R₁₀, R₁₁, R₁₂, R₁₃, R₁₄, R₁₅, R₁₆, R₁₇, R₁₈, R₁₉, R₂₀ and R₂₁, are each independently selected from the group consisting of H, alkyl, substituted alkyl, alkenyl, substituted alkenyl, alkynyl, substituted alkynyl, aryl and substituted aryl;
or a pharmaceutically acceptable salt thereof.

33. The method of claim 29, wherein the compound has the general formula (II): 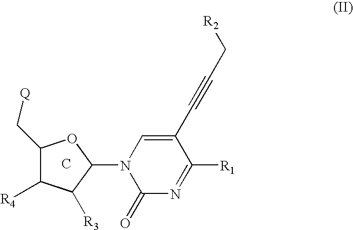
wherein:
R₁ and R₂ are each independently selected from the group consisting of H, alkyl, substituted alkyl, F, Cl, Br, OH, NR₂₂R₂₃ and Y-Z-R₂₄, wherein
R₂₂ and R₂₃ are each independently selected from the group consisting of H, alkyl, substituted alkyl, and alkoxyl;
Y is selected from the group consisting of O, S, NR₂₅ and (CH₂)ₚ, and wherein R₂₅ is selected from the group consisting of H, alkyl, substituted alkyl, and alkoxyl and p is an integer from 1 to 8;
Z can be present or absent and when present is selected from the group consisting of O, S, NR₂₆R₂₇ and C═O, and wherein R₂₆ and R₂₇ are each independently selected from the group consisting of H, alkyl, substituted alkyl, and alkoxyl; and
R₂₄ is selected from the group consisting of H, alkyl, substituted alkyl, alkenyl, substituted alkenyl, alkynyl, substituted alkynyl, aryl and substituted aryl;
R₃ and R₄ are each independently selected from the group consisting of H, F, OH, NR₂₈R₂₉ and R₅—R₆—R₇,
wherein R₂₈ and R₂₉ are each independently selected from the group consisting of H, alkyl, substituted alkyl, and alkoxyl;
R₅ is selected from the group consisting of O and NR₃₀, and wherein R₃₀ is selected from the group consisting of H, alkyl, substituted alkyl, and alkoxyl;
R₆ is selected from the group consisting of (CH₂)_{q}, CF₂ and C═O, and
wherein q is an integer from 1 to 8; and
R₇ can be present or absent and when present is selected from the group consisting of H, alkyl, substituted alkyl, alkenyl, substituted alkenyl, alkynyl, substituted alkynyl, aryl and substituted aryl, or
R₃ and R₄ can together with ring C form the following five-membered heterocyclic ring structure: 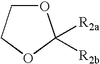
wherein R₂ₐ and R_{2b} are each independently selected from the group consisting of H, alkyl, substituted alkyl, alkenyl, substituted alkenyl, alkynyl, substituted alkynyl, aryl and substituted aryl; and
Q is selected from the group consisting of: 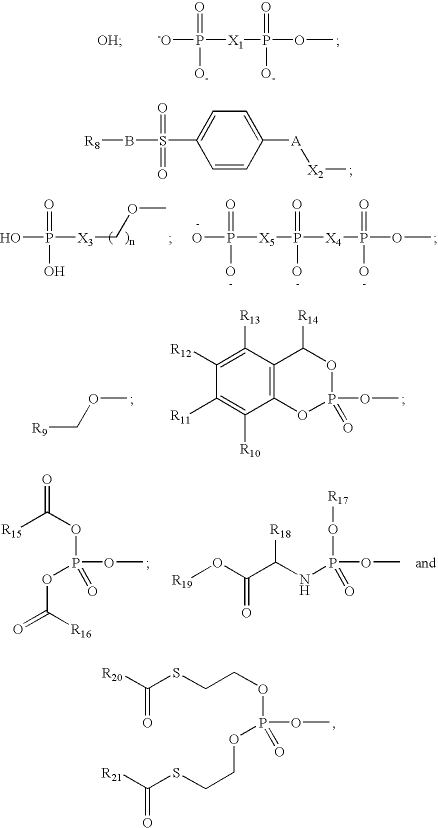
wherein X₁, X₂, X₃, X₄ and X₅ are each independently selected from the group consisting of O, NR₃₁, CH₂ and CF₂, and wherein R₃₁ is selected from the group consisting of H, alkyl, substituted alkyl, and alkoxyl;
A and B are each independently selected from the group consisting of O, NR₃₂, CH₂, CF₂ and C═O, and wherein R₃₂ is selected from the group consisting of H, alkyl, substituted alkyl, and alkoxyl;
n is an integer from 0 to 4;
R₈ is selected from the group consisting of H, alkyl, substituted alkyl, alkenyl, substituted alkenyl, alkynyl, substituted alkynyl, aryl and substituted aryl;
R₉ is selected from the group consisting of 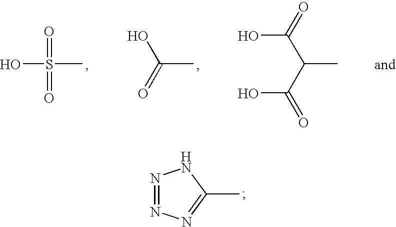
and
R₁₀, R₁₁, R₁₂, R₁₃, R₁₄, R₁₅, R₁₆, R₁₇, R₁₈, R₁₉, R₂₀ and R₂₁ are each independently selected from the group consisting of H, alkyl, substituted alkyl, alkenyl, substituted alkenyl, alkynyl, substituted alkynyl, aryl and substituted aryl;
or a pharmaceutically acceptable salt thereof.

34. The method of claim 29, wherein the compound has the general formula (III): 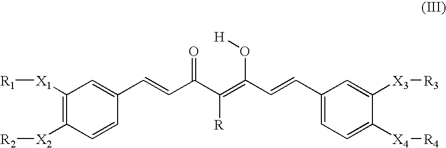
wherein:
X₁, X₂, X₃ and X₄ are each independently selected from the group consisting of H, F, (CH₂)ₙ, CF₂, C═O, O and NR₅, and wherein n is an integer from 1 to 8 and R₅ is selected from the group consisting of H, alkyl, substituted alkyl, and alkoxyl; and
R, R₁, R₂, R₃ and R₄ can each independently be present or absent and if present each is independently selected from the group consisting of H, alkyl, substituted alkyl, alkenyl, substituted alkenyl, alkynyl, substituted alkynyl, aryl and substituted aryl;
or a pharmaceutically acceptable salt thereof.

35. The method of claim 29, wherein the compound has the general formula (IV): 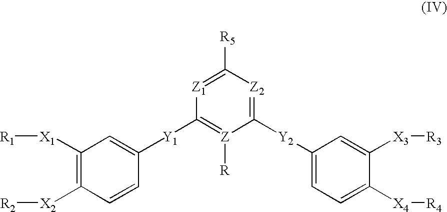
wherein:
X₁, X₂, X₃ and X₄ are each independently selected from the group consisting of H, F, (CH₂)ₙ, CF₂, C═O, O and NR₆, and wherein n is an integer from 1 to 8 and R₆ is selected from the group consisting of H, alkyl, substituted alkyl, and alkoxyl;
Y₁ and Y₂ are each independently selected from the group consisting of C≡C and HC═CH;
Z₁ is selected from the group consisting of CH and N;
Z₂ is selected from the group consisting of CH and N; and
R, R₁, R₂, R₃ R₄ and R₅ can each independently be present or absent and if present each is independently selected from the group consisting of H, alkyl, substituted alkyl, alkenyl, substituted alkenyl, alkynyl, substituted alkynyl, aryl and substituted aryl;
or a pharmaceutically acceptable salt thereof.

36. The method of claim 29, wherein the bismuth-dithiol complex is comprised of a bismuth(III) ion and a dithiol compound, wherein the dithiol compound has the general formula (V): 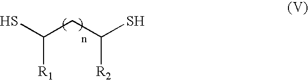
wherein:
n is an integer from 0 to 2; and
R₁ and R₂ are each independently selected from the group consisting of H, alkyl, substituted alkyl, alkenyl, substituted alkenyl, alkynyl, substituted alkynyl, aryl and substituted aryl, or
R₁ and R₂ can together form a five-, six- or seven-membered cycloalkyl, substituted cycloalkyl, heterocyclic, substituted heterocyclic, aryl, or substituted aryl ring structure;
or a pharmaceutically acceptable salt thereof.

37. A method of treating a bacterial infection in a subject, comprising administering to the subject a therapeutically effective amount of a pharmaceutical composition comprising a compound that modulates bacterial RecA protein activity.

38. The method of claim 37, wherein the compound modulates RecA activity by interfering with assembly of monomeric RecA protein subunits into a nucleoprotein filament.

39. The method of claim 38, wherein the compound is a mimetic of the N-terminal helical domain of the RecA protein.

40. The method of claim 39, wherein the N-terminal helical domain of the RecA protein comprises amino acid residues 1-31 of E. coli RecA protein.

41. The method of claim 39, wherein the compound comprises the amino acid sequence B—X₃-Z-X₂-Z-Z-X₂-Z-X₃-Z, wherein B is lysine or arginine;
Xₙ is n number of any amino acids and X can be the same or different amino acids; and Z is alanine, valine, leucine, isoleucine, phenylalanine, or methionine.

42. The method of claim 37, wherein the compound modulates RecA activity by interfering with adenosine triphosphate hydrolysis by the RecA protein.

43. The method of claim 42, wherein the compound is selected from the group consisting of a modified adenosine, a modified 5-propynyl-eoxyuridine, a curcumin derivative and a bismuth-dithiol complex.

44. The method of claim 43, wherein the compound is a modified adenosine selected from the group consisting of a modified adenosine monophosphate, a modified adenosine diphosphate and a modified adenosine triphosphate.

45. The method of claim 43, wherein the compound is a modified 5-propynyl-deoxyuridine selected from the group consisting of a modified 5-propynyl-deoxyuridine monophosphate, a modified 5-propynyl-deoxyuridine diphosphate and a modified 5-propynyl-deoxyuridine triphosphate.

46. The method of claim 43, wherein the compound has the general formula (I): 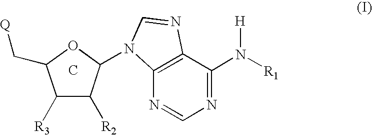
wherein:
R₁ is selected from the group consisting of: 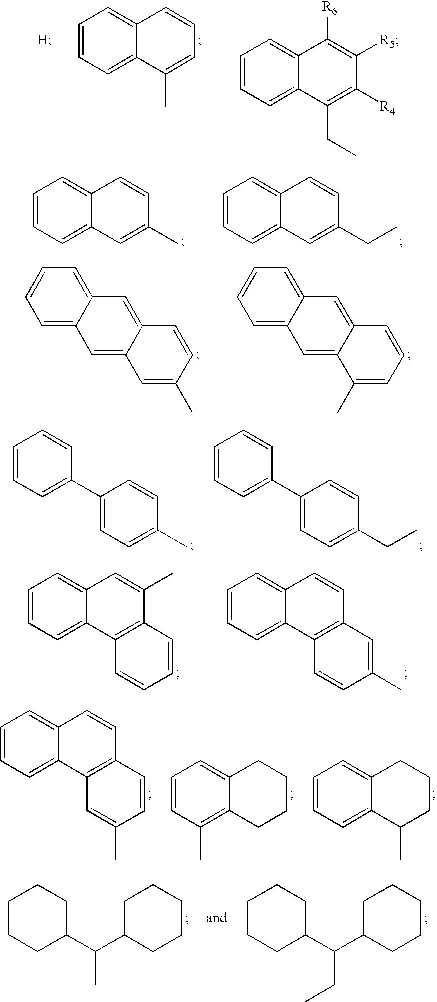
wherein R₄, R₅ and R₆ are each independently selected from the group consisting of H, alkyl, substituted alkyl, OH, alkoxyl, and substituted alkoxyl;
R₂ and R₃ are each independently selected from the group consisting of H, F, OH, NH₂ and Y-Z-R₇,
wherein Y is selected from the group consisting of O and NR₂₂, and wherein R₂₂ is selected from the group consisting of H, alkyl, substituted alkyl, and alkoxyl;
Z is selected from the group consisting of (CH₂)ₚ, CF₂ and C═O, and wherein p is an integer from 1 to 8; and
R₇ is selected from the group consisting of H, alkyl, substituted alkyl, alkenyl, substituted alkenyl, alkynyl, substituted alkynyl, aryl and substituted aryl, or
R₂ and R₃ can together with ring C form the following five-membered heterocyclic ring structure: 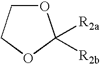
wherein R₂ₐ and R_{2b} are each independently selected from the group consisting of H, alkyl, substituted alkyl, alkenyl, substituted alkenyl, alkynyl, substituted alkynyl, aryl and substituted aryl; and
Q is selected from the group consisting of: 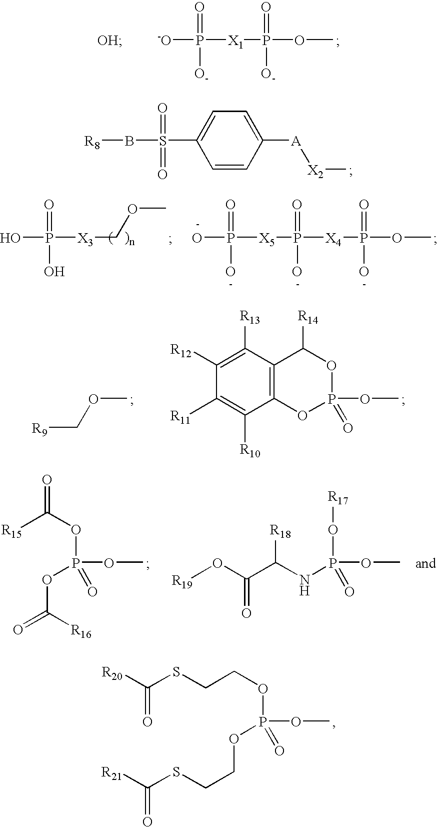
wherein X₁, X₂, X₃, X₄ and X₅ are each independently selected from the group consisting of O, NR₂₃, CH₂ and CF₂, and wherein R₂₃ is selected from the group consisting of H, alkyl, substituted alkyl, and alkoxyl;
A and B are each independently selected from the group consisting of O, NR₂₄, CH₂, CF₂ and C═O, and wherein R₂₄ is selected from the group consisting of H, alkyl, substituted alkyl, and alkoxyl;
n is an integer from 0 to 4;
R₈ is selected from the group consisting of H, alkyl, substituted alkyl, alkenyl, substituted alkenyl, alkynyl, substituted alkynyl, aryl and substituted aryl;
R₉ is selected from the group consisting of 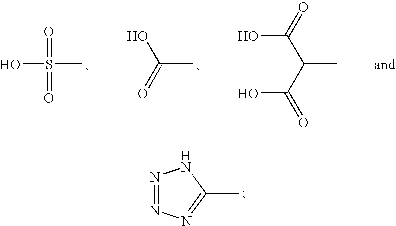
and
R₁₀, R₁₁, R₁₂, R₁₃, R₁₄, R₁₅, R₁₆, R₁₇, R₁₈, R₁₉, R₂₀ and R₂₁, are each independently selected from the group consisting of H, alkyl, substituted alkyl, alkenyl, substituted alkenyl, alkynyl, substituted alkynyl, aryl and substituted aryl;
or a pharmaceutically acceptable salt thereof.

47. The method of claim 43, wherein the compound has the general formula (II): 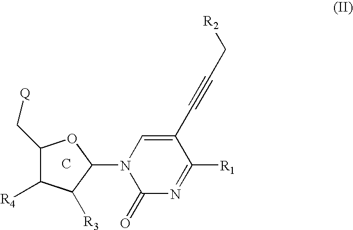
wherein:
R₁ and R₂ are each independently selected from the group consisting of H, alkyl, substituted alkyl, F, Cl, Br, OH, NR₂₂R₂₃ and Y-Z-R₂₄, wherein
R₂₂ and R₂₃ are each independently selected from the group consisting of H, alkyl, substituted alkyl, and alkoxyl;
Y is selected from the group consisting of O, S, NR₂₅ and (CH₂)ₚ, and wherein R₂₅ is selected from the group consisting of H, alkyl, substituted alkyl, and alkoxyl and p is an integer from 1 to 8;
Z can be present or absent and when present is selected from the group consisting of O, S, NR₂₆R₂₇ and C═O, and wherein R₂₆ and R₂₇ are each independently selected from the group consisting of H, alkyl, substituted alkyl, and alkoxyl; and
R₂₄ is selected from the group consisting of H, alkyl, substituted alkyl, alkenyl, substituted alkenyl, alkynyl, substituted alkynyl, aryl and substituted aryl;
R₃ and R₄ are each independently selected from the group consisting of H, F, OH, NR₂₈R₂₉ and R₅—R₆—R₇, wherein R₂₈ and R₂₉ are each independently selected from the group consisting of H, alkyl, substituted alkyl, and alkoxyl;
R₅ is selected from the group consisting of O and NR₃₀, and wherein R₃₀ is selected from the group consisting of H, alkyl, substituted alkyl, and alkoxyl;
R₆ is selected from the group consisting of (CH₂)_{q}, CF₂ and C═O, and
wherein q is an integer from 1 to 8; and
R₇ can be present or absent and when present is selected from the group consisting of H, alkyl, substituted alkyl, alkenyl, substituted alkenyl, alkynyl, substituted alkynyl, aryl and substituted aryl, or
R₃ and R₄ can together with ring C form the following five-membered heterocyclic ring structure: 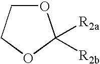
wherein R₂ₐ and R_{2b} are each independently selected from the group consisting of H, alkyl, substituted alkyl, alkenyl, substituted alkenyl, alkynyl, substituted alkynyl, aryl and substituted aryl; and
Q is selected from the group consisting of: 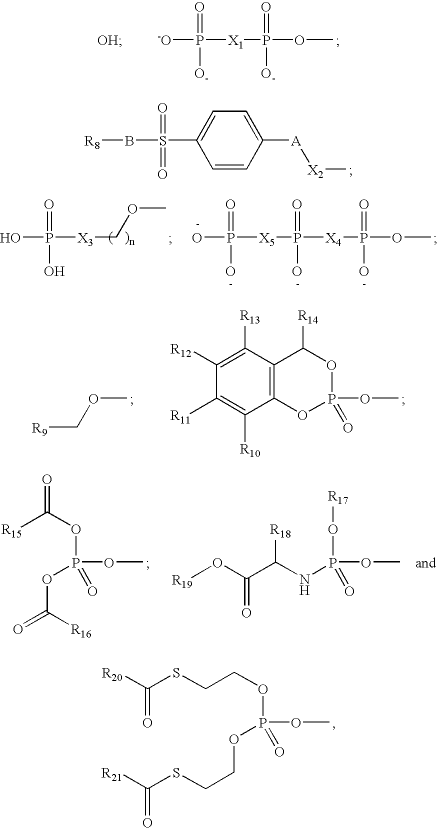
wherein X₁, X₂, X₃, X₄ and X₅ are each independently selected from the group consisting of O, NR₃₁, CH₂ and CF₂, and wherein R₃₁ is selected from the group consisting of H, alkyl, substituted alkyl, and alkoxyl;
A and B are each independently selected from the group consisting of O, NR₃₂, CH₂, CF₂ and C═O, and wherein R₃₂ is selected from the group consisting of H, alkyl, substituted alkyl, and alkoxyl;
n is an integer from 0 to 4;
R₈ is selected from the group consisting of H, alkyl, substituted alkyl, alkenyl, substituted alkenyl, alkynyl, substituted alkynyl, aryl and substituted aryl;
R₉ is selected from the group consisting of 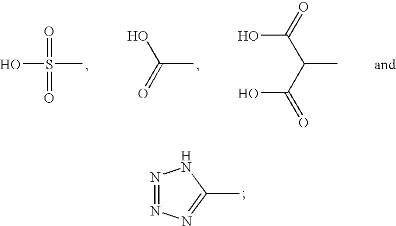
and
R₁₀, R₁₁, R₁₂, R₁₃, R₁₄, R₁₅, R₁₆, R₁₇, R₁₈, R₁₉, R₂₀ and R₂₁ are each independently selected from the group consisting of H, alkyl, substituted alkyl, alkenyl, substituted alkenyl, alkynyl, substituted alkynyl, aryl and substituted aryl;
or a pharmaceutically acceptable salt thereof.

48. The method of claim 43, wherein the compound has the general formula (III): 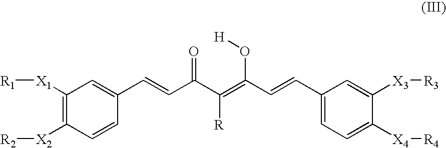
wherein:
X₁, X₂, X₃ and X₄ are each independently selected from the group consisting of H, F, (CH₂)ₙ, CF₂, C═O, O and NR₅, and wherein n is an integer from 1 to 8 and R₅ is selected from the group consisting of H, alkyl, substituted alkyl, and alkoxyl; and
R, R₁, R₂, R₃ and R₄ can each independently be present or absent and if present each is independently selected from the group consisting of H, alkyl, substituted alkyl, alkenyl, substituted alkenyl, alkynyl, substituted alkynyl, aryl and substituted aryl;
or a pharmaceutically acceptable salt thereof.

49. The method of claim 43, wherein the compound has the general formula (IV): 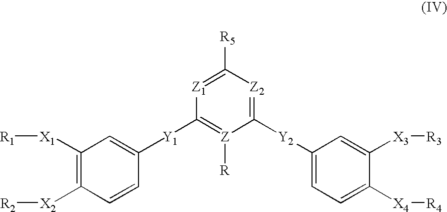
wherein:
X₁, X₂, X₃ and X₄ are each independently selected from the group consisting of H, F, (CH₂)ₙ, CF₂, C═O, O and NR₆, and wherein n is an integer from 1 to 8 and R₆ is selected from the group consisting of H, alkyl, substituted alkyl, and alkoxyl;
Y₁ and Y₂ are each independently selected from the group consisting of C≡C and HC═CH;
Z₁ is selected from the group consisting of CH and N;
Z₂ is selected from the group consisting of CH and N; and
R, R₁, R₂, R₃ R₄ and R₅ can each independently be present or absent and if present each is independently selected from the group consisting of H, alkyl, substituted alkyl, alkenyl, substituted alkenyl, alkynyl, substituted alkynyl, aryl and substituted aryl; or a pharmaceutically acceptable salt thereof.

50. The method of claim 43, wherein the bismuth-dithiol complex is comprised of a bismuth(III) ion and a dithiol compound, wherein the dithiol compound has the general formula (V): 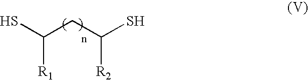
wherein:
n is an integer from 0 to 2; and
R₁ and R₂ are each independently selected from the group consisting of H, alkyl, substituted alkyl, alkenyl, substituted alkenyl, alkynyl, substituted alkynyl, aryl and substituted aryl, or
R₁ and R₂ can together form a five-, six- or seven-membered cycloalkyl, substituted cycloalkyl, heterocyclic, substituted heterocyclic, aryl, or substituted aryl ring structure;
or a pharmaceutically acceptable salt thereof.

51. The method of claim 37, wherein the pharmaceutical composition further comprises an antibiotic.

52. The method of claim 51, wherein the antibiotic is a replication inhibitor.

53. The method of claim 52, wherein the replication inhibitor is selected from the group consisting of actinomycins, adriamycin, aflatoxins, altromycins, anthramycin, bleomycins, calicheamicins, carmustine (BCNU), daunomycin, distamycins, dynemicins, echinomycin, esperamicins, kericidin, mitomycins, neocarzinostatin, netropsins, nitric oxide, nitrogen mustards, nitrosamines, peroxides, pluramycins, pyrrolo[1,4]benzodiazepines, sibiromycin, streptozotocin, tomamycin, beta-lactams, quinolones, fluoroquinolones, DNA Gyrase inhibitors, DNA Polymerase I inhibitors, nucleoside and nucleotide analogs, ribonucleotide reductase inhibitors, antifolates, and DNA biosynthesis inhibitors.

54. A composition comprising a compound of claim 1 and a pharmaceutically acceptable carrier.
